SEQUENCE LISTING (21 sequences)

SEQ ID NO: 1 - DNA, 1196 bp, synthetic construct
  source 1..1196: mol_type = other DNA
tcttatgaagttagcggcggacgggtgagtaacacgtgggtaacctgcccataagactgggataactccgggaaaccggggctaataccggataacattttgaaccgcatggttcgaaattgaaaggcggcttcggctgtcacttatggatggacccgcgtcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgaaaaccctagagatagggcttctccttcgggagcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaagagctgcaagaccgcgaggtg

SEQ ID NO: 2 - DNA, 1134 bp, synthetic construct
  source 1..1134: mol_type = other DNA
cgagcgaacagaaaaggaagntgctcctttgacgttagcggcggacgggtgagtaacacgtgggcaacctacccctantagtttgggataactccgggaanccggggctaataccgaataatctcttttgcttcatggtnaaagactgaaagacggtttcggctgtcgctataggatgggcccgcggcgcattagctagttggtgaggtaacggctcaccaaggcgacgatgcgtagccgacctgagagggtgatcggccacactgggactgagacaccggcccagactcctacgggaggcagcagtagggaatcttccacaatgggcgaaagcctgatggagcaacgccgcgtgagtgaagaaggttttcggatcgtaaaactctgttgtaagggaagaacaagtacagtagtaactggctgtaccttgacggtaccttattagaaagccacggctaactacgtgccagcagccgcggtaatacgtaggtggcaagcgttgtccggaattattgggcgtaaagcgcgcgcaggcggtcctttaagtctgatgtgaaagcccacggctcaaccgtggagggtcattggaaactgggggacttgagtgcagaagaggaaagtggaattccaagtgtagcggtgaaatgcgtagagatttggaggaacaccagtggcgaaggcgactttctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcccgttgaccactgtagagatatagtttccccttcgggggcaacggtgacaggtggnggcatggttgtcgtcagctcgtgtcgtgagatgntggggttaagtcccgcaacgagcgcaacccttgatcttagttgccatcatttagttgggcactctaaggtgactgccggtgacaaaccggaggagg

SEQ ID NO: 3 - DNA, 1230 bp, synthetic construct
  source 1..1230: mol_type = other DNA
aatacatgcaagtcgagcgaatggattaagagcttgctcttatgaagttagcggcggacgggtgagtaacacgtgggtaacctgcccataagactgggataactccgggaaaccggggctaataccggataacattttgaaccgcatggttcgaaattgaaaggcggcttcggctgtcacttatggatggacccgcgtcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaaccctagagatagggcttctccttcgggagcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaagagctgcaagaccgcgag

SEQ ID NO: 4 - DNA, 1197 bp, synthetic construct
  source 1..1197: mol_type = other DNA
ggatggttgtttgaaccgcatggttcagacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttggggttaagtcccgcaacgagcgcaacccttgatcttagttgccagcattcagttgggcactctaaggtgactgccggtgacaaaccggaggaagggtgggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatca

SEQ ID NO: 5 - DNA, 1253 bp, synthetic construct
  source 1..1253: mol_type = other DNA
cgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttggggttaagtcccgcaacgagcgcaacccttgatcttagttgccagcattcagttgggcactctaaggtgactgccggtgacaaaccggaggaagggtggggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatca

SEQ ID NO: 6 - DNA, 1131 bp, synthetic construct
  source 1..1131: mol_type = other DNA
taacctacctataagactgggataacttcgggaaaccggagntaataccggataacatgttgaaccgcatggttcaacagtgaaagacggtcttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgaagaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtgtgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcnaatcatcntgccncttatgattngggctacacacgtgctacaatggacaata

SEQ ID NO: 7 - DNA, 1200 bp, synthetic construct
  source 1..1200: mol_type = other DNA
ttctatcaccaaaaacaggtcaattaacagtaggggatacacaagacttccagttggtagaaaacctgtcggatgggagcaatcaagaccaaacaggaaatacaacatttactgttagcgattcatccattgccacaatacaaaacaacaaactaacagcggttgctccagggaccgtgacggtaacggcaacctatggtagctcatccgacacagcaacaattacggtgaaaaagcctacaccagttccgccatcggtaccacagcctgtaaatccagtggaaccaaagccttcaggtgcaatagacattattcgtactgttgaacaaggtatcgtgaagtatcgagcagacgtatcattgaatcatgtccaaatgctagtacaacaaatgactaatcaagatgcacgaacaattcgtctcgtgtacccagcagaaacagcaactgcaaaggcttatttgaatctttttagaaacgcaggattgtttttatacaatcaaaatacggatcttttcatacaaacagagttagcacaaatgaggattccttttaaatcattcgatggcgtgacagaggatgtgtacttccatctagctcctgtcaagggaccacaacaggatatgattcatacgaatgccctaaacaatgaacaaatccagaaagcaatgccagatagagcgataatttcattactaggtacacccgtaagaatcgaaacgaatctacaaaatcgcccagtaatgattacgttaccgatttcgtctgagttaacagaagatcaaattgcctcactacttatctatgttgagcatagtgacgaaacaacggaagtaaaacatgggcgtatagtggagtttgtacctggggtgaaaggtttccagtttgaagttgatcacttctcaacatatagcttagtatatacttcagaagttcaagaagctgaggaagaagtagagcaaatagcaccatatattcaaggtttcccagatggcacatttaaaccaaatgcttcggttacacgtgcacaaatggcaacaatgttagcacgttttttaacgaatggtgacataccaacggcaagtgcaacttttaaagatacaaagaatcatccttctaaagatgcgattgaattagtgaaagaaattggtttatttaacggtataacggatacaacatttaatccaaatgggacgattacaa

SEQ ID NO: 8 - DNA, 1200 bp, synthetic construct
  source 1..1200: mol_type = other DNA
cgctactggcgttaccggggctactggtgttactggcgctaccggcgctaccggcgctaccggcgttactggcgttactggttctacgggtgtaaccggcgctactggttctaccggcgctaccggcgttactggagtaactggggctactggtgttgctggttctacgggcgctactggcgcaatcggacctactggcgctactggcgttactggcgctaccggggctactggggtaactggggctaccggcgttactggggtaaccggcgctactggcgctactggtgtaaccggggctaccggggctactggcttgactggcgcaatcggacctactggcaccactgggataactggggctaccggcgttactggcgttactggcgctaccggcgttgctggggctacgggtgagactggcgctactggggtaactggggctaccggtgttgctggggctacgggcgctacgggcgttactggcgcaatcggacctactggcgctactggtgtaaccggggctaccggcgttactggcgttactggggctaccggcgttactggggtaaccggcgctactggcttgactggtgcaatcggacctactggcgctactggagtaaccggggctaccggcgttactggggctactggcgttactggggtaactggggctaccggcgttactggcgttactggcgctaccggcgttgctggcgttaccggcgttactggcgctactggcgttactggttctacgggagtgaccggcgctactggtattactggttctacgggtgtaaccggggctactggcgctactggttctactggggtaaccggcgttactggttctacgggtgagactggggtaactggggctaccggcattactggagtaaccggcgctactggcattactggagtaaccggcgctactggcactactggagtaactggcgctaccggcgttactggcgctaccggcgttgctggggctacgggtgagaccggcgctactggcactactggagtaactggggctaccggtgttgctggttctacgggcgcgactggcgcaatcggacctactggcgttactggtgtaactggggctaccggcgcaatcggacctactggcgttactggagctaccggcgttgctggggtaactggggctactggcgttactggtgc

SEQ ID NO: 9 - DNA, 1200 bp, synthetic construct
  source 1..1200: mol_type = other DNA
tataaatccagtaatgcttttttcttttgttcgtttacattcataaaatcgtgtaaccttttaagctttttccctttctgctcttctacattgctttgaagttttaatgtaacatactgcccttttttcacgagtaaatcaataataaatctacgaaaatcatgatattgaataggtacatgatttacacaaccatactttcctgctcttctatactgtgagcactttaaatatttccattcagtccgttcaccattcttctttcgaagataggattgtacgataaccatatttgaaccacaaatcgcgcattttgctaaattacgaaattcattccaagctgtgatttttgttttactcataataatgtctttttgatttgctcgattccatatctcttcatctacaattttgggatggtgattaggaaaaacaaaccacttttctctcggattgcgaatttgttttttcctaccgttcaccttgaccgaggtatattgatttaaaataaacgtccctttgtaaattggattttgaagaatacgctgaacggatgttatttgccaattttttttagttttagattttatccccattttatttaactcatttgaaatttttttatatccccacccattgatataccaactatatatccttcgaacaacattagattcctcttcattaataatcaacttttgattaatacggtcatacccatacggtactttaccaatatgctcacctcttcttactttcaccgccaaagcagcagtaatactcgaagataatgtccgactatactgcgccgaaaataaagaccataactcaaatgccatatcatttttcccagcttttacagaatcatagccttcttccacagaaacaattctaacattatgagctaaaagcacttctcttatctctaatgagtcctttaaatcacgcgctaaacgagaaatagacttaaagacaaccatatcaatttctttcgccttcgccttttgtaataataattgaatagcaggacgatccataaataaagtaccactcatcccctcatctttaaaaacacatttttcattccacataaatccattacgctccaaccaattcctacaaatatctatttggttttcaatcgaggaaacttgttcatcacgatctgttgatacgcgaacataaacagcataagattcc

SEQ ID NO: 10 - DNA, 1200 bp, synthetic construct
  source 1..1200: mol_type = other DNA
ctgttttagtcctcctaagtaatatttcataacttcatattttgcatgtgtatcatcagatagtagaatgtcaatttcatattctaaaatcggcaacttatgatatatgaagtcgaaaaaatctgtttcggttgtgaattcaaattgttttaaagtatctttgaaaaatctttctcgattgagtaataattgcaacaaatgattttctaaatgctttatttctcgcatttttttatgtatggctttttgtttagctatttctttttgcatgtttttattatttagatttacatctaaattcaattcttttaaaagaaaagattctgcttgtttgtgcgtccatccatgcaatatcatcagacctgaaaattggtctattggtcttctattcccgcaccctgcgaaacaataacagttgttgtctgatacgttcacaaccatgctatctttgttatctgcatggaagggacacttgcacatgtaattttttccccgctttctaacagttaccccgcaatagctttctaagaaatttataataggaagtttacttttaattagatcgcttttttgcatatcatcacacttctttatctttgaaaataattttgatatgtttagcttggttttttcctcgttgatagaaaacatctgtaagacctttttcctccaaacctttgaaggctcgtccaacgctagagcgtgataaattgcaatgttctattacatcttcaatcttaattttttcgtgttgttctgcacaaccttgctgatgtagatagagcaatacaagtttttcagtaggtgttgtttcttggacttcgaaaatgtcttttatcgtcaatattctgctcctcctgtcaaatttctttatgaatttaactttaacagatattttcttatgcgtcaatatgacacataagaattatataacgaaaataattaaatcctttaatataaacgatttctagcgagtaataaaaacgattccttgattcattttcggaaacacctgacgtcttccctacgattccttatattttttgcggggattattcatgtttctttgtcaatcagcaaggacaaacaatcctcgctattttgagagtgtattttcatattccttcatgattttgtagaatgatgtttttttgagatttagcatttgactgaattctacacctttgatttctttgcttt

SEQ ID NO: 11 - DNA, 1200 bp, synthetic construct
  source 1..1200: mol_type = other DNA
gtttctcccgttgctccggttactcctgttgccccagttgggccggttgctccgattgaacctgttgctccggttggaccggtctctcccgttgggccgatttctccagttacaccggttgcgccggtttctcccgttgaaccaattgagccggttactcctgttgcccctgttgctccagttacaccagtcaagccggttgggcctgttattcctgttattcctgttgctccggttgaaccggatgcacccgtcgccccggtttctcctgtcgatcccgttggaccagtctccccagttgacccggttgatcctgtcgcgcctgttactcccgttgggcctgttgggcctgttgagccggtcgttcccgttgctcctgtcggcccggttattcctgtcgcaccggtttctccggttactcccgtcgccccggttactccagttgaacccgtttctcctgtcgatcctgttgatccggttacaccagttgagcccgtgaccccagtcgggccagtcactcctgttgatcctgttgcaccagttactccggttgggcctgttgaacctgtcgatcccgttgcgccggttgggcctgctgcgccagttgagccggttgagccagttggcccgatttctccagttgaaccggtcgctcctgtctccccggtcgctcctgttgggccgatcgggccagtagcccctgtggctcccgttatccccgttgccccagtcactccagttggacccgtttctcccgttgagccagttgctcctgtttctcctgttggaccagtctccccagttgacccggttgatccggtttctcctgtcgatcccgttgcgccggttattcctgttacaccggtctctcctattgcgccggttgggcctgttactccagtcgctccggttggaccggtttctccggttgaaccagttgaccctgctgctcctgttggtccagttactcccgttgaaccagttgatcccgttataccagttaccccagtcgctcctgtcgatcccattgcacccgttgagccggtggctcctgttactcccgttgctccggttgagccggttgggccggttgggccggttacacccgtgaccccagtcgggccagtaactcctgttgcccctgttactccggtcgatcccgtttctcctgttgcacctgttactccggtctctcctgtt

SEQ ID NO: 12 - DNA, 1200 bp, synthetic construct
  source 1..1200: mol_type = other DNA
aaaactgtcagtgtaatggatacacctgatttcccggagatttttattccaacagctaaaggccaaggtcgtaacttgcaagaactaaaggaaacttctgatattaactggacatttatcagtccttcagcggtatttgacccagacgggaaaagaactggattttatcagtcaggaaaagatcatcttcttgtgaattcgaaaggcgaaagttatatcagctatgcagactatgcaattgcagtattggatgaaattgaaaatccaaaacatataaatgaacgctttacagttgttggagaagctgaataagtgattgagtagaaacaaagggtaccattagtgtcatcgctaatggtatcttccttctaagaatgtacggagacattgaaaactttgcaatcagagtagctgattattaaaaaggggatttatcacgttccaatgaatattatcgagctgatgacagagtaggtgcttagtgagttggaccaaaaaaatatgtatgtgtataaaaagctctgactaagaccttttggattagttagagcttctttatttgactgtgttacccagtgtaatcctccgtattgtttaatacactttagtttgaaaatccgggaaactcttctacagttcctatttcgtgtttaaagtgtgaatattccagcggggaactttactgaaatgaggggtattttcgaaaacacaccagtatgctctatgacctgccgtcaatgacattaaagaaatgatttgatgtaaaaaggagcgatattcgttaaaatttaggtagtagccatacatagccatataaaacacatgattaattctaaattctttactgatcgttcttaaatttgatatacttgaaatacgttagatctaactgttttgctactcaaatgagaatgatcgttctttaattatttaagaatgagggagggatgaatgcatcaatgattctcaggcctttctcagaataaagaggtaatacattctaaggtgggattacatggctagaaataaagaatttgatgaaaaaaaagcattaagaaaagcaatggatcttttctgggaacagggttatgaaaaaacatccatgcaagacttggtggaccatatgggcattcaccgcagaagtatttatgatacatttggcgacaaacatactttatttatgcgagccttaagtcagta

SEQ ID NO: 13 - DNA, 1200 bp, synthetic construct; identical to SEQ ID NO: 11
  source 1..1200: mol_type = other DNA

SEQ ID NO: 14 - DNA, 1200 bp, synthetic construct
  source 1..1200: mol_type = other DNA
aatataaacaaattgtgctaaatccgctttatagtataagtaacaaaacgatagataaacaaacaacacaagttcattcatcgttttaaattaattaactcacgcattatcacattttactgaaggagtgtttgtaatggaaaaattattcgacgcaattagaaacacagtcgatgctggaatcaaccaagattggacaaaattaggaactagcattgttgacatcgttgacaatggtgtaaaagttatttctaaatttattggtgcataattcagattattaatatcgttttaataataaaggagagattataatgcaaaaattagcagaagcaatcgcaaacacagtaaaagcaggacaagaccatgattggacaaaattaggtacaagcatcgttgatatcgtagaaaacggtgtaagtgcattaactaaagtattcggtggttaattttcgataaataagaacttaattataaataaaataaactaaaggagagactataatgactaaattagcagaagcaatcgcaaacgcagtaaaagcaggacaagaccaagattgggcaaaattaggtacaagcatcgtaggtatcgcagaaaacggaatcggtttattaggtaaagtattcggattctaatatatgattagatggaccagggcaagcgctctggtctttttttatttgcagattatttgttattattaaaaagaataagaagtttgttcattaaaatttgaaacggggataaattgtgacatataaacatatcttattagatttcgatgatacaatagttgatttttacgatgcagaggaaaaggcattttataatatggcaaaacattacggtcattttccgactaaacaagatttccaacattttagaaaggtcaatcaagcacactgggaagcttttcaacaaaatgaattgacgaaagaacaagttttatctcatcgatttattaattatttcaatgattattacatagaagtagatggtaaagaagcggatgagatatttagagatgaattagctaaagcgccgcttaaatttttcgatcaaacgattgaaactattaaccagctgaaagataaacattcattatatattgttacgaatggtgtgacaatcacgcaacagcgtcgtattgctcagacaaattttaatgatatatttaatggaatatttatttcaga

SEQ ID NO: 15 - DNA, 1200 bp, synthetic construct
  source 1..1200: mol_type = other DNA
cactcgtgccaagagctaatgatagcccaagccctgtaacaattacttcaactaatgatggagttgtcgtaatttgcgtaattgcttgaataacgataggtgctccgagtgagattaaactcgatccagtacctggtacaggtactccattcacttggattgtaagacctcctaatagacttgcagttgcagtgttagcgataacagtgattttatagaatccagtttcactaattacgaaagtatcagcatctaattgagaaattgctgtaccaaactgagatccaacagtattaaatggtactggatcattaattcctaaatctaaagaaatcccaccggagttaaatgcgtatagtcctgctggaagtcctagtcctgatggtccagtcggtccagtcggtccagtagccccagtcggtccagtagctccagtagctccagtgtcaccagtcggccccgtaggtccagtagctccagtgtcaccagtcggccccgtaggtccagtagccccagtgtcaccagtcggccccgtaggtccagtagccccagtgtcaccagtcggccccgtaggtccagtagtcccagtgtcaccagtcggtccagtcggccccgtaggtccagtcggcccagtcggccccgtaggtccagtcggtccagttggcccggtaggtacagtcggccccgtaggtccagtcggcccagtcggcccggtcggcccagtcggaagggtaaacggtggtatcggtggtaatgtaggtcctacaagattagggtcaaatgcactagctgataaagattcatcgggatttaatccatttgaataattattatttgacataaattcacctccataaagcgttcattatatagtagatgcaaaaccgaaagaaaatgacacggacatttgaattattgaaaagaaatcttaaactacttgaacaatttaaaaaaatggaaagtttagtatatgtataacatatgattgatttggaagagggtgattatgttgaacaagcaaggaattacaattagtttatgtatgattgttcgagatgaggaggagacaatagcccgttgtttagacacagttgaaaaaattgtggatgaaattatagtggttgatacaggctccgtcgatcgaacgaaagaaatcgtagagaaatacacttctaacatatatgatttccagtggattga

SEQ ID NO: 16 - DNA, 1212 bp, synthetic construct
  source 1..1212: mol_type = other DNA
aatacatgcaagtcgagcgaacagataaggagcttgctcctttgacgttagcggcggacgggtgagtaacacgtgggtaacctacntataagactggaataactccgggaaaccggggctaatgccggataacatttagaaccgcatggttctaaagtgaaagatggttttgctatcacttatagatggacccgcgccgtattagctagttggtaaggtaacggcttaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggtttcttaagtctgatgtgaaagcccacggctcnnccgtggagggtcattggaaactgggaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaaacttagttgccagcatttagttgggcactctaagttgactgccggtgacaaaccggaggaaggtggggatgacgtcaaatcatcatgccccttatgatttgggctacacacgtgctacaatggacaatacaaa

SEQ ID NO: 17 - DNA, 1116 bp, synthetic construct
  source 1..1116: mol_type = other DNA
cgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgaagaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgatccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggtagcaaaaccgcgaggtcaagcaaatcccataaagttgttctcagttcggattgtagtctgcaactcgactacatgaagctggaatcgctagtaatcg

SEQ ID NO: 18 - DNA, 670 bp, synthetic construct
  source 1..670: mol_type = other DNA
ctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattc

SEQ ID NO: 19 - DNA, 1068 bp, synthetic construct
  source 1..1068: mol_type = other DNA
catggttcgaaattgaaaggcggcttcggctgtcacttatggatggacccgcgtcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgaaaaccctagagatagggcttctccttcgggagcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaa

SEQ ID NO: 20 - DNA, 1144 bp, synthetic construct
  source 1..1144: mol_type = other DNA
aaccggggctaataccgaataacacttttgacctcatggtcgaatgttaaaagacggtttcggctgtcactacaggatgggcccgcggcgcattagctagttggtgaggtaacggctcaccaaggcaacgatgcgtagccgacctgagagggtgatcggccacactgggactgagacacggcccagactcctacgggaggcagcagtagggaatcttccacaatggacgaaagtctgatggagcaacgccgcgtgagtgaagaaggatttcggttcgtaaaactctgttgcaagggaagaacaagtagcgtagtaactggcgctaccttgacggtaccttgttagaaagccacggctaactacgtgccagcagccgcggtaatacgtaggtggcaagcgttgtccggaattattgggcgtaaagcgcgcgcaggtggtttcttaagtctgatgtgaaagcccacggctcaaccgtggagggtcattggaaactgggaaacttgagtgcagaagaggatagtggaattccaagtgtagcggtgaaatgcgtagagatttggaggaacaccagtggcgaaggcgactatctggtctgtaactgacactgaggcgcgaaagcgtggggagcaaacaggattagataccctggtagtccacgccgtaaacgatgagtgctaagtgttggggggtttccgcccctcagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcccattgaccactgtagagatacagttttcccttcggggacaacggtgacaggtggtgcatggttgtcgtcagctcgtgtcgtgagatgttgggttaagtcccgcaacgagcgcaacccttattcttagttgccatcatttagttgggcactctaaggagactgccggtgacaaaccggaggaaggtggggatgacgtcaaatcatcatgccccttatgacctgggctacacacgtgctacaatggacggtacaaacggttgccaacccgcgagggggagctaatccgataaaaccg

SEQ ID NO: 21 - DNA, 977 bp, synthetic construct
  source 1..977: mol_type = other DNA
tgtggatgggctcacggcctatcagcttgttggtggggtaatggcctaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagcacggaagaagcgaaagtgacggtacgtgcagaagaagcgccggctaactacgtgccagcagccgcggtaatacgtagggcgcaagcgttgtccggaattattgggcgtaaagagctcgtaggcggtttgtcgcgtctgctgtgaaagcccggggcttaaccccgggtgtgcagtgggtacgggcagacttgagtgcagtaggggagactggaactcctggtgtagcggtgaaatgcgcagatatcaggaagaacaccgatggcgaaggcaggtctctgggctgttactgacgctgaggagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatacaccggatcggctcagagatgagttttcctccttgtggggctggtgtacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggatgacgtcaaatcatcatgccccttat

(19) United States
(12) Patent Application Publication
(10) Pub. No.: US 2025/0268955 A1
(43) Pub. Date: Aug. 28, 2025

(54) MICROBIAL COMPOSITIONS FOR THE TREATMENT OF SKIN DISEASES

(71) Applicant: Concerto Biosciences, Inc., Cambridge, MA (US)

(72) Inventors: Jared KEHE, Cambridge, MA (US); Bernardo CERVANTES, Cambridge, MA (US); Cheri ACKERMAN, Somerville, MA (US); Abdulrahman HASSABALLAH, Boston, MA (US); Keaton ARMENTROUT, Seattle, WA (US)

(21) Appl. No.: 19/205,443

(22) Filed: May 12, 2025

Related U.S. Application Data
(63) Continuation of application No. 18/825,396, filed on Sep. 5, 2024, now Pat. No. 12,329,787.
Application No. 18/825,396 is a continuation of application No. 18/589,539, filed on Feb. 28, 2024, now Pat. No. 12,115,197.
Application No. 18/589,539 is a continuation of application No. PCT/US2022/079637, filed on Nov. 10, 2022.
(60) Provisional application No. 63/278,134, filed on Nov. 11, 2021.

(51) Int. Cl. A61K 35/741; A61K 31/51; A61P 31/04; C12N 1/20; C12R 1/07

(52) U.S. Cl. CPC A61K 35/741; A61K 31/51; A61P 31/04; C12N 1/20; C12R 2001/07

(57) ABSTRACT

Provided herein are compositions, methods, kits, and devices for the treatment of skin diseases. Also provided herein are isolated and purified bacteria, excipients, carriers, dosage forms and routes of administration for such bacteria. Furthermore, provided herein are isolated and purified mixtures of bacteria, excipients, carriers, dosage forms and routes of administration for such mixtures. Also provided herein are conditions for treatment with bacteria and bacterial mixtures.

### CROSS-REFERENCE

This application is a continuation of U.S. application Ser. No. 18/825,396, filed Sep. 5, 2024, which is a continuation of U.S. application Ser. No. 18/589,539, filed Feb. 28, 2024, now U.S. Pat. No. 12,115,197, issued Oct. 15, 2024, which is a continuation of International Application No. PCT/US2022/079637, filed Nov. 10, 2022, which claims the benefit of U.S. Provisional Application No. 63/278,134, filed Nov. 11, 2021, the disclosures of which are incorporated herein by reference in their entirety.

### SEQUENCE LISTING

The application contains a Sequence Listing which has been submitted electronically in XML format and is hereby incorporated by reference in its entirety. Said XML copy was created on Jan. 16, 2024, is named as 202421-701301_SL and is 43,274 bytes in size.

### SUMMARY OF THE INVENTION

Disclosed herein are compositions. In some embodiments, a composition comprises: bacterial strains that are purified, wherein the bacterial strains comprise: a first strain, which comprises a 16s rRNA sequence with at least 97% sequence identity to SEQ ID NO: 4; a second strain, which comprises a 16s rRNA sequence with at least 97% sequence identity to SEQ ID NO: 1; and a third strain, which comprises a 16s rRNA sequence with at least 97% sequence identity to SEQ ID NO: 2; and wherein (a) the bacterial strains are lyophilized; or (b) the composition is formulated for delivery to a skin. In some embodiments, the first strain comprises the 16s rRNA sequence with at least 97% sequence identity over at least 1000 bases to SEQ ID NO: 4. In some embodiments, the second strain comprises the 16s rRNA sequence with at least 97% sequence identity over at least 1000 bases to SEQ ID NO: 1. In some embodiments, the third strain comprises the 16s rRNA sequence with at least 97% sequence identity over at least 1000 bases to SEQ ID NO: 2. In some embodiments, the first strain further comprises a sequence with at least 95% sequence identity to SEQ ID NO: 11, SEQ ID NO: 12, or both. In some embodiments, the first strain further comprises having a sequence of SEQ ID NO: 11, SEQ ID NO: 12, or both. In some embodiments, the second strain further comprises a sequence with at least 95% sequence identity to SEQ ID NO: 15. In some embodiments, the second strain further comprises having a sequence of SEQ ID NO: 15. In some embodiments, the third strain further comprises sequence with at least 95% sequence identity to SEQ ID NO: 7, SEQ ID NO: 8, or both. In some embodiments, the third strain further comprises having a sequence of SEQ ID NO: 7, SEQ ID NO: 8, or both. In some embodiments, the composition is formulated as: a suspension, an emulsion, a cream, a lotion, a tincture, a gel, a foam, a powder, an ointment, a paste, or an oil. In some embodiments, the composition further comprises a fourth bacterial strain. In some embodiments, the composition comprises at least 10\hat{ }3 colony forming units (cfu) per gram of bacteria. In some embodiments, the composition comprises 10\hat{ }3 to 10\hat{ }12 colony forming units (cfu) per gram of bacteria. In some embodiments, the bacterial strains are grown in aerobic conditions. In some embodiments, the bacterial strains are grown without animal products. In some embodiments, the bacterial strains are grown in Tryptic Soy Broth (TSB). In some embodiments, the composition when stored in a sealed container placed at 20° C. retains at least about: 10\hat{ }4 cfu after 6 months, as measured by cfu counts. In some embodiments, the composition further comprises an excipient. In some embodiments, the composition further comprises a lyoprotectant. In some embodiments, the composition further comprises an emollient. In some embodiments, the composition further comprises or a salt thereof. In some embodiments, the bacterial strains when contacted with S. aureus cause a reduction in expression of at least one of the following genes: gmk, agr, psma, saeR, ccpA, and SigB in S. aureus, wherein the reduction in expression is measured by a fluorescence reporter assay. In some embodiments, the bacterial strains are present in an amount effective to suppress virulence of S. aureus as measured by a reduction in expression of at least one of the following genes: gmk, agr, psma, saeR, ccpA, and SigB in S. aureus wherein the reduction in expression is measured by a fluorescence reporter assay.

In some embodiments, a composition comprises: bacterial strains that are purified, wherein the bacterial strains comprise: a first strain, which comprises a 16s rRNA sequence with at least 97% sequence identity to SEQ ID NO: 5; a second strain, which comprises a 16s rRNA sequence with at least 97% sequence identity to SEQ ID NO: 1; and a third strain, which comprises a 16s rRNA sequence with at least 97% sequence identity to SEQ ID NO: 2; and wherein (a) the bacterial strains are lyophilized; or (b) the composition is formulated for delivery to a skin. In some embodiments, the first strain comprises the 16s rRNA sequence with at least 97% sequence identity over at least 1000 bases to SEQ ID NO: 5. In some embodiments, the second strain comprises the 16s rRNA sequence with at least 97% sequence identity over at least 1000 bases to SEQ ID NO: 1. In some embodiments, the third strain comprises the 16s rRNA sequence with at least 97% sequence identity over at least 1000 bases to SEQ ID NO: 2. In some embodiments, the first strain further comprises a sequence with at least 95% sequence identity to SEQ ID NO: 13. In some embodiments, the first strain further comprises having a sequence of SEQ ID NO: 13. In some embodiments, the second strain further comprises a sequence with at least 95% sequence identity to SEQ ID NO: 15. In some embodiments, the second strain further comprises having a sequence of SEQ ID NO: 15. In some embodiments, the third strain further comprises a sequence with at least 95% sequence identity to SEQ ID NO: 7, SEQ ID NO: 8, or both. In some embodiments, the third strain further comprises having a sequence of SEQ ID NO: 7, SEQ ID NO: 8, or both. In some embodiments, the composition is formulated as: a suspension, an emulsion, a cream, a lotion, a tincture, a gel, a foam, a powder, an ointment, a paste, or an oil. In some embodiments, the composition further comprises a fourth bacterial strain. In some embodiments, the composition comprises at least 10\hat{ }3 colony forming units (cfu) per gram of bacteria. In some embodiments, the composition comprises 10\hat{ }3 to 10\hat{ }12 colony forming units (cfu) per gram of bacteria. In some embodiments, the bacterial strains are grown in aerobic conditions. In some embodiments, the bacterial strains are grown without animal products. In some embodiments, the bacterial strains are grown in Tryptic Soy Broth (TSB). In some embodiments, the composition when stored in a sealed container placed at 20° C. retains at least about: 10\hat{ }4 cfu after 6 months, as measured by cfu counts. In some embodiments, the composition further comprises an excipient. In some embodiments, the composition further comprises a lyoprotectant. In some embodiments, the composition further comprises an emollient. In some embodiments, the composition further comprises or a salt thereof. In some embodiments, the bacterial strains when contacted with S. aureus cause a reduction in expression of at least one of the following genes: gmk, agr, psma, saeR, ccpA, and SigB in S. aureus, wherein the reduction in expression is measured by a fluorescence reporter assay. In some embodiments, the bacterial strains are present in an amount effective to suppress virulence of S. aureus as measured by a reduction in expression of at least one of the following genes: gmk, agr, psma, saeR, ccpA, and SigB in S. aureus wherein the reduction in expression is measured by a fluorescence reporter assay.

In some embodiments, a composition comprises: bacterial strains that are purified, wherein the bacterial strains comprise: a first strain, which comprises a 16s rRNA sequence with at least 97% sequence identity to SEQ ID NO: 5; a second strain, which comprises a 16s rRNA sequence with at least 97% sequence identity to SEQ ID NO: 3; and a third strain, which comprises a 16s rRNA sequence with at least 97% sequence identity to SEQ ID NO: 6; and wherein (a) the bacterial strains are lyophilized; or (b) the composition is formulated for delivery to a skin. In some embodiments, the first strain comprises the 16s rRNA sequence with at least 97% sequence identity over at least 1000 bases to SEQ ID NO: 5. In some embodiments, the second strain comprises the 16s rRNA sequence with at least 97% sequence identity over at least 1000 bases to SEQ ID NO: 3. In some embodiments, the third strain comprises the 16s rRNA sequence with at least 97% sequence identity over at least 1000 bases to SEQ ID NO: 6. In some embodiments, the first strain further comprises a sequence with at least 95% sequence identity to SEQ ID NO: 13. In some embodiments, the first strain further comprises having a sequence of SEQ ID NO: 13. In some embodiments, the second strain further comprises a sequence with at least 95% sequence identity to SEQ ID NO: 9, SEQ ID NO: 10, or both. In some embodiments, the second strain further comprises having a sequence of SEQ ID NO: 9, SEQ ID NO: 10, or both. In some embodiments, the third strain further comprises a sequence with at least 95% sequence identity to SEQ ID NO: 14. In some embodiments, the third strain further comprises having a sequence of SEQ ID NO: 14. In some embodiments, the composition is formulated as: a suspension, an emulsion, a cream, a lotion, a tincture, a gel, a foam, a powder, an ointment, a paste, or an oil. In some embodiments, the composition further comprising a fourth bacterial strain. In some embodiments, the composition comprises at least 10\hat{ }3 colony forming units (cfu) per gram of bacteria. In some embodiments, the composition comprises 10\hat{ }3 to 10\hat{ }12 colony forming units (cfu) per gram of bacteria. In some embodiments, the bacterial strains are grown in aerobic conditions. In some embodiments, the bacterial strains are grown without animal products. In some embodiments, the bacterial strains are grown in Tryptic Soy Broth (TSB). In some embodiments, the composition when stored in a sealed container placed at 20° C. retains at least about: 10\hat{ }4 cfu after 6 months, as measured by cfu counts. In some embodiments, the composition further comprises an excipient. In some embodiments, the composition further comprises a lyoprotectant. In some embodiments, the composition further comprises an emollient. In some embodiments, the composition further comprises or a salt thereof. In some embodiments, the bacterial strains when contacted with S. aureus cause a reduction in expression of at least one of the following genes: gmk, agr, psma, saeR, ccpA, and SigB in S. aureus, wherein the reduction in expression is measured by a fluorescence reporter assay. In some embodiments, the bacterial strains are present in an amount effective to suppress virulence of S. aureus as measured by a reduction in expression of at least one of the following genes: gmk, agr, psma, saeR, ccpA, and SigB in S. aureus wherein the reduction in expression is measured by a fluorescence reporter assay.

In some embodiments, a composition comprises: bacterial strains that are purified, wherein the bacterial strains comprise at least two of the following bacterial strains: a first strain, which comprises a 16s rRNA sequence with at least 97% sequence identity to SEQ ID NO: 4; a second strain, which comprises a 16s rRNA sequence with at least 97% sequence identity to SEQ ID NO: 1; a third strain, which comprises a 16s rRNA sequence with at least 97% sequence identity to SEQ ID NO: 2; a fourth strain, which comprises a 16s rRNA sequence with at least 97% sequence identity to SEQ ID NO: 5; a fifth strain, which comprises a 16s rRNA sequence with at least 97% sequence identity to SEQ ID NO: 6; and a sixth strain, which comprises a 16s rRNA sequence with at least 97% sequence identity to SEQ ID NO: 3; and wherein (a) the at least two bacterial strains are lyophilized; or (b) the composition is formulated for delivery to a skin. In some embodiments, the first strain further comprises a sequence with at least 95% sequence identity to SEQ ID NO: 11, SEQ ID NO: 12, or both. In some embodiments, the first strain further comprises having a sequence of SEQ ID NO: 11, SEQ ID NO: 12, or both. In some embodiments, the second strain further comprises a sequence with at least 95% sequence identity to SEQ ID NO: 15. In some embodiments, the second strain further having a sequence of SEQ ID NO: 15. In some embodiments, the third strain further comprises a sequence with at least 95% sequence identity to SEQ ID NO: 7, SEQ ID NO: 8, or both. In some embodiments, the third strain further comprises having a sequence of SEQ ID NO: 7, SEQ ID NO: 8, or both. In some embodiments, the fourth strain further comprises a sequence with at least 95% sequence identity to SEQ ID NO: 13. In some embodiments, the fourth strain further comprises having a sequence of SEQ ID NO: 13. In some embodiments, the fifth strain further comprises a sequence with at least 95% sequence identity to SEQ ID NO: 14. In some embodiments, the fifth strain further comprises having a sequence of SEQ ID NO: 14. In some embodiments, the sixth strain further comprises a sequence with at least 95% sequence identity to SEQ ID NO: 9, SEQ ID NO: 10, or both. In some embodiments, the sixth strain further comprises having a sequence of SEQ ID NO: 9, SEQ ID NO: 10, or both. In some embodiments, the composition is formulated as: a suspension, an emulsion, a cream, a lotion, a tincture, a gel, a foam, a powder, an ointment, a paste, or an oil. In some embodiments, the first strain comprises the 16s rRNA sequence with at least 97% sequence identity over at least 1000 bases to SEQ ID NO: 4. In some embodiments, the first strain comprises the 16s rRNA sequence with at least 97% sequence identity over at least 1000 bases to SEQ ID NO: 1. In some embodiments, the first strain comprises the 16s rRNA sequence with at least 97% sequence identity over at least 1000 bases to SEQ ID NO: 2. In some embodiments, the fourth strain comprises the 16s rRNA sequence with at least 97% sequence identity over at least 1000 bases to SEQ ID NO: 4. In some embodiments, the fifth strain comprises the 16s rRNA sequence with at least 97% sequence identity over at least 1000 bases to SEQ ID NO: 1. In some embodiments, the sixth strain comprises the 16s rRNA sequence with at least 97% sequence identity over at least 1000 bases to SEQ ID NO: 2. In some embodiments, the bacterial strains comprise bacterial strains the first strain and the second strain. In some embodiments, the bacterial strains comprise the fourth strain and the second strain. In some embodiments, the bacterial strains comprise the fourth strain and the sixth strain. In some embodiments, the bacterial strains comprise the first strain and the third strain. In some embodiments, the bacterial strains comprise the second strain and the third strain. In some embodiments, the bacterial strains comprise the fifth strain and the sixth strain. In some embodiments, the composition comprises at least 10\hat{ }3 colony forming units (cfu) per gram of bacteria. In some embodiments, the composition comprises 10\hat{ }3 to 10\hat{ }12 colony forming units (cfu) per gram of bacteria. In some embodiments, the bacterial strains are grown in aerobic conditions. In some embodiments, the bacterial strains are grown without animal products. In some embodiments, the bacterial strains are grown in Tryptic Soy Broth (TSB). In some embodiments, the composition when stored in a sealed container placed at 20° C. retains at least about: 10\hat{ }4 cfu after 6 months, as measured by cfu counts. In some embodiments, the composition further comprises an excipient. In some embodiments, the composition further comprises a lyoprotectant. In some embodiments, the composition further comprises an emollient. In some embodiments, the composition further comprises or a salt thereof. In some embodiments, the bacterial strains when contacted with S. aureus cause a reduction in expression of at least one of the following genes: gmk, agr, psma, saeR, ccpA, and SigB in S. aureus, wherein the reduction in expression is measured by a fluorescence reporter assay. In some embodiments, the bacterial strains are present in an amount effective to suppress virulence of S. aureus as measured by a reduction in expression of at least one of the following genes: gmk, agr, psma, saeR, ccpA, and SigB in S. aureus wherein the reduction in expression is measured by a fluorescence reporter assay.

In some embodiments, a composition comprises: a bacterial strain that is purified, wherein the bacterial strain comprises: a 16s rRNA sequence with at least 97% sequence identity to SEQ ID NO: 4; and wherein (a) the bacterial strain is lyophilized; or (b) the composition is formulated for delivery to a skin. In some embodiments, the bacterial strain comprises the 16s rRNA sequence with at least 97% sequence identity over at least 1000 bases to SEQ ID NO: 4. In some embodiments, the bacterial strain further comprises a sequence with at least 95% sequence identity to SEQ ID NO: 11, SEQ ID NO: 12, or both. In some embodiments, the bacterial strain further comprises having a sequence of SEQ ID NO: 11, SEQ ID NO: 12, or both. In some embodiments, the composition is formulated as: a suspension, an emulsion, a cream, a lotion, a tincture, a gel, a foam, a powder, an ointment, a paste, or an oil. In some embodiments, the composition further comprises a second bacterial strain. In some embodiments, the composition comprises at least 10\hat{ }3 colony forming units (cfu) per gram of bacteria. In some embodiments, the composition comprises 10\hat{ }3 to 10\hat{ }12 colony forming units (cfu) per gram of bacteria. In some embodiments, a bacterial strain is grown in aerobic conditions. In some embodiments, a bacterial strain is grown without animal products. In some embodiments, a bacterial strain is grown in Tryptic Soy Broth (TSB). In some embodiments, the composition when stored in a sealed container placed at 20° C. retains at least about: 10\hat{ }4 cfu after 6 months, as measured by cfu counts. In some embodiments, the composition further comprises an excipient. In some embodiments, the composition further comprises a lyoprotectant. In some embodiments, the composition further comprises an emollient. In some embodiments, the composition further comprises or a salt thereof. In some embodiments, the bacterial strains when contacted with S. aureus cause a reduction in expression of at least one of the following genes: gmk, agr, psma, saeR, ccpA, and SigB in S. aureus, wherein the reduction in expression is measured by a fluorescence reporter assay. In some embodiments, the bacterial strains are present in an amount effective to suppress virulence of S. aureus as measured by a reduction in expression of at least one of the following genes: gmk, agr, psma, saeR, ccpA, and SigB in S. aureus wherein the reduction in expression is measured by a fluorescence reporter assay.

In some embodiments, a composition comprises: a bacterial strain that is purified, wherein the bacterial strain comprises: a 16s rRNA sequence with at least 97% sequence identity to SEQ ID NO: 1; and wherein (a) the bacterial strain is lyophilized; or (b) the composition is formulated for delivery to a skin. In some embodiments, the bacterial strain comprises the 16s rRNA sequence with at least 97% sequence identity over at least 1000 bases to SEQ ID NO: 1. In some embodiments, the bacterial strain further comprises a sequence with at least 95% sequence identity to SEQ ID NO: 15. In some embodiments, the bacterial strain further comprises having a sequence of SEQ ID NO: 15. In some embodiments, the composition is formulated as: a suspension, an emulsion, a cream, a lotion, a tincture, a gel, a foam, a powder, an ointment, a paste, or an oil. In some embodiments, the composition further comprises a second bacterial strain. In some embodiments, the composition comprises at least 10\hat{ }3 colony forming units (cfu) per gram of bacteria. In some embodiments, the composition comprises 10\hat{ }3 to 10\hat{ }12 colony forming units (cfu) per gram of bacteria. In some embodiments, a bacterial strain is grown in aerobic conditions. In some embodiments, a bacterial strain is grown without animal products. In some embodiments, a bacterial strain is grown in Tryptic Soy Broth (TSB). In some embodiments, the composition when stored in a sealed container placed at 20° C. retains at least about: 10\hat{ }4 cfu after 6 months, as measured by cfu counts. In some embodiments, the composition further comprises an excipient. In some embodiments, the composition further comprises a lyoprotectant. In some embodiments, the composition further comprises an emollient. In some embodiments, the composition further comprises or a salt thereof. In some embodiments, the bacterial strains when contacted with S. aureus cause a reduction in expression of at least one of the following genes: gmk, agr, psma, saeR, ccpA, and SigB in S. aureus, wherein the reduction in expression is measured by a fluorescence reporter assay. In some embodiments, the bacterial strains are present in an amount effective to suppress virulence of S. aureus as measured by a reduction in expression of at least one of the following genes: gmk, agr, psma, saeR, ccpA, and SigB in S. aureus wherein the reduction in expression is measured by a fluorescence reporter assay.

In some embodiments, a composition comprises: a bacterial strain that is purified, wherein the bacterial strain comprises: a 16s rRNA sequence with at least 97% sequence identity to SEQ ID NO: 2; and wherein (a) the bacterial strain is lyophilized; or (b) the composition is formulated for delivery to a skin. In some embodiments, the bacterial strain comprises the 16s rRNA sequence with at least 97% sequence identity over at least 1000 bases to SEQ ID NO: 2. In some embodiments, the bacterial strain further comprises a sequence with at least 95% sequence identity to SEQ ID NO: 7, SEQ ID NO: 8, or both. In some embodiments, the bacterial strain further comprises having a sequence of SEQ ID NO: 7, SEQ ID NO: 8, or both. In some embodiments, the composition is formulated as: a suspension, an emulsion, a cream, a lotion, a tincture, a gel, a foam, a powder, an ointment, a paste, or an oil. In some embodiments, the composition further comprises a second bacterial strain. In some embodiments, the composition comprises at least 10\hat{ }3 colony forming units (cfu) per gram of bacteria. In some embodiments, the composition comprises 10\hat{ }3 to 10\hat{ }12 colony forming units (cfu) per gram of bacteria. In some embodiments, a bacterial strain is grown in aerobic conditions. In some embodiments, a bacterial strain is grown without animal products. In some embodiments, a bacterial strain is grown in Tryptic Soy Broth (TSB). In some embodiments, the composition when stored in a sealed container placed at 20° C. retains at least about: 10\hat{ }4 cfu after 6 months, as measured by cfu counts. In some embodiments, the composition further comprises an excipient. In some embodiments, the composition further comprises a lyoprotectant. In some embodiments, the composition further comprises an emollient. In some embodiments, the composition further comprises or a salt thereof. In some embodiments, the bacterial strains when contacted with S. aureus cause a reduction in expression of at least one of the following genes: gmk, agr, psma, saeR, ccpA, and SigB in S. aureus, wherein the reduction in expression is measured by a fluorescence reporter assay. In some embodiments, the bacterial strains are present in an amount effective to suppress virulence of S. aureus as measured by a reduction in expression of at least one of the following genes: gmk, agr, psma, saeR, ccpA, and SigB in S. aureus wherein the reduction in expression is measured by a fluorescence reporter assay.

In some embodiments, a composition comprises: a bacterial strain that is purified, wherein the bacterial strain comprises: a 16s rRNA sequence with at least 97% sequence identity to SEQ ID NO: 5; and wherein (a) the bacterial strain is lyophilized; or (b) the composition is formulated for delivery to a skin. In some embodiments, the bacterial strain comprises the 16s rRNA sequence with at least 97% sequence identity over at least 1000 bases to SEQ ID NO: 5. In some embodiments, the bacterial strain further comprises a sequence with at least 95% sequence identity to SEQ ID NO: 13. In some embodiments, the bacterial strain further comprises having a sequence of SEQ ID NO: 13. In some embodiments, the composition is formulated as: a suspension, an emulsion, a cream, a lotion, a tincture, a gel, a foam, a powder, an ointment, a paste, or an oil. In some embodiments, the composition further comprises a second bacterial strain. In some embodiments, the composition comprises at least 10\hat{ }3 colony forming units (cfu) per gram of bacteria. In some embodiments, the composition comprises 10\hat{ }3 to 10\hat{ }12 colony forming units (cfu) per gram of bacteria. In some embodiments, a bacterial strain is grown in aerobic conditions. In some embodiments, a bacterial strain is grown without animal products. In some embodiments, a bacterial strain is grown in Tryptic Soy Broth (TSB). In some embodiments, the composition when stored in a sealed container placed at 20° C. retains at least about: 10\hat{ }4 cfu after 6 months, as measured by cfu counts. In some embodiments, the composition further comprises an excipient. In some embodiments, the composition further comprises a lyoprotectant. In some embodiments, the composition further comprises an emollient. In some embodiments, the composition further comprises or a salt thereof. In some embodiments, the bacterial strains when contacted with S. aureus cause a reduction in expression of at least one of the following genes: gmk, agr, psma, saeR, ccpA, and SigB in S. aureus, wherein the reduction in expression is measured by a fluorescence reporter assay. In some embodiments, the bacterial strains are present in an amount effective to suppress virulence of S. aureus as measured by a reduction in expression of at least one of the following genes: gmk, agr, psma, saeR, ccpA, and SigB in S. aureus wherein the reduction in expression is measured by a fluorescence reporter assay.

In some embodiments, a composition comprises: a bacterial strain that is purified, wherein the bacterial strain comprises: a 16s rRNA sequence with at least 97% sequence identity to SEQ ID NO: 6; and wherein (a) the bacterial strain is lyophilized; or (b) the composition is formulated for delivery to a skin. In some embodiments, the bacterial strain comprises the 16s rRNA sequence with at least 97% sequence identity over at least 1000 bases to SEQ ID NO: 6. In some embodiments, the bacterial strain further comprises a sequence with at least 95% sequence identity to SEQ ID NO: 14. In some embodiments, the bacterial strain further comprises having a sequence of SEQ ID NO: 14. In some embodiments, the composition is formulated as: a suspension, an emulsion, a cream, a lotion, a tincture, a gel, a foam, a powder, an ointment, a paste, or an oil. In some embodiments, the composition further comprises a second bacterial strain. In some embodiments, the composition comprises at least 10\hat{ }3 colony forming units (cfu) per gram of bacteria. In some embodiments, the composition comprises 10\hat{ }3 to 10\hat{ }12 colony forming units (cfu) per gram of bacteria. In some embodiments, a bacterial strain is grown in aerobic conditions. In some embodiments, a bacterial strain is grown without animal products. In some embodiments, a bacterial strain is grown in Tryptic Soy Broth (TSB). In some embodiments, the composition when stored in a sealed container placed at 20° C. retains at least about: 10\hat{ }4 cfu after 6 months, as measured by cfu counts. In some embodiments, the composition further comprises an excipient. In some embodiments, the composition further comprises a lyoprotectant. In some embodiments, the composition further comprises an emollient. In some embodiments, the composition further comprises or a salt thereof. In some embodiments, the bacterial strains when contacted with S. aureus cause a reduction in expression of at least one of the following genes: gmk, agr, psma, saeR, ccpA, and SigB in S. aureus, wherein the reduction in expression is measured by a fluorescence reporter assay. In some embodiments, the bacterial strains are present in an amount effective to suppress virulence of S. aureus as measured by a reduction in expression of at least one of the following genes: gmk, agr, psma, saeR, ccpA, and SigB in S. aureus wherein the reduction in expression is measured by a fluorescence reporter assay.

In some embodiments, a composition comprises: a bacterial strain that is purified, wherein the bacterial strain comprises: a 16s rRNA sequence with at least 97% sequence identity to SEQ ID NO: 3; and wherein (a) the bacterial strain is lyophilized; or (b) the composition is formulated for delivery to a skin. In some embodiments, the bacterial strain comprises the 16s rRNA sequence with at least 97% sequence identity over at least 1000 bases to SEQ ID NO: 3. In some embodiments, the bacterial strain further comprises a sequence with at least 95% sequence identity to SEQ ID NO: 9, SEQ ID NO: 10, or both. In some embodiments, the bacterial strain further comprises having a sequence of SEQ ID NO: 9, SEQ ID NO: 10, or both. In some embodiments, the composition is formulated as: a suspension, an emulsion, a cream, a lotion, a tincture, a gel, a foam, a powder, an ointment, a paste, or an oil. In some embodiments, the composition further comprises a second bacterial strain. In some embodiments, the composition comprises at least 10\hat{ }3 colony forming units (cfu) per gram of bacteria. In some embodiments, the composition comprises 10\hat{ }3 to 10\hat{ }12 colony forming units (cfu) per gram of bacteria. In some embodiments, a bacterial strain is grown in aerobic conditions. In some embodiments, a bacterial strain is grown without animal products. In some embodiments, a bacterial strain is grown in Tryptic Soy Broth (TSB). In some embodiments, the composition when stored in a sealed container placed at 20° C. retains at least about: 10\hat{ }4 cfu after 6 months, as measured by cfu counts. In some embodiments, the composition further comprises an excipient. In some embodiments, the composition further comprises a lyoprotectant. In some embodiments, the composition further comprises an emollient. In some embodiments, the composition further comprises or a salt thereof. In some embodiments, the bacterial strains when contacted with S. aureus cause a reduction in expression of at least one of the following genes: gmk, agr, psma, saeR, ccpA, and SigB in S. aureus, wherein the reduction in expression is measured by a fluorescence reporter assay. In some embodiments, the bacterial strains are present in an amount effective to suppress virulence of S. aureus as measured by a reduction in expression of at least one of the following genes: gmk, agr, psma, saeR, ccpA, and SigB in S. aureus wherein the reduction in expression is measured by a fluorescence reporter assay.

Also disclosed herein are methods of administering the composition described above. In some embodiments, a method comprises administering an amount sufficient to treat a disease selected from the group consisting of: atopic dermatitis, seborrheic dermatitis, inflammation, eczema, psoriasis, rosacea, mycoses, dermatophytosis, folliculitis, acne, alopecia, vitiligo, dandruff, chronic wound, skin ulcer, Netherton syndrome, hidradenitis suppurativa, sycosis vulgaris, staphylococcal scalded skin syndrome, impetigo, ecthyma, cellulitis, carbuncle, furuncle, and abscess. In some embodiments, a method comprises administering an amount sufficient to reduce symptoms associated with atopic dermatitis. In some embodiments, a method comprises administering the composition to a subject who has an eczema prone microbiome prior to the administration. In some embodiments, a method comprises administering an amount sufficient to reduce symptoms associated with dry skin.

Also disclosed herein are methods of administering a composition described above. In some embodiments, the method comprises administering an amount sufficient for a reduction of incidence of a condition associated with inflammation and wherein the condition associated with inflammation comprises an itch, a rash, a redness, a pain, a swelling, a blistering, or a scaling. In some embodiments, a method comprises administering an amount sufficient to suppress virulence of S. aureus as measured by a reduction in expression of at least one of the following genes: gmk, agr, psma, saeR, ccpA, and SigB in S. aureus, wherein the reduction in expression is measured by a fluorescence reporter assay. In some embodiments, the method of administering is topical administration.

Also disclosed herein are compositions, such as the compositions disclosed above, for use in the treatment of a skin condition. Disclosed herein are compositions for use in the treatment of inflammation.

Also disclosed herein are method for treatment of inflammation, comprising: topically administering a composition comprising a bacterial strain, which comprises a 16s rRNA sequence with at least 97% sequence identity to SEQ ID NO: 1 to a subject in need thereof, wherein the bacterial strain is purified, and wherein the bacterial strain is present in an amount sufficient for treatment of inflammation.

Also disclosed herein are methods for reducing growth of Staphylococcus aureus on skin of a subject, comprising: topically administering to a skin of the subject a pharmaceutical composition comprising: a first bacterial strain, which comprises a 16s rRNA sequence with at least 97% sequence identity to SEQ ID NO: 5; a second bacterial strain, which comprises a 16s rRNA sequence with at least 97% sequence identity to SEQ ID NO: 1; and a third bacterial strain, which comprises a 16s rRNA sequence with at least 97% sequence identity to SEQ ID NO: 2, wherein the bacterial strains are purified, and wherein the bacterial strains are viable, and present in an amount sufficient for reduction of S. aureus on a skin of the subject in need thereof.

Also disclosed herein are methods for reduction of symptoms associated with atopic dermatitis, comprising: topically administering a composition comprising a bacterial strain, which comprises a 16s rRNA sequence with at least 97% sequence identity to SEQ ID NO: 5, to a subject in need thereof, wherein the bacterial strain is purified, and wherein the bacterial strain is present in an amount sufficient for the reduction of symptoms associated with atopic dermatitis.

Also disclosed herein are methods for reducing an incidence of a condition associated with inflammation comprising: topically administering a composition comprising: a first bacterial strain, which comprises a 16s rRNA sequence with at least 97% sequence identity to SEQ ID NO: 1; and a second bacterial strain, which comprises a 16s rRNA sequence with at least 97% sequence identity to SEQ ID NO: 6, to a subject in need thereof, wherein the bacterial strains are purified, wherein the bacterial strains are present in an amount sufficient for a reduction of the incidence of a condition associated with inflammation and wherein the condition associated with inflammation comprises an itch, a rash, a redness, a pain, a swelling, a blistering, or a scaling.

Also disclosed herein are methods of administering the compositions described above. In some embodiments, a method can comprise administering an amount sufficient to treat a condition selected from the group consisting of: pruritis, an aesthetic condition, and body odor. In some embodiments, the aesthetic condition comprises wrinkles or appearance of aging.

Also disclosed herein are methods for reducing growth of Staphylococcus aureus on skin of a subject, comprising: topically administering to a skin of the subject, a composition comprising: a first bacterial strain, which comprises a 16s rRNA sequence with at least 97% sequence identity to SEQ ID NO: 5; a second bacterial strain, which comprises a 16s rRNA sequence with at least 97% sequence identity to SEQ ID NO: 1; and a third bacterial strain, which comprises a 16s rRNA sequence with at least 97% sequence identity to SEQ ID NO: 2, wherein the bacterial strains are purified, and wherein the bacterial strains are viable, and present in an amount sufficient for enhancing skin appearance of the subject. In some embodiments, the enhancing skin appearance of the subject comprises reducing wrinkles, decreasing lesion size, decreasing pore size, decreasing pore density, decreasing wrinkle occurrence, decreasing wrinkle depth, decreasing age spots, or increasing skin elasticity.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1, is a pie chart that summaries the number of strain combinations tested that inhibited different S. aureus genes. FIG. 1 shows percent of two strain combinations that inhibit expression of agr and psmA (left) or inhibit expression agr, psmA, and sigB (right) of Staphylococcus aureus. Combinations of strains that inhibit multiple functions of S. aureus are rare.

FIG. 2, is a bar graph that plots percent activity of S. aureus gene expression by a fluorescent reporter. The X-axis shows the strains in the combinatorial screening assay. The Y-axis shows the percent activity (from 0-100%) as compared to the S. aureus monoculture. FIG. 2 shows inhibition of different S. aureus promoter-reporter constructs when reporter strains of S. aureus were mixed with single strain, two strains or three strains of Strain jl.83, Strain jl.27 and Strain jl.77.

FIG. 3, is a bar graph that plots percent activity of S. aureus gene expression by a fluorescent reporter. The X-axis shows the strains in the combinatorial screening assay. The Y-axis shows the percent activity (from 0-100%) as compared to the S. aureus monoculture. FIG. 3 shows inhibition of different S. aureus promoter-reporter constructs when reporter strains of S. aureus were mixed with single strain, two strains or three strains of Strain jl.21, Strain jl.68, and Strain jl.121.

FIG. 4, is a bar graph that plots percent activity of S. aureus gene expression by a fluorescent reporter. The X-axis shows the strains in the combinatorial screening assay. The Y-axis shows the percent activity (from 0-100%) as compared to the S. aureus monoculture. FIG. 4 shows inhibition of different S. aureus promoter-reporter constructs when the reporter strains of S. aureus were mixed with single strain, two strains or three strains of Strain jl.27, Strain jl.68, and Strain jl.121.

FIG. 5, is a bar graph that plots percent change of S. aureus gene expression by a fluorescent reporter in the three-strain mixture of Strain jl.83, Strain jl.27 and Strain jl.77 as compared to S. aureus monoculture in different environmental conditions. The Y-axis shows the percent change as compared to the S. aureus monoculture. The X-axis shows the environmental condition that was tested. FIG. 5 shows inhibition of different S. aureus promoter-reporter constructs (agr, psmA, GMK) when the reporter strains of S. aureus were mixed with the three-strain mixture of Strain jl.83, Strain jl.27 and Strain jl.77 in the presence of additional environmental conditions (e.g., different nutrients). This data shows the strain combination retained S. aureus inhibition function across different nutrients.

FIG. 6 is a bar graph that plots percent change of S. aureus gene expression by a fluorescent reporter in the three-strain mixture of Strain jl.21, Strain jl.68, and Strain jl.121 as compared to S. aureus monoculture in different environmental conditions. The Y-axis shows the percent change as compared to the S. aureus monoculture. The X-axis shows the environmental condition that was tested. FIG. 6 shows inhibition of different S. aureus promoter-reporter constructs (agr, psmA, GMK) when the reporter strains of S. aureus were mixed with the three-strain mixture of Strain jl.21, Strain jl.68, and Strain jl.121 in the presence of additional environmental conditions (e.g., different nutrients). This data shows the strain combination retained S. aureus inhibition function across different nutrients.

FIG. 7, is a bar graph that plots percent change of S. aureus gene expression by a fluorescent reporter in the three-strain mixture of Strain jl.27, Strain jl.68, and Strain jl.121 as compared to S. aureus monoculture in different environmental conditions. The Y-axis shows the percent change as compared to the S. aureus monoculture. The X-axis shows the environmental condition that was tested. FIG. 7 shows inhibition of different S. aureus promoter-reporter constructs (agr, psmA, GMK) when the reporter strains of S. aureus were mixed with the three-strain mixture of Strain jl.27, Strain jl.68, and Strain jl.121 in the presence of additional environmental conditions (e.g., different nutrients). This data shows the strain combination retained S. aureus inhibition function across different nutrients.

FIG. 8, is a bar graph that plots percent activity of S. aureus gene expression by a fluorescent reporter in different growth conditions. The Y-axis shows the percent activity as compared to the S. aureus monoculture. The X-axis shows the expression of S. aureus agr (quorum sensing), psmA (toxin production), and GMK (metabolic function). FIG. 8 shows inhibition of different S. aureus promoter-reporter constructs (agr, psmA, GMK) when the reporter strains of S. aureus were mixed with the three-strain mixture of Strain jl.83, Strain jl.27 and Strain jl.77 in the presence of thiamine. The presence of thiamine enhances the inhibition of S. aureus by the three-strain mixture.

FIG. 9, is a bar graph that plots max colony-forming unit (cfu)/ml of isolated strains in different media. The Y-axis shows the cfu/mL observed across the tested media. The X-axis indicates the strain tested and the animal-free media formulation used for the strain tested. FIG. 9 shows growth of different skin isolates in animal free media compared to animal-based medium. This figure shows increased growth for most isolated strains in animal-free media.

FIG. 10, is a bar graph that plots percent of growth of mixed cultures as compared to S. aureus monoculture. The Y-axis shows the effect of growth inhibition (%) by the strain combination. The X-axis shows the growth of different S. aureus strains in the presence of an ensemble (e.g., the three-strain combination) or the S. aureus strain alone. FIG. 10 shows inhibition of different S. aureus strains when grown with the strains Strain jl.83, Strain jl.27 and Strain jl.77. This figure shows the combination of the isolates are able to inhibit growth of numerous S. aureus isolates.

FIG. 11, is a bar graph that plots percent growth of mixed cultures as compared to S. aureus monoculture. The Y-axis shows the effect of growth inhibition (%) by the strain combination. The X-axis shows the growth of different S. aureus strains in the presence of an ensemble (e.g., the three-strain combination) or the S. aureus strain alone. FIG. 11 shows inhibition of different S. aureus strains when grown with the strains Strain jl.21, Strain jl.68, and Strain jl.121. This figure shows the combination of the isolates are able to inhibit growth of numerous S. aureus isolates.

FIG. 12, is a bar graph that plots percent growth of mixed cultures as compared to S. aureus monoculture. The Y-axis shows the effect of growth inhibition (%) by the strain combination. The X-axis shows the growth of different S. aureus strains in the presence of an ensemble (e.g., the three-strain combination) or the S. aureus strain alone. FIG. 12 shows inhibition of different S. aureus strains when grown with the strains Strain jl.27, Strain jl.68, and Strain jl.121. This figure shows the combination of the isolates are able to inhibit growth of numerous S. aureus isolates.

FIG. 13, is a data plot that plots the log₂ (coculture/monoculture) change of S. aureus gene expression by a fluorescent reporter in different mixtures with Strain jl.27. The Y-axis shows the log₂ expression change of the coculture/a S. aureus promoter-reporter monoculture. The X-axis shows the gene promoters tested. FIG. 13 shows inhibition of different S. aureus promoter-reporter constructs when the reporter strains of S. aureus were mixed with strain combinations of Strain jl.27.

FIG. 14, is a bar graph that plots the percent S. aureus behavior (i.e. expression) of S. aureus genes by fluorescent reporters of agr, psmA, and GMK. The expression plotted is shown as compared to S. aureus monoculture expression of the reporters. Different strain combinations of Strain jl.121, Strain jl.21, and Strain jl.68 were tested in the presence of up to 7 microbe communities for their ability to reduce S. aureus gene expression. The Y-axis shows the percent expression of the reporter as compared to the S. aureus monoculture. The X-axis shows the strain combinations that were tested. The data shows the 3-species combination of Strain jl.121, Strain jl.21, and Strain jl.68 was typically more robust at reducing gene expression than their subsets (e.g., a single strain or two strain combination), and were able to reduce S. aureus gene expression in the presence of additional microbes.

FIG. 15, is a bar graph that plots the percent S. aureus behavior (i.e. expression) of S. aureus genes by fluorescent reporters of agr, psmA, and GMK. The expression plotted is shown as compared to S. aureus monoculture expression of the reporters. Different strain combinations of Strain jl.121, Strain jl.27, and Strain jl.68 were tested in the presence of up to 7 microbe communities for their ability to reduce S. aureus gene expression. The Y-axis shows the percent expression of the reporter as compared to the S. aureus monoculture. The X-axis shows the strain combinations that were tested. The data shows the 3-species combination of Strain jl.121, Strain jl.27, and Strain jl.68 was typically more robust at reducing gene expression than their subsets (e.g., a single strain or two strain combination), and were able to reduce S. aureus gene expression in the presence of additional microbes.

FIG. 16, is a bar graph that plots the percent S. aureus behavior (i.e. expression) of S. aureus genes by fluorescent reporters of agr, psmA, and GMK. The expression plotted is shown as compared to S. aureus monoculture expression of the reporters. Different strain combinations of Strain jl.27, Strain jl.77, and Strain jl.83 were tested in the presence of up to 7 microbe communities for their ability to reduce S. aureus gene expression. The Y-axis shows the percent expression of the reporter as compared to the S. aureus monoculture. The X-axis shows the strain combinations that were tested. The data shows the 3-species combination of Strain jl.27, Strain jl.77, and Strain jl.83 was typically more robust at reducing gene expression than their subsets (e.g., a single strain or two strain combination), and were able to reduce S. aureus gene expression in the presence of additional microbes.

FIGS. 17A-17B, depict images of agar plates containing Strain jl.68 whole cells plated on a lawn of S. aureus cells. The S. aureus cells contained reporters for expression of agr (quorum sensing) and GMK (metabolism). The expression of agr and GMK was affected by the addition of Strain jl.68. FIG. 17A shows a zoomed-out picture of the plate. FIG. 17B shows a zoomed-in picture of the plate.

FIGS. 18A-18F, depict bar graphs that show expression of S. aureus agr and GMK reporters in S. aureus cells and the CFU of S. aureus cells in monoculture or after being mixed with different strains of bacteria. FIG. 18A shows the CFU of S. aureus and the expression of GMK and agr in a S. aureus monoculture. FIG. 18B shows the CFU of S. aureus and the expression of GMK and agr in 1:1 coculture with a WT strain of S. aureus (not having the agr/GMK reporter). FIG. 18C shows the CFU of S. aureus and the expression of GMK and agr in 1:1 coculture with the three-strain mix of Strain jl.27, Strain jl.68, and Strain jl.77. FIG. 18D shows the CFU of S. aureus and the expression of GMK and agr in 1:1 coculture with the single Strain jl.68. FIG. 18E shows the CFU of S. aureus and the expression of GMK and agr in culture with supernatant from a three-strain mix of Strain jl.27, Strain jl.68, and Strain jl.77. FIG. 18F shows the CFU of S. aureus and the expression of GMK and agr in culture with supernatant from Strain jl.68.

FIGS. 19A-19B, depict bar graphs that show metabolic activity and quorum sensing activity in different densities of S. aureus cultures. Supernatant (10%) from Strain jl.68 or PBS (control) was added to the different concentrations of S. aureus. FIG. 19A shows the expression of GMK after the addition of supernatant. FIG. 19B shows the expression of agr after the addition of supernatant.

FIG. 20, depict images of agar plates that show different concentrations (0%, 5%, and 10%) of supernatant from Strain jl.68 embedded in TSB agar plates with 10-fold dilutions of S. aureus plated on top of the agar. The CFU/ml is the amount of S. aureus present on the plates after incubation.

### DETAILED DESCRIPTION OF THE INVENTION

Provided herein are compositions, methods, kits and devices for the treatment of skin diseases. Furthermore, provided herein are (1) mixtures of bacteria (2) excipients, dosage forms and routes of administration for such mixtures, (3) and conditions for treatment with such bacterial mixtures.

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included, unless the context clearly dictates otherwise.

The terminology used herein is for the purpose of describing particular instances only and is not intended to be limiting of any embodiment. As used herein, the singular forms “a,” “an” and “the” are intended to include the plural forms as well, unless the context clearly indicates otherwise. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The term “subject” as used herein includes human and non-human mammals, including for example: a primate, cow, horse, pig, sheep, goat, dog, cat, or rodent, capable of being colonized by other organisms.

In some embodiments, provided herein are compositions which include bacteria having a percent identity based on 16S rRNA bacterial genetic sequence, a hypervariable region of the 16S rRNA, or whole genome comparison to a reference strain. Typically, comparison of the 16S rRNA bacterial genetic sequence allows a strain to be identified as within the same species as another strain by comparing sequences with known bacterial DNA sequences using NCBI BLAST search. The level of identity in relation to a nucleotide sequence may be determined for at least 20 contiguous nucleotides, for at least 30 contiguous nucleotides, for at least at least 40 contiguous nucleotides, for at least 50 contiguous nucleotides, for at least 60 contiguous nucleotides, for at least 100 contiguous nucleotides, for at least 200 contiguous nucleotides, for at least 300 contiguous nucleotides, for at least 400 contiguous nucleotides, for at least 500 contiguous nucleotides, for at least 600 contiguous nucleotides, for at least 700 contiguous nucleotides, for at least 800 contiguous nucleotides, for at least 900 contiguous nucleotides, for at least 1000 contiguous nucleotides, for at least 1100 contiguous nucleotides, for at least 1200 contiguous nucleotides, for at least 1300 contiguous nucleotides, for at least 1400 contiguous nucleotides, or for at least 1500 contiguous nucleotides. In some embodiments, the level of identity in relation to a nucleotide sequence is determined for the entire sequence searched. Percent identity may be at least 70%, 80%, 85%, 90%, 91%, 92%, 93%, 94%, 95%, 96%, 97%, 98%, 99%, or 100% to a reference bacterial 16S rRNA sequence, 16S rRNA V4 region sequence, or whole genome sequence. Percent identity may be at least 70%, 80%, 85%, 90%, 91%, 92%, 93%, 94%, 95%, 96%, 97%, 98%, 99%, or 100% to a reference bacteria 16S rRNA: V1 region, V2 region, V3 region, V5 region, V6 region, V7 region, V8 region or V9 region sequence. Percent identity may be at least 70%, 80%, 85%, 90%, 91%, 92%, 93%, 94%, 95%, 96%, 97%, 98%, 99%, or 100% to a reference bacterial sequence.

As used herein, a substance is “pure” or “substantially pure” if it is substantially free of other components of the sample source from which the substance is obtained. The terms “purify,” “purifying” and “purified”, when applied to a bacterium or bacteria, can refer to a bacterium that has been separated from at least some of the components with which it was associated either when initially produced or generated, or during any time after its initial production. A bacterium or a bacterial population may be considered purified if it is isolated at or after production, such as from a material or environment containing the bacterium or bacterial population, or by passage through culture. A purified bacterium or bacterial population may contain other materials up to at least about 10%, about 20%, about 30%, about 40%, about 50%, about 60%, about 70%, about 80%, about 90%, or above about 90% and still be considered purified. A purified bacterium or bacterial population may contain a single type of strain present in at least about 5, 10, 15, 20, 25,30, 35, 40, 45, 50% or more of the bacteria present in a composition and still be considered purified.

### Bacterial Mixtures

The skin of a human provides many environments for the microbiome. In healthy individuals, both the microbiome of the skin and the host environment of the skin are important for maintaining skin health. However, dysbiosis of the skin microbiome and replacement of commensal bacterial species with pathogenic bacterial species can cause disease. To this end, perturbation of a damaged skin microbiome with bacterial isolates from healthy individuals can restore function and diversity of a skin microbiome. For example, restoration and/or modification of a damaged microbiome can be used to treat a disease. Compositions described herein are used for treatment of a damaged skin microbiome and contain bacterial species that are purified. In some embodiments, the bacterial species comprise at least one, two, or three of the following bacterial species: Bacillus velezensis, Bacillus wiedmannii, Lysinibacillus macrolides, Bacillus amyloliquefaciens, Staphylococcus caprae, and Bacillus tropicus. In some embodiments, a composition comprises at least one, two, or three of the following bacterial species: Bacillus cereus, Staphylococcus epidermidis, Bacillus nakamurai, Bacillus vallismortis, Lysinibacillus boronitolerans, Bacillus paranthracis, Bacillus thuringiensis, Psychrobacillus sp. INOP01, Lysinibacillus sp. FN11, Bacillus sp. Lzh-5, or Staphylococcus capitis. The bacterial species are lyophilized and/or formulated for delivery to the skin. A composition can be formulated as a suspension, an emulsion, a cream, a lotion, a tincture, a gel, a foam, a powder, an ointment, a paste, or an oil. Administration of the bacterial species are used to treat inflammation of the skin, a skin disorder, such as atopic dermatitis, or a symptom associated with inflammation of the skin or a skin disorder.

Additionally, compositions described herein are used for treatment of a damaged skin microbiome and contain bacterial strains that are purified. In some embodiments, the bacterial strains comprise at least one, two, or three of the following bacterial strains: Strain jl.121, Strain jl.21, Strain jl.27, Strain jl.68, Strain jl.83, or Strain jl.77. The bacterial strains are lyophilized and/or formulated for delivery to the skin. A composition can be formulated as a suspension, an emulsion, a cream, a lotion, a tincture, a gel, a foam, a powder, an ointment, a paste, or an oil. Administration of the bacterial strains are used to treat inflammation of the skin, a skin disorder, such as atopic dermatitis, or a symptom associated with inflammation of the skin or a skin disorder

Bacteria described herein and mixtures of the bacteria described herein are used to treat or inhibit, reduce, and/or eliminate pathogenic bacteria from colonizing or inducing inflammation of the skin. Bacteria described herein and mixtures of the bacteria described herein are used to suppress the growth of pathogenic bacteria. Bacteria described herein are used to modulate the expression of a gene in a pathogenic microorganism. Bacteria described herein are used to modulate the expression of a protein in a pathogenic microorganism. Bacteria described herein are isolated from the skin of a healthy individual. In some embodiments, bacteria described herein are purified. For example, a bacterial isolate described herein can be purified by one or more passages on an agar medium. In some cases, a healthy individual is a subject without one or more diseases of the skin. In some cases, bacteria are isolated from the epidermis of the skin. In some cases, bacteria are isolated from the skin of a foot, a leg, an arm, a torso, a hand, a groin, an armpit, a back, a neck, a head, an ear, a nose, a face or any region of the body covered in skin. In some embodiments, bacteria described herein are in a composition. In some embodiments, bacteria described herein are administered to treat a disease. In some cases, compositions described herein are lyophilized. In some cases, compositions described herein are formulated for delivery to a skin.

In some embodiments, a composition comprises live bacteria. In some cases, a live bacterium comprises a bacterium that retains membrane stability. In some cases, a live bacterium comprises a bacterium that is capable of transcription and translation. In some cases, a live bacterium comprises a bacterium that is capable of cell division. In some cases, live bacteria are determined by a culture dependent or a culture independent technique. In some cases, live bacteria comprise an individual or a group of bacteria that can produce a colony-forming unit (cfu) when plated on stable growth media. In some embodiments, live and/or dead bacteria are determined by imaging, for example with a live/dead stain. In some cases, a viability PCR based method can be used to determine live bacteria. In some cases, a metabolomic assay is used to determine live bacteria.

In some embodiments, bacteria described herein are grown in aerobic conditions. In some cases, bacteria described herein are grown in anaerobic conditions. In some cases, bacteria described herein can be an obligate aerobe, an obligate anaerobe, a facultative anaerobe, a microaerophile, or an aerotolerant organism and can be grown in any condition applicable for growth, (e.g., 0-40% O₂, and/or 0-40% CO₂). Bacteria described herein are grown at room temperature or in an incubator. For example, bacteria described herein can be grown in any temperature conducive for growth. In some instances, bacteria described herein are grown at about 1° C. to 50° C. In some instances, bacteria described herein are grown at 30° C. In some instances, bacteria described herein are grown at 37° C. Bacteria described herein are grown in liquid culture or on a solid media, such as agar supplemented with nutrients. In some cases, bacteria are grown in a medium with or without animal products. In some cases, bacteria are grown in a medium without animal products. In some instances, bacteria are grown in Tryptic Soy Broth (TSB).

In some cases, bacteria and/or a composition described herein are stored in a container. In some cases, a container is glass, plastic, metal, or any solid material. In some cases, a container is comprised in a kit. In some instances, when a sealed container containing bacteria or a composition described herein is placed at about: −80° C., −79° C., −78° C., −77° C., −76° C., −75° C., −74° C., −73° C., −72° C., −71° C., −70° C., −69° C., −68° C., −67° C., −66° C., −65° C., −64° C., −63° C., −62° C., −61° C., −60° C., −59° C., −58° C., −57° C., −56° C., −55° C., −54° C., −53° C., −52° C., −51° C., −50° C., −49° C., −48° C., −47° C., −46° C., −45° C., −44° C., −43° C., −42° C., −41° C., −40° C., −39° C., −38° C., −37° C., −36° C., −35° C., −34° C., −33° C., −32° C., −31° C., −30° C., −29° C., −28° C., −27° C., −26° C., −25° C., −24° C., −23° C., −22° C., −21° C., −20° C., −19° C., −18° C., −17° C., −16° C., −15° C., −14° C., −13° C., −12° C., −11° C., −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., or 38° C., the bacteria can retain greater than about: 0.1%, 1%, 10%, 20%, 30%, 40%, 50%, 60%, 70%, 80%, 90% or 99% culture viability after 3 months, 6 months, or 12 months, as measured by cfu counts. In some instances, when a sealed container containing bacteria or a composition described herein is placed at about: −80° C., −79° C., −78° C., −77° C., −76° C., −75° C., −74° C., −73° C., −72° C., −71° C., −70° C., −69° C., −68° C., −67° C., −66° C., −65° C., −64° C., −63° C., −62° C., −61° C., −60° C., −59° C., −58° C., −57° C., −56° C., −55° C., −54° C., −53° C., −52° C., −51° C., −50° C., −49° C., −48° C., −47° C., −46° C., −45° C., −44°° C., −43° C., −42° C., −41° C., −40° C., −39° C., −38° C., −37° C., −36° C., −35° C., −34° C., −33° C., −32° C., −31° C., −30° C., −29° C., −28° C., −27° C., −26° C., −25° C., −24° C., −23° C., −22° C., −21° C., −20° C., −19° C., −18° C., −17° C., −16° C., −15° C., −14° C., −13° C., −12° C., −11° C., −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., or 38° C., the bacteria can retain greater than about: about 10³ cfu, about 10⁴ cfu, about 10⁵ cfu, about 10⁶ cfu, about 10⁷ cfu, about 10⁸ cfu, about 10⁹ cfu, about 10¹⁰ cfu, about 10¹¹ cfu, about 10¹² cfu after 3 months, 6 months, or 12 months, as measured by cfu counts. In some cases, the compositions disclosed herein are stable in a freezer (e.g., −80° C. to about −20° C.), a refrigerator (e.g. 4° C.), or at room temperature.

In some embodiments, provided herein are compositions of bacterial species. In some cases, a composition described herein comprises one or more bacterial species. For example, a composition described herein comprises 2, 3, 4, 5, 6, 7, 8, 9, 10 or more bacterial species. In some cases, a composition described herein comprises one bacterial species. In some cases, a composition described herein comprises two bacterial species. In some cases, a composition described herein comprises three bacterial species. In some cases, a composition described herein comprises four bacterial species. Provided herein are mixtures of bacteria comprising one or more a bacterial species of Table 1. In some embodiments, a composition described herein comprises 1, 2, 3, 4, 5, 6, 7, 8, 9, 10 or more bacterial species in Table 1.

TABLE 1
Bacterial Species

Species name
Staphylococcus capitis
Staphylococcus caprae
Staphylococcus cohnii
Staphylococcus epidermidis
Staphylococcus intermedius
Staphylococcus lugdunensis
Staphylococcus pasteuri
Staphylococcus saprophyticus
Staphylococcus warneri
Bacillus velezensis
Bacillus amyloliquefaciens
Bacillus cecembensis
Bacillus cereus
Bacillus sp. Lzh-5
Bacillus sp. KbaL1
Bacillus sp. SJ-10
Bacillus mediterraneensis
Bacillus mycoides
Bacillus nakamurai
Bacillus thuringiensis
Bacillus tropicus
Bacillus paranthracis
Bacillus vallismortis
Micrococcus aloeverae
Psychrobacillus sp. INOP01
Paenibacillus lentimorbus
Paenibacillus elgii
Bacillus wiedmannii
Brevibacillus parabrevis
Dermacoccus nishinomiyaensis
Kocuria marina
Lysinibacillus boronitolerans
Lysinibacillus macroides
Lysinibacillus sp. FN11
Lysinibacillus sp. UBA5994
Lysinibacillus sp. PB300
Lysinibacillus sp. YS11
Lysinibacillus boronitolerans
Lysinibacillus fusiformis
Macrococcus goetzii

In some cases, a composition described herein comprises one or more bacterial strains. For example, a composition described herein comprises 2, 3, 4, 5, 6, 7, 8, 9, 10 or more bacterial strains. In some cases, a composition described herein comprises one bacterial strain. In some cases, a composition described herein comprises two bacterial strains. In some cases, a composition described herein comprises three bacterial strains. In some cases, a composition described herein comprises four bacterial strains. Provided herein are mixtures of bacteria comprising a one or more bacterial strains of Table 2. In some embodiments, a composition described herein comprises 1, 2, 3, 4, 5, 6, 7, 8, 9, 10 or more bacterial strains in Table 2.

TABLE 2
Bacterial Strains

| Strain Number | Strain Name |
|---|---|
| Strain 1 | Strain jl.19 |
| Strain 2 | Strain jl.39 |
| Strain 3 | Strain jl.121 |
| Strain 4 | Strain jl.21 |
| Strain 5 | Strain jl.26 |
| Strain 6 | Strain jl.27 |
| Strain 7 | Bacillus mediterraneensis jl.44 |
| Strain 8 | Strain jl.68 |
| Strain 9 | Strain jl.83 |
| Strain 10 | Strain jl.116 |
| Strain 11 | Strain jl.119 |
| Strain 12 | Strain jl.45 |
| Strain 13 | Strain jl.77 |

In some cases, a bacterial strain comprises Strain jl.83, Strain jl.27, Strain jl.77, Strain jl.68, Strain jl.121, or Strain jl.21. In some instances, the bacterial strains herein are identified by a 16s rRNA sequence. In some cases, Strain jl.83 comprises the 16s rRNA sequence of SEQ ID NO: 4. In some cases, Strain jl.83 comprises a 16s rRNA sequence with at least: 95%, 96%, 97%, 98%, or 99% sequence identity to SEQ ID NO: 4. In some cases, Strain jl.27 comprises the 16s rRNA sequence of SEQ ID NO: 1. In some cases, Strain jl.27 comprises a 16s rRNA sequence with at least: 95%, 96%, 97%, 98%, or 99% sequence identity to SEQ ID NO: 1. In some cases, Strain jl.77 comprises the 16s rRNA sequence of SEQ ID NO: 2. In some cases, Strain jl.77 comprises a 16s rRNA sequence with at least: 95%, 96%, 97%, 98%, or 99% sequence identity to SEQ ID NO: 2. In some cases, Strain jl.68 comprises the 16s rRNA sequence of SEQ ID NO: 5. In some cases, Strain jl.68 comprises a 16s rRNA sequence with at least: 95%, 96%, 97%, 98%, or 99% sequence identity to SEQ ID NO: 5. In some cases, Strain jl.121 comprises the 16s rRNA sequence of SEQ ID NO: 6. In some cases, Strain jl.121 comprises a 16s rRNA sequence with at least: 95%, 96%, 97%, 98%, or 99% sequence identity to SEQ ID NO: 6. In some cases, Strain jl.21 comprises the 16s rRNA sequence of SEQ ID NO: 3. In some cases, Strain jl.21 comprises a 16s rRNA sequence with at least: 95%, 96%, 97%, 98%, or 99% sequence identity to SEQ ID NO: 3.

In some cases, a bacterial strain comprises Strain jl.19, Strain jl.39, Strain jl.26, Strain jl.116, Strain jl.119, or Strain jl.45. In some cases, Strain jl.19 comprises the 16s rRNA sequence of SEQ ID NO: 16. In some cases, Strain jl.19 comprises a 16s rRNA sequence with at least: 95%, 96%, 97%, 98%, or 99% sequence identity to SEQ ID NO: 16. In some cases, Strain jl.39 comprises the 16s rRNA sequence of SEQ ID NO: 17. In some cases, Strain jl.39 comprises a 16s rRNA sequence with at least: 95%, 96%, 97%, 98%, or 99% sequence identity to SEQ ID NO: 17. In some cases, Strain jl.26 comprises the 16s rRNA sequence of SEQ ID NO: 18. In some cases, Strain jl.26 comprises a 16s rRNA sequence with at least: 95%, 96%, 97%, 98%, or 99% sequence identity to SEQ ID NO: 18. In some cases, Strain jl.116 comprises the 16s rRNA sequence of SEQ ID NO: 19. In some cases, Strain jl.116 comprises a 16s rRNA sequence with at least: 95%, 96%, 97%, 98%, or 99% sequence identity to SEQ ID NO: 19. In some cases, Strain jl.119 comprises the 16s rRNA sequence of SEQ ID NO: 20. In some cases, Strain jl.119 comprises a 16s rRNA sequence with at least: 95%, 96%, 97%, 98%, or 99% sequence identity to SEQ ID NO: 20. In some cases, Strain jl.45 comprises the 16s rRNA sequence of SEQ ID NO: 21. In some cases, Strain jl.45 comprises a 16s rRNA sequence with at least: 95%, 96%, 97%, 98%, or 99% sequence identity to SEQ ID NO: 21. In some cases, a bacterial strain herein comprises a 16s rRNA sequence with at least: 95%, 96%, 97%, 98%, or 99% sequence identity over at least 500 bases, 600 bases, 700 bases, 800 bases, 900 bases, 1000 bases, or 1100 bases to any one of SEQ ID NOS: 1-6 or 16-21. In some cases, a bacterial strain herein comprises a 16s rRNA sequence with 100% sequence identity over at least 500 bases, 600 bases, 700 bases, 800 bases, 900 bases, 1000 bases, or 1100 bases to any one of SEQ ID NOS: 1-6 or 16-21.

In some instances, the bacterial strains herein are identified by an identifier sequence. For example, an identifier sequence is a sequence found in the genome and/or accessory genome such as a plasmid that is used to identify a bacterial strain. In some cases, Strain jl.83 comprises the identifier sequence of SEQ ID NO: 11, SEQ ID NO: 12, or both. In some cases, Strain jl.83 comprises an identifier sequence with at least: 90%, 91%, 92%, 93%, 94%, 95%, 96%, 97%, 98%, or 99% sequence identity to SEQ ID NO: 11. In some cases, Strain jl.83 comprises an identifier sequence with at least: 90%, 91%, 92%, 93%, 94%, 95%, 96%, 97%, 98%, or 99% sequence identity to SEQ ID NO: 12. In some cases, Strain jl.27 comprises the identifier sequence of SEQ ID NO: 15. In some cases, Strain jl.27 comprises an identifier sequence with at least: 90%, 91%, 92%, 93%, 94%, 95%, 96%, 97%, 98%, or 99% sequence identity to SEQ ID NO: 15. In some cases, Strain jl.77 comprises the identifier sequence of SEQ ID NO: 7, SEQ ID NO: 8, or both. In some cases, Strain jl.77 comprises an identifier sequence with at least: 90%, 91%, 92%, 93%, 94%, 95%, 96%, 97%, 98%, or 99% sequence identity to SEQ ID NO: 7. In some cases, Strain jl.77 comprises an identifier sequence with at least: 90%, 91%, 92%, 93%, 94%, 95%, 96%, 97%, 98%, or 99% sequence identity to SEQ ID NO: 8. In some cases, Strain jl.68 comprises the identifier sequence of SEQ ID NO: 13. In some cases, Strain jl.68 comprises an identifier sequence with at least: 90%, 91%, 92%, 93%, 94%, 95%, 96%, 97%, 98%, or 99% sequence identity to SEQ ID NO: 13. In some cases, Strain jl.121 comprises the identifier sequence of SEQ ID NO: 14. In some cases, Strain jl.121 comprises an identifier sequence with at least: 90%, 91%, 92%, 93%, 94%, 95%, 96%, 97%, 98%, or 99% sequence identity to SEQ ID NO: 14. In some cases, Strain jl.21 comprises the identifier sequence of SEQ ID NO: 9, SEQ ID NO: 10, or both. In some cases, Strain jl.21 comprises an identifier sequence with at least: 90%, 91%, 92%, 93%, 94%, 95%, 96%, 97%, 98%, or 99% sequence identity to SEQ ID NO: 9. In some cases, Strain jl.21 comprises an identifier sequence with at least: 90%, 91%, 92%, 93%, 94%, 95%, 96%, 97%, 98%, or 99% sequence identity to SEQ ID NO: 10. In some cases, a bacterial strain herein comprises an identifier sequence with at least: 95%, 96%, 97%, 98%, or 99% sequence identity over at least 500 bases, 600 bases, 700 bases, 800 bases, 900 bases, 1000 bases, 1100 bases, or 1200 bases to any one of SEQ ID NOS: 7-15. In some cases, a bacterial strain herein comprises an identifier sequence with 100% sequence identity over at least 500 bases, 600 bases, 700 bases, 800 bases, 900 bases, 1000 bases, 1100 bases or 1200 bases to any one of SEQ ID NOS: 7-15.

In some embodiments, provided herein are bacteria comprising a sequence of SEQ ID NO: 11, SEQ ID NO: 12, or both. In some cases, provided herein are bacteria comprising a sequence with at least: 90%, 91%, 92%, 93%, 94%, 95%, 96%, 97%, 98%, or 99% sequence identity to SEQ ID NO: 11. In some cases, provided herein are bacteria comprising a sequence with at least: 90%, 91%, 92%, 93%, 94%, 95%, 96%, 97%, 98%, or 99% sequence identity to SEQ ID NO: 12. In some cases, provided herein are bacteria comprising a sequence of SEQ ID NO: 15. In some cases, provided herein are bacteria comprising a sequence with at least: 90%, 91%, 92%, 93%, 94%, 95%, 96%, 97%, 98%, or 99% sequence identity to SEQ ID NO: 15. In some cases, provided herein are bacteria comprising a sequence of SEQ ID NO: 7, SEQ ID NO: 8, or both. In some cases, provided herein are bacteria comprising a sequence with at least: 90%, 91%, 92%, 93%, 94%, 95%, 96%, 97%, 98%, or 99% sequence identity to SEQ ID NO: 7. In some cases, provided herein are bacteria comprising a sequence with at least: 90%, 91%, 92%, 93%, 94%, 95%, 96%, 97%, 98%, or 99% sequence identity to SEQ ID NO: 8. In some cases, provided herein are bacteria comprising a sequence of SEQ ID NO: 13. In some cases, provided herein are bacteria comprising a sequence with at least: 90%, 91%, 92%, 93%, 94%, 95%, 96%, 97%, 98%, or 99% sequence identity to SEQ ID NO: 13. In some cases, provided herein are bacteria comprising a sequence of SEQ ID NO: 14. In some cases, provided herein are bacteria comprising a sequence with at least: 90%, 91%, 92%, 93%, 94%, 95%, 96%, 97%, 98%, or 99% sequence identity to SEQ ID NO: 14. In some cases provided herein are bacteria comprising a sequence of SEQ ID NO: 9, SEQ ID NO: 10, or both. In some cases, provided herein are bacteria comprising a sequence with at least: 90%, 91%, 92%, 93%, 94%, 95%, 96%, 97%, 98%, or 99% sequence identity to SEQ ID NO: 9. In some cases, provided herein are bacteria comprising a sequence with at least: 90%, 91%, 92%, 93%, 94%, 95%, 96%, 97%, 98%, or 99% sequence identity to SEQ ID NO: 10.

In some embodiments, a composition herein can comprise cell free components, such as supernatant from any one of the strains listed in Table 2. In some cases, a composition herein can comprise cell free components, from any combination of strains listed in Table 2. In some cases, a composition herein can comprise cell free components from one or more bacterial strains with at least 95%, 96%, 97%, 98%, or 99% sequence identity to any one of SEQ ID NOS: 1-6 or 16-21. In some cases, a composition herein can comprise cell free components from one or more bacterial strains comprising any one of SEQ ID NOS: 1-6 or 16-21. In some cases, a composition herein can comprise cell free components from a bacterial strain with at least 95%, 96%, 97%, 98%, or 99% sequence identity to SEQ ID NO: 5. In some cases, a composition herein can comprise cell free components from a bacterial strain comprising SEQ ID NO: 5.

In some cases, a composition described herein comprises one or more bacterial strains and one or more bacterial species. For example, a composition described herein comprises two bacterial species and one bacterial strain.

In some embodiments, a composition comprises two or more bacterial species or strains. In some embodiments, a composition comprises at least two of the following bacterial species: Staphylococcus cohnii, Staphylococcus capitis, Staphylococcus caprae, Bacillus tropicus, Bacillus mycoides, Bacillus wiedmannii, Bacillus mediterraneensis, Bacillus amyloliquefaciens, Bacillus velezensis, Bacillus cereus, Bacillus cecembensis, Kocuria marina, and Lysinibacillus macroides. In some embodiments, a composition comprises at least two of the following bacterial species: Bacillus cereus, Staphylococcus epidermidis, Bacillus nakamurai, Bacillus vallismortis, Lysinibacillus boronitolerans, Bacillus paranthracis, Bacillus thuringiensis, Psychrobacillus sp. INOP01, Lysinibacillus sp. FN11, Bacillus sp. Lzh-5, or Staphylococcus capitis. In some embodiments, a composition comprises at least two of the following bacterial species: Bacillus velezensis, Bacillus wiedmannii, Lysinibacillus macrolides, Bacillus amyloliquefaciens, Staphylococcus caprae, and Bacillus tropicus. In some embodiments, a composition comprises at least two of the following bacterial strains: Strain jl.83, Strain jl.27, Strain jl.77, Strain jl.68, Strain jl.121, and Strain jl.21. In some cases, a composition comprises bacterial strains Strain jl.83, and Strain jl.27. In some cases, a composition comprises bacterial strains Strain jl.68, and Strain jl.27. In some cases, a composition comprises bacterial strains Strain jl.68, and Strain jl.21. In some cases, a composition comprises bacterial strains Strain jl.83, and Strain jl.77. In some cases, a composition comprises bacterial strains Strain jl.27 and Strain jl.121. In some cases, a composition comprises bacterial strains Strain jl.21 and Strain jl.121.

In some embodiments, a composition comprises three or more bacterial species or strains. In some embodiments, a composition comprises at least three of the following bacterial species: Staphylococcus cohnii, Staphylococcus capitis, Staphylococcus caprae, Bacillus tropicus, Bacillus mycoides, Bacillus wiedmannii, Bacillus mediterraneensis, Bacillus amyloliquefaciens, Bacillus velezensis, Bacillus cereus, Bacillus cecembensis, Kocuria marina, and Lysinibacillus macroides. In some embodiments, a composition comprises at least three of the following bacterial species: Bacillus velezensis, Bacillus wiedmannii, Lysinibacillus macrolides, Bacillus amyloliquefaciens, Staphylococcus caprae, and Bacillus tropicus. In some embodiments, a composition comprises at least three of the following bacterial strains: Strain jl.83, Strain jl.27, Strain jl.77, Strain jl.68, Strain jl.121, and Strain jl.21.

In some embodiments, a composition described herein comprises three bacterial species or strains. In some embodiments, a composition comprises a bacterial species Bacillus velezensis, Bacillus wiedmannii, and Lysinibacillus macrolides. In some cases, a composition comprises bacterial strains Strain jl.83, Strain jl.27, and Strain jl.77. In some embodiments, a composition comprises a bacterial species Bacillus amyloliquefaciens, Bacillus wiedmannii, and Staphylococcus caprae. In some cases, a composition comprises bacterial strains Strain jl.68, Strain jl.27, and Strain jl.121. In some embodiments, a composition comprises a bacterial species Bacillus amyloliquefaciens, Bacillus tropicus, and Staphylococcus caprae. In some cases, a composition comprises bacterial strains Strain jl.68, Strain jl.21, and Strain jl.121. In some embodiments, a composition comprises a bacterial species combination from Table 3. In some embodiments, a composition is a bacterial species combination from Table 3. In some embodiments, a composition comprises a bacterial strain combination from Table 4. In some embodiments, a composition is a bacterial strain combination from Table 4.

TABLE 3
Three bacterial species combinations

| Combination | |
|---|---|
| Number | Bacterial species names |
| Combination 1 | Bacillus amyloliquefaciens |
| | Staphylococcus cohnii |
| | Bacillus cereus |
| Combination 2 | Bacillus amyloliquefaciens |
| | Bacillus tropicus |
| | Staphylococcus caprae |
| Combination 3 | Bacillus amyloliquefaciens |
| | Bacillus wiedmannii |
| | Bacillus cecembensis |
| Combination 4 | Bacillus velezensis |
| | Bacillus mycoides |
| | Kocuria marina |
| Combination 5 | Bacillus velezensis |
| | Bacillus mycoides |
| | Lysinibacillus macroides |
| Combination 6 | Bacillus velezensis |
| | Bacillus wiedmannii |
| | Bacillus mediterraneensis |
| Combination 7 | Bacillus amyloliquefaciens |
| | Bacillus wiedmannii |
| | Staphylococcus caprae |
| Combination 8 | Bacillus amyloliquefaciens |
| | Staphylococcus capitis |
| | Bacillus cecembensis |
| Combination 9 | Bacillus velezensis |
| | Staphylococcus capitis |
| | Bacillus mediterraneensis |
| Combination 10 | Bacillus amyloliquefaciens |
| | Staphylococcus capitis |
| | Staphylococcus caprae |
| Combination 11 | Bacillus velezensis |
| | Bacillus wiedmannii |
| | Lysinibacillus macroides |

TABLE 4
Three bacterial strain combinations

| Combination | |
|---|---|
| Number | Bacterial strain names |
| Combination 1 | Strain jl.68 |
| | Strain jl.19 |
| | Strain jl.116 |
| Combination 2 | Strain jl.68 |
| | Strain jl.21 |
| | Strain jl.121 |
| Combination 3 | Strain jl.68 |
| | Strain jl.27 |
| | Strain jl.119 |
| Combination 4 | Strain jl.83 |
| | Strain jl.26 |
| | Strain jl.45 |
| Combination 5 | Strain jl.83 |
| | Strain jl.26 |
| | Strain jl.77 |
| Combination 6 | Strain jl.83 |
| | Strain jl.27 |
| | Bacillus mediterraneensis jl.44 |
| Combination 7 | Strain jl.68 |
| | Strain jl.27 |
| | Strain jl.121 |
| Combination 8 | Strain jl.68 |
| | Strain jl.39 |
| | Strain jl.119 |
| Combination 9 | Strain jl.83 |
| | Strain jl.39 |
| | Bacillus mediterraneensis jl.44 |
| Combination 10 | Strain jl.68 |
| | Strain jl.39 |
| | Strain jl.121 |
| Combination 11 | Strain jl.83 |
| | Strain jl.27 |
| | Strain jl.77 |

In some embodiments, a composition described herein comprises one bacterial species or strain. In some embodiments, a composition comprises a second bacterial species or strain. In some embodiments, a composition comprises Bacillus velezensis. In some cases, a composition comprises Strain jl.83. In some embodiments, a composition comprises Bacillus wiedmannii. In some cases, a composition comprises Strain jl.27. In some embodiments, a composition comprises Lysinibacillus macroides. In some cases, a composition comprises Strain jl.77. In some embodiments, a composition comprises Bacillus tropicus. In some cases, a composition comprises Strain jl.21. In some embodiments, a composition comprises Bacillus amyloliquefaciens. In some cases, a composition comprises Strain jl.68. In some embodiments, a composition comprises Staphylococcus caprae. In some cases, a composition comprises Strain jl.121. In some embodiments, a composition comprises Staphylococcus cohnii. In some cases, a composition comprises Strain jl.19. In some embodiments, a composition comprises Staphylococcus capitis. In some cases, a composition comprises Strain jl.39. In some embodiments, a composition comprises Bacillus mycoides. In some cases, a composition comprises Strain jl.26. In some embodiments, a composition comprises Bacillus mediterraneensis. In some cases, a composition comprises Bacillus mediterraneensis jl.44. In some embodiments, a composition comprises Bacillus cereus. In some cases, a composition comprises Strain jl.116. In some embodiments, a composition comprises Bacillus cecembensis. In some cases, a composition comprises Strain jl.119. In some embodiments, a composition comprises Kocuria marina. In some cases, a composition comprises Strain jl.45.

In some embodiments, Bacillus velezensis is Bacillus velezensis DSM 23117, which can be obtained from the DSMZ-German Collection of Microorganisms and Cell Cultures depository. In some embodiments, Bacillus wiedmannii is Bacillus wiedmannii DSM 102050, which can be obtained from the DSMZ-German Collection of Microorganisms and Cell Cultures depository. In some embodiments, Lysinibacillus macroides is Lysinibacillus macroides DSM 54, which can be obtained from the DSMZ-German Collection of Microorganisms and Cell Cultures depository. In some embodiments, Bacillus amyloliquefaciens is Bacillus amyloliquefaciens DSM 7, which can be obtained from the DSMZ-German Collection of Microorganisms and Cell Cultures depository. In some embodiments, Staphylococcus caprae is Staphylococcus caprae DSM 20608, which can be obtained from the DSMZ-German Collection of Microorganisms and Cell Cultures depository. In some embodiments, Bacillus amyloliquefaciens is Bacillus amyloliquefaciens ATCC 23350 (Bacillus amyloliquefaciens FIFO 15535) which can be obtained from the ATCC (American Type Culture Collection) depository. In some embodiments, Bacillus tropicus is Bacillus tropicus ATCC 4342 (Bacillus tropicus NRS 731), which can be obtained from the ATCC (American Type Culture Collection) depository.

In some embodiments, when administered to a subject, a bacterial species or a strain described herein can reduce or eliminate colonization of the skin of pathogenic: bacteria, fungi or viruses. In some cases, a composition described herein is used to kill a pathogenic bacteria, fungi or virus. In some cases, a composition described herein is used to suppress growth of a pathogenic bacteria, fungi or virus. In some cases, a composition described herein is used to control growth of a pathogenic bacteria, fungi or virus. In some cases, a composition described herein is used to control virulence of a pathogenic bacteria, fungi or virus. In some cases, a composition comprising one or more bacterial species or strains when administered can reduce or eliminate colonization of the skin of pathogenic: bacteria, fungi or viruses. Skin pathogenic bacteria include, without limitation, Staphylococcus sp., Micrococcus sp., Corynebacterium sp., Staphylococcus aureus, Streptococcus pyogenes, Pseudomonas aeruginosa, Pasteurella multocida, Capnocytophaga canimorsus, Bartonella sp., Klebsiella rhinoscleromatis, and Vibrio vulnificus. In some cases, a pathogenic bacteria comprises Gardnerella vaginalis. Pathogenic skin fungi include, without limitation, ascomycete dermatophytes, including the genera Aspergillus, Trichophyton, Microsporum, and Epidermophyton, and basidiomycete fungi in the genus Malassezia. In some cases, a skin pathogenic fungi comprises Malassezia pachydermatis, Candida albicans, Microsporum canis, and Trichophyton mentagrophytes. Exemplary skin pathogenic viruses include, without limitation, Herpes sp., Herpes zoster., Herpes simplex., Molluscum contagiosum, and Human papillomavirus. Such reduction of pathogenic bacteria, fungi or viruses may be in any location of the skin, for example the forearm, the leg, or the stomach of a subject. In some cases, a composition herein can reduce S. aureus toxin production, reduce fungal (e.g., Candida) hyphal formation, reduce Malassezia sebum metabolism, or reduce Gardnerella biofilm formation.

Provided herein are compositions comprising a bacterial species and/or a bacterial strain described herein in an amount sufficient for modulations of the expression of a gene in a pathogenic microorganism, or a modulation in the expression of a protein in a pathogenic microorganism. In some cases, the modulation is an increase of the gene expression or the protein expression. In some cases, the modulation is a decrease of the gene expression or the protein expression. In some cases, the composition provides for a reduction in the expression of a gene or protein from S. aureus or S. pyogenes. Exemplary genes for modulation and suppression include, without limitation, a virulence gene, a metabolism gene, a transcription gene, a translation gene, a protein processing gene, a protein folding gene, a secretion gene, a cell division gene, a biosynthesis gene, a cell wall gene, a cell membrane gene, an antibiotic resistance gene, and a protein coded by one of these genes. In some cases, the gene or protein is associated with growth, regulation (e.g., virulence regulation, metabolic regulation, or growth regulation), direct virulence such as a toxin, stress, or a metabolic state. In some cases, the gene is gmk, agr, psmA, sigB, saeR, and/or ccpA. In some cases, the protein is encoded by gmk, agr, psmA, sigB, saeR, and/or ccpA. In some cases, the expression of a gene is measured by a promoter-reporter strain. For example, a promoter of a gene of interest such as, gmk, agr, psmA, sigB, saeR, or ccpA can be fused to a fluorescent reporter such as GFP, or luciferase to measure the amount of gene expression. Exemplary genes for promoter-reporter S. aureus strains are shown in Table 5. In some cases, gene expression is used to assess constitutive metabolic function, quorum sensing, a toxin that damages host tissue, a stress response sigma factor, virulence regulation, and/or carbon catabolite repression. In some cases, gene expression is measured by a Northern blot, a Western blot, a promoter-reporter gene, a microarray, a PCR assay such as a reverse transcription polymerase chain reaction, a Serial Analysis of Gene Expression (SAGE), a next generation sequencing technique such as RNA Seq, or a combination of these methods. In some cases, the expression of a protein is measured. In some cases, protein expression is measured by a Western blot, an enzyme-linked immunoassay (ELISA), an SDS-PAGE gel, a Bicinchoninic acid (BCA) assay, or any assay that quantifies protein expression and/or amounts.

TABLE 5
Promoter-Reporter Stains

| | Protein | Associated | Protein Genbank | Genome Genbank |
|---|---|---|---|---|
| Promoter | Function | with | Reference for gene | Reference |
| gmk | Constitutive | Growth | WP_000368227.1 | GenBank: |
| | metabolic | | | CP000253.1 Range: |
| | function | | | 1126551 to 1126850 |
| agr | Quorum | Virulence | ADF28639.1 | GenBank ID: |
| | sensing | (regulatory) | | CP000253.1 Range: |
| | induction | | | 2093636 to 2093876 |
| psmA | Toxin that | Virulence | WP_014373781.1 | GenBank ID: |
| | damages | (direct) | | CP000253.1 Range: |
| | host tissue | | | 412992 to 413258 |
| sigB | Stress | Stress | CAC6986262.1 | GenBank ID: |
| | response | (regulatory) | | CP000253.1 Range: |
| | sigma factor | | | 2133482 to 2133781 |
| saeR | Broad | Virulence | WP_000149344.1 | GenBank ID: |
| | virulence | (regulatory) | | CP000253.1 Range: |
| | regulation | | | 700936 to 701683 |
| ccpA | Carbon | Metabolic | WP_000219066.1 | GenBank ID: |
| | catabolite | state | | CP000253.1 Range: |
| | repression | | | 1757336 to 1757835 |

In some embodiments, a composition comprising a bacterial species and/or a bacterial strain can further comprise one or more compounds. In some cases, a compound can be an environmental component. In some cases, a compound comprises a carbon source, a nitrogen source, a phosphorus source, a sulfur source, or a metal ion source. In some cases, a compound can produce a synergistic effect with a bacterial composition. For example, a compound can enhance the inhibition of S. aureus expression. In some cases, a compound increases the potency of a bacterial composition. In some cases, a compound comprises thiamine (vitamin B-1) or a salt thereof. In some cases, a compound comprises acetate, beta-alanine, bicarbonate, biotin, butyrate, caffeine, citrate, creatine, D-cellobiose, D-fructose, D-glucosamine, D-glucose, D-mannitol, D-raffinose, D-sorbitol, D-sucrose, D-trehalose, D-xylose, formate, GlcNAc, glycerol, glycine, L-alanine, L-arabinose, L-arginine, L-citrulline, L-glutamine, L-hydroxyproline, L-isoleucine, L-leucine, L-methionine, L-ornithine, L-proline, L-serine, L-taurine, L-threonine, L-valine, L-ascorbate, L-lactate, nicotinamine, polysorbate 20, polysorbate 80, propionate, pyruvate, succinate, thiamine, triethanolamine, or urea.

### Routes of Administration, Excipients, Dosing, and Dosage Forms

In some embodiments, the terms “administer,” “administering”, “administration,” and the like, as used herein, can refer to methods that can be used to enable delivery of compositions described herein, to the desired site of biological action. In some cases, delivery comprises topical administration. In some cases, delivery can include injection, inhalation, catheterization, gastrostomy tube administration, intravenous administration, intraosseous administration, ocular administration, otic administration, topical administration, transdermal administration, oral administration, rectal administration, nasal administration, intravaginal administration, intracavernous administration, intracerebral administration, transurethral administration, buccal administration, sublingual administration, or a combination thereof. Delivery can include direct application to the affect tissue or region of the body. Delivery can include a parenchymal injection, an intra-thecal injection, an intra-ventricular injection, or an intra-cisternal injection. A composition provided herein can be administered by any method. A method of administration can be by intraarterial injection, intracerebroventricular injection, intracisternal injection, intramuscular injection, intraorbital injection, intraparenchymal injection, intraperitoneal injection, intraspinal injection, intrathecal injection, intravenous injection, intraventricular injection, stereotactic injection, subcutaneous injection, epidural, or any combination thereof. Delivery comprises parenteral administration (e.g., intravenous, subcutaneous, intrathecal, intraperitoneal, intramuscular, intravascular or infusion administration). In some embodiments, delivery comprises a nanoparticle, a microparticle, a viral-like particle, a liposome, an exosome, an extracellular vesicle, a microneedle, an implant, or a combination thereof. In some cases, delivery is from a device. In some cases, delivery is by an enema, an eye drop, a nasal spray, a spray, an ear drop, or any combination thereof. In some cases, delivery is in the form of a solutions, a suspension, an emulsions, a tablet, a pill, a pellet, a capsule, a capsule including a liquid, a powder, a sustained-release formulation, a directed release formulation, lyophylates (freeze dried/lyophilized), an aerosols, a spray, a granules, a powder, or a syrup. In some cases, delivery comprises an inhaler, a diffuser, a nebulizer, or a combination thereof. Delivery can include topical administration (such as a lotion, a cream, a patch, a film, a gel, a spray, a drip, a liquid formulation, an ointment, a suspension, an emulsion, a tincture, a foam, a powder, a paste, or an oil) to an external surface of a surface, such as a skin. In some instances, a subject can administer the composition in the absence of supervision. In some instances, a subject can administer the composition under the supervision of a medical professional (e.g., a physician, nurse, physician's assistant, orderly, hospice worker, etc.). In some cases, a medical professional can administer the composition. In some cases, the subject can administer the composition.

To facilitate administration, pharmaceutical compositions described herein may include one or more pharmaceutically acceptable excipients. Example pharmaceutically acceptable excipients include, without limitation, diluents, adjuvants, excipients, water, oils (including petroleum, animal, vegetable, natural oils, or synthetic oils.). Further examples include saline, gum acacia, gelatin, starch paste, talc, keratin, colloidal silica, and urea. Such excipients may include binders such as ethyl cellulose, carboxymethylcellulose, microcrystalline cellulose, or gelatin; excipients such as starch, lactose or dextrins; disintegrating agents such as alginic acid, sodium alginate, Primogel, and cornstarch; lubricants such as magnesium stearate or Sterotex; glidants such as colloidal silicon dioxide; sweetening agents such as sucrose or saccharin, a flavoring agent such as peppermint, methyl salicylate or orange flavoring, or coloring agents. Further examples of excipients include polyethylene glycol, cyclodextrin, oils, or any other similar liquid carrier that may be formulated into a capsule. Still further examples of excipients include sterile diluents such as water, saline solution, physiological saline, Ringer's solution, isotonic sodium chloride, phosphate-buffered saline, fixed oils such as synthetic mono or digylcerides, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose, thickening agents, lubricating agents, and coloring agents. In some embodiments of the invention, the pharmaceutically acceptable carrier comprises a growth medium that can support the growth and/or static existence of beneficial bacteria described herein in the context of the pharmaceutical composition prior to administration of the pharmaceutical composition to the subject.

In some embodiments, a composition described herein comprises a lyoprotectant. In some cases, a composition described herein comprises a cryoprotectant. In some embodiments, the composition can further comprise an emollient. In some cases, a composition described herein is freeze dried or lyophilized. In some cases, a lyoprotectant can comprise a milk, a sugar, sucrose, lactose, glucose, trehalose, glycerol, mannitol, sorbitol, glycine, alanine, lysine, polyethylene glycol, a dextran or polyvinylpyrrolidone (PVP).

Various other additives can be included in the compositions.

Non-limiting examples of additives include antioxidants, astringents, perfumes, preservatives, emollients, pigments, dyes, humectants, propellants, and sunscreen agents, as well as other classes of materials whose presence may be desirable pharmaceutically or otherwise. In some cases, an emollient can comprise a moisturizer.

Non-limiting examples of optional additives include preservatives, such as sorbate; solvents, such as isopropanol and propylene glycol; astringents, such as menthol and ethanol; emollients, such as polyalkylene methyl glucosides; humectants, such as glycerin; emulsifiers, such as glycerol stearate, PEG-100 stearate, polyglyceryl-3 hydroxyluryl ether, and polysorbate 60; sorbitol and other polyhydroxy alcohols, such as polyethylene glycol; sunscreen agents, such as methoxy octyl cinnamate (Parsol MCX) and butyl methoxy benzoylmethane (Parsol 1789); antioxidants, such as ascorbic acid (vitamin C), α-tocopherol (Vitamin E), β-tocopherol, γ-tocopherol, δ-tocopherol, ε-tocopherol, ζ1-tocopherol, ζ2-tocopherol, η-tocopherol, and retinol (vitamin THE); essential oils, ceramides, essential fatty acids, mineral oils, vegetable oils (eg soy oil, palm oil, shea butter liquid fraction, sunflower oil), animal oils (eg perhydrosqualene), synthetic oils, silicone oils or waxes (for example, cyclomethicone and dimethicone), fluorinated oil (usually perfluoropolyethers), fatty alcohols (for example, cetyl alcohol), and waxes (for example, beeswax, carnauba wax and paraffin wax); modifiers of skin feel; and thickeners and structuring agents, such as swellable clays and cross-linked carboxypolialkylenes.

In some cases, a composition comprises a therapeutic agent. In some instances, a therapeutic agent is a drug or a compound. In some cases, a therapeutic agent can comprise a salt of a therapeutic agent. As used herein, a therapeutic agent, can also refer to the free-base, acid, salts, esters, and mixtures of a therapeutic agent. In some cases, a salt can comprise a pharmaceutically acceptable salt. In some cases, a salt comprises an HCl salt, an ascorbic acid salt, a mandelic acid salt, an aspartic acid salt, a carbonic acid salt, a citric acid salt, a formic acid salt, a glutamic acid salt, a lactic acid salt, a lauric acid salt, a maleic acid salt, a palmitic acid salt, or a phosphoric acid salt.

In some embodiments, administering comprises administering one or more additional therapeutics. In some cases, a second therapeutic is administered. In some instances, a second therapy is administered concurrently or consecutively with a first therapy. In some instances, a second therapy is administered in parallel with the first therapy. In some instances, a second therapy can enhance the efficacy of a first therapy. In some instances, a second therapy is comprised in a single dose with a first therapy. For example, an additional therapeutic can be added to a bacterial mixture described herein. In some instances, a second therapy may be comprised in a separate dose from the first therapy. In some cases, a second therapy comprises a cream, wet dressing, a light therapy (e.g., phototherapy), or behavior modification. In some cases, a second therapy comprises an antibody, such as a human monoclonal antibody. In some cases, a second therapy comprises an antibiotic. In some cases, a second therapy comprises a corticosteroid, a calcineurin inhibitor, pimecrolimus, tacrolimus, crisaborole, doxepin, narrowband ultraviolet B (NBUVB) phototherapy, ultraviolet A1 (UVA1) phototherapy, an antihistamine, diphenhydramine, hydroxyzine, cyproheptadine, fexofenadine, cetirizine, loratadine, cyclosporine, dupilumab, tralokinumab, nemolizumab, an anti-OX40 antibody, a JAK1/JAK2 inhibitor, a PDE4 inhibitor, upadacitinib, abrocitinib, azathioprine, an emollient, a moisturizer, an interleukin inhibitor, methotrexate, mycophenolate mofetil, or interferon gamma. In some cases, a second therapy comprises a ephalosporin (e.g., cefazolin, cephalothin and cephalexin), clindamycin, lincomycin, erythromycin, flucloxacillin, dicloxacillin, rifampicin, a lincosamide (e.g., clindamycin, lincomycin), cotrimoxazole, linezolid, quinupristin, dalfopristin, trimethoprim, sulfamethoxazole, fusidic acid, penicillin, methicillin, vancomycin, or a salt of any of these. In some instances, a second therapeutic or a first therapy can be administered buccally, enterally, by inhalation administration, by infusion administration, intramuscularly, intrathecally, intravenously, nasally, ophthalmically, orally, otically, by rectal administration, subcutaneously, sublingually, topically, transdermally or by any administration method.

Dosing may include single or multiple administrations of pharmaceutical compositions described herein. Examples include: multiple times a day, daily, every other day, 1, 2, 3, 5, 6, or 7 times a week, weekly, or less often, a single administration, a course of treatment involving several treatments on a regular or irregular basis, or multiple administrations for a period of time a disease or condition is treated. In some cases, dosing can occur every day, 2 days, 3 days, 4 days, 5 days, 6 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 1 year, 2 years, or as needed. The dosing regimen, including the regularity of and mode of administration, may be dependent on factors including but not limited to the subject being treated; the severity of the condition; the manner of administration, the stage of colonization, the amount of a pathogenic organism present, the presence of one or more other conditions such as pregnancy, infancy, or the presence of one or more additional diseases. In some cases, the subject is an infant. The infant can be up to 6 months old, up to 12 months old, or up to 24 months old. In some cases, the subject is a child. The child may be 2 years to 21 years old. In some cases, the child may be up to: 5, 7, 12, 18 or 21 years old. In some cases, the subject is an adult. Adults may be 21 years old or more. In some embodiments, the adult is of advanced age, such as 65 years or older.

Administration or application of a composition disclosed herein can be performed for a treatment duration of at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years, at least 3 years, at least 4 years, at about 5 years, at least 6 years, at least 7 years, at least 8 years, at least 9 years, at least 10 years, at least 15 years, at least 20 years, or for life. Administration can be performed repeatedly over a lifetime of a subject, such as once a month or once a year for the lifetime of a subject. Administration can be performed repeatedly over a substantial portion of a subject's life, such as once a month or once a year for at least about 1 year, 5 years, 10 years, 15 years, 20 years, 25 years, 30 years, or more.

Compositions, including pharmaceutical compositions, described herein may comprise a single (unit) dose of bacteria. Compositions described herein may comprise about l0² to about 10¹⁵ colony forming units (cfu) of bacteria, a bacterial species, or a bacterial strain described herein. Compositions described herein may comprise about: 10² to 10¹² cfu, 10³ to 10¹² cfu, 10³ to 10¹¹ cfu, 10³ to 10¹⁰ cfu, 10³ to 10⁹ cfu, 10³ to 10⁸ cfu, 10³ to 10⁷ cfu, 10³ to 10⁶ cfu, 10³ to about 10⁵ cfu, 10³ to 10⁴ cfu, 10⁴ to 10¹² cfu, 10⁴ to 10¹¹ cfu, 10⁴ to 10¹⁰ cfu, 10⁴ to 10⁹ cfu, 10⁴ to 10⁸ cfu, 10⁴ to 10⁷ cfu, 10⁴ to 10⁶ cfu, 10⁵ to 10¹² cfu, 10⁵ to 10¹¹ cfu, about 10⁵ to about 10¹⁰ cfu, 10⁶ to 10¹² cfu, 10⁷ to 10¹² cfu, 10⁸ to 10¹² cfu, 10⁹ to 10¹² cfu, 10¹⁰ to 10¹² cfu, 10¹¹ to 10¹² cfu, or 10⁶ to 10¹⁰ cfu of bacteria, a bacterial species, or a bacterial strain described herein. In some embodiments, compositions comprise about 10³ cfu, about 10⁴ cfu, about 10⁵ cfu, about 10⁶ cfu, about 10⁷ cfu, about 10⁸ cfu, about 10⁹ cfu, about 10¹⁰ cfu, about 10¹¹ cfu, about 10¹² cfu, or about 10¹³ cfu of bacteria, a bacterial species, or a bacterial strain described herein.

Compositions, such as pharmaceutical compositions, described herein may comprise 10² to 10¹⁵ colony forming units (cfu) of bacteria, a bacterial species, or a bacterial strain described herein per mL. Compositions described herein may comprise about 10² to 10¹² cfu, 10³ to 10¹² cfu, 10³ to 10¹¹ cfu, 10³ to 10¹⁰ cfu, 10³ to 10⁹ cfu, 10³ to 10⁸ cfu, 10³ to 10⁷ cfu, 10³ to 10⁶ cfu, 10³ to about 10⁵ cfu, 10³ to 10⁴ cfu, 10⁴ to 10¹² cfu, 10⁴ to 10¹¹ cfu, 10⁴ to 10¹⁰ cfu, 10⁴ to 10⁹ cfu, 10⁴ to 10⁸ cfu, 10⁴ to 10⁷ cfu, 10⁴ to 10⁶ cfu, 10⁵ to 10¹² cfu, 10⁵ to 10¹¹ cfu, 10⁵ to 10¹⁰ cfu, 10⁶ to 10¹² cfu, 10⁷ to 10¹² cfu, 10⁸ to 10¹² cfu, 10⁹ to 10¹² cfu, 10¹⁰ to 10¹² cfu, 10¹¹ to 10¹² cfu, or 10⁶ to 10¹⁰ cfu of bacteria, a bacterial species, or a bacterial strain described herein per mL.

Compositions, such as pharmaceutical compositions, described herein may comprise l0² to 10¹⁵ colony forming units (cfu) of bacteria, a bacterial species, or a bacterial strain described herein per gram. Compositions described herein may comprise about 10² to 10¹² cfu, 10³ to 10¹² cfu, 10³ to 10¹¹ cfu, 10³ to 10¹⁰ cfu, 10³ to 10⁹ cfu, 10³ to 10⁸ cfu, 10³ to 10⁷ cfu, 10³ to 10⁶ cfu, 10³ to about 10⁵ cfu, 10³ to 10⁴ cfu, 10⁴ to 10¹² cfu, 10⁴ to 10¹¹ cfu, 10⁴ to 10¹⁰ cfu, 10⁴ to 10⁹ cfu, 10⁴ to 10⁸ cfu, 10⁴ to 10⁷ cfu, 10⁴ to 10⁶ cfu, 10⁵ to 10¹² cfu, 10⁵ to 10¹¹ cfu, 10⁵ to 10¹⁰ cfu, 10⁶ to 10¹² cfu, 10⁷ to 10¹² cfu, 10⁸ to 10¹² cfu, 10⁹ to 10¹² cfu, 10¹⁰ to 10¹² cfu, 10¹¹ to 10¹² cfu, or 10⁶ to 10¹⁰ cfu of bacteria, a bacterial species, or a bacterial strain described herein per gram.

Compositions described herein may comprise may at least about 0.01% by weight, at least about 0.05% by weight, at least about 0.1% by weight, at least about 0.2% by weight, at least about 0.3% by weight, at least about 0.4% by weight, at least about 0.5% by weight, at least about 0.6% by weight, at least about 0.7% by weight, at least about 0.8% by weight, at least about 0.9% by weight, at least about 1.0% by weight, at least about 1.5% by weight, at least about 2.0% by weight, at least about 3.0% by weight, at least about 4.0% by weight, at least about 5.0% by weight, at least about 6.0% by weight, at least about 7.0% by weight, at least about 8.0% by weight, at least about 9.0% by weight, at least about 10.0% by weight, at least about 11.0% by weight, at least about 12.0% by weight, at least about 13.0% by weight, at least about 14.0% by weight, at least about 15.0% by weight, at least about 16.0% by weight, at least about 17.0% by weight, at least about 18.0% by weight, at least about 19.0% by weight, at least about 20.0% by weight, at least about 25.0% by weight, at least about 30.0% by weight, at least about 35.0% by weight, at least about 40.0% by weight, at least about 45.0% by weight, or at least about 50.0% by weight of bacteria, a bacterial species, or a bacterial strain described herein. In some embodiments, compositions can include from 0.01% to 30% by weight, from about 0.01% to 20% by weight, from 0.01% to 5% by weight, from 0.1% to 30% by weight, from 0.1% to 20% by weight, from 0.1% to about 15% by weight, from 0.1% to 10% by weight, from 0.1% to 5% by weight, from 0.2% to 5% by weight, from 0.3% to 5% by weight, from 0.4% to 5% by weight, from 0.5% to 5% by weight, or from 1% to 5% by weight of bacteria, a bacterial species, or a bacterial strain described herein.

Compositions, including pharmaceutical compositions, described herein may comprise a ratio (cfu to cfu) of about: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1:500, 1:600, 1:700, 1:800, 1:900 or about 1:1000 of a species in Table 1 to another species in Table 1, a strain in Table 2 to another strain in Table 2, or a species in Table 1 to a strain in Table 2. Compositions, including pharmaceutical compositions, described herein may comprise a ratio (cfu to cfu) of about: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1:500, 1:600, 1:700, 1:800, 1:900 or about 1:1000 of multiple strains of Bacillus velezensis, Bacillus wiedmannii, Lysinibacillus macrolides, Bacillus amyloliquefaciens, Staphylococcus caprae, and/or Bacillus tropicus.

In some embodiments, a composition herein comprises a formulation. In some cases, a formulation comprises freeze dried or lyophilized bacteria. In some cases, a formulation comprises reconstituted bacteria. In some cases, an individual species or strain is stored in a container such as a vial. In some instances, a formulation herein comprises a mixture of bacteria from 2, 3, 4, 5, or more reconstituted vials.

### Conditions

In some embodiments, provided herein are compositions for the treatment of a condition or disease of the skin. As described in more detail herein, such conditions or diseases are conditions or disease of the epidermis, the dermis or the hypodermis (e.g., subcutaneous tissue). In some cases, a condition or a disease is a condition or a disease of the stratum corneum, the stratum germinativum, the statum spinosum, the stratum basale, the dermal papilla, a sebaceous gland, a hair follicle, a nerve fiber, a arrector pili muscle, the dermal papilla, a sweat gland, or a combination of any of these. The skin can include the skin of any surface of the body. For example, skin from a head, an arm, a leg, a torso, a stomach, a foot, a toe, a finger, a thumb, a forearm, a back, a groin or any region on the body. In some cases, compositions described comprises isolated bacteria present in an amount sufficient for a reduction in incidence of colonization of a pathogenic bacteria, fungi or virus. In some cases, compositions described comprises isolated bacteria present in an amount sufficient for a reduction in the gene expression of a pathogenic bacteria, fungi or virus. In some cases, compositions described comprises isolated bacteria present in an amount sufficient for a reduction in the metabolism of a pathogenic bacteria, fungi or virus. In some cases, compositions described comprises isolated bacteria present in an amount sufficient for a reduction in the virulence of a pathogenic bacteria, fungi or virus. In some cases, a disease or condition can relate to a bacterial infection. Sources for bacterial infections for treatment with pharmaceutical compositions described herein include, without limitation, S. aureus (methicillin-resistant S. aureus (MRSA) and methicillin-sensitive S. aureus (MSSA)), Gardnerella vaginalis, and Streptococcus pyogenes.

In some embodiments, a skin condition or disease is treated by administering a composition described herein. In some cases, treating can be used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms of an underlying disorder being treated. In some cases, a prophylactic effect can include delaying, preventing, or eliminating the appearance of a disease or condition, delaying or eliminating the onset of symptoms of a disease or condition, slowing, halting, or reversing the progression of a disease or condition. In some cases, an inflammation is treated by administering a composition described herein. Skin conditions and disease for treatment following administration of a composition described herein include, without limitation, a mycoses, a dry skin, an itchy skin, a dermatophytosis (trichophyton), a bacterial folliculitis, a bacterial infection, an inflammatory condition, or a genetic condition. Skin conditions and disease for treatment following administration of a composition described herein comprises a skin and soft tissue infection. In some cases, administration of a composition described herein comprises restoration of an eczema prone microbiome. For example, an eczema prone microbiome can be restored to a healthy or non-eczema prone microbiome. In some instances, a mycoses comprises malassezia folliculitis, tinea versicolor, cutaneous candidiasis, candidal paronychia. In some instances, a dermatophytosis (trichophyton) comprises tinea capitis (scalp ringworm), tinea barbae (beard ringworm), tinea corporis (body ringworm), tinea cruris (“jock itch”), tinea pedis (“athlete's foot”), tinea capitis (scalp ringworm), tinea barbae (beard ringworm), tinea corporis (body ringworm), tinea unguium (nails, onychomycosis), tinea manuum (hand), tinea faciei (face). In some instances, a bacterial infection comprises S. aureus folliculitis, folliculitis, sycosis vulgaris (“barber's itch”), staphylococcal scalded skin syndrome, impetigo, ecthyma, cellulitis, a boil, an abscess, a furuncle, a mastitis, ecthyma, erysipelas, necrotizing fasciitis, secondary skin infection (e.g., of a wound, dermatitis, scabies, diabetic ulcers etc.), tropical ulcers, blistering distal dactylitis, streptococcal perianal and/or vulval dermatitis. In some cases, a disease or condition comprises atopic dermatitis, seborrheic dermatitis, inflammation, eczema, psoriasis, rosacea, mycoses, dermatophytosis, folliculitis, acne, alopecia, vitiligo, dandruff, chronic wound, skin ulcer, Netherton syndrome, hidradenitis suppurativa, sycosis vulgaris, staphylococcal scalded skin syndrome, impetigo, ecthyma, cellulitis, carbuncle, furuncle, and abscess. In some instances, a genetic condition comprises Netherton syndrome. In some instances, an inflammatory condition comprises atopic dermatitis, contact dermatitis, eczema, pruritus, seborrheic dermatitis, acne (acne vulgaris), psoriasis (psoriasis vulgaris, plaque psoriasis), rosacea, hidradenitis suppurativa. In some cases, a skin condition comprises an aging condition, such as wrinkles. In some cases, a skin condition comprises dandruff, or a sunburn. In some cases, a skin disease or condition comprises eczema, diaper rash, seborrheic dermatitis, chickenpox, measles, warts, acne, fifth disease, hives, ringworm, rashes from a bacterial or a fungal infections (e.g., cutaneous candidiasis), or rashes from an allergic reaction.

In some embodiments, a composition described herein is administered to treat a vaginal condition. Vaginal conditions and diseases for treatment following administration of a composition described herein can include vulvovaginal candidiasis, recurrent vulvovaginal candidiasis, and bacterial vaginosis.

In some embodiments, provided herein are methods of preventing or reducing a skin condition in a subject, comprising topically administering to a subject a composition described herein. In some cases, compositions and methods described herein are compositions used to improve a cosmetic irregularity. In some cases, compositions and methods described herein are compositions used to treat a condition selected from the group consisting of: a pruritis, an aesthetic condition, and a body odor. In some cases, an aesthetic condition comprises wrinkles or appearance of aging. In some instances, an aesthetic condition is a cosmetic irregularity. Improvement of the cosmetic irregularity may result in reduced fine line occurrence, fine line depth, wrinkle occurrence, wrinkle depth, scaly patches, roughness, acne, scars, irregular pigmentation, sun spots, liver spots, solar lentigines, melasma, poikiloderma, actinic keratoses, lentigo maligna, periorbital hyperpigmentation, shine, sheen, oily appearance, blistering, peeling, sloughing, flaking, or pore size; improved skin tone, skin firmness, tactile smoothness, suppleness, glow, visual smoothness, or radiance. In addition, improvement of the cosmetic irregularity with a composition or method described herein may reduce a pathogenic bacteria, virus or fungi on and/or in the skin of a subject in need thereof. In some cases, compositions and methods described herein provide for enhanced barrier function of the skin as measured by trans-epidermal water loss. Administrations described herein, e.g., topical, oral, or rectal, may reduce reoccurrences, so that additional incidents of the cosmetic irregularities of the skin are reduced in number, intensity, or frequency. The administration may increase the time of remission, such as the length of time between incidents. In some embodiments, an additional incident of cosmetic irregularity does not occur for 1, 2, 3, 4, 5, 6, 7, 8, 9, 10, 11, or 12 weeks following application. In some embodiments, an additional incident of cosmetic irregularity does not occur for 1, 2, 3, 4, 5, 6, 7, 8, 9, 10, 11, or 12 months following the topical application. For example, a composition herein is administered to reduce wrinkles, decrease lesion size, decrease pore size, or decrease pore density. Further provided herein are methods of preventing or reducing a skin condition in a subject, wherein the skin condition comprises fine line occurrence, fine line depth, wrinkle occurrence, wrinkle depth, nasolabial folds, scaly patches, roughness, acne, scars, redness, irregular pigmentation, decreased tightness, decreased skin barrier, age spots, sun spots, liver spots, photo damage, sunlight exposure, damage from sunlight exposure, environmental damage, solar lentigines, melasma, poikiloderma, actinic keratoses, lentigo maligna, periorbital hyperpigmentation, shine, sheen, oily appearance, decrease of skin's ability to retain moisture, abnormal skin epidermal thickness, reduction of dermal epidermal junction, blistering, peeling, sloughing, flaking, pore size, skin tone, skin firmness, skin texture, skin elasticity, tactile smoothness, suppleness, glow, visual smoothness, radiance or a combination thereof. In some cases, efficacy assessments of skin conditions and/or cosmetic irregularity to a treatment can be clinically graded using a ten-point scale, on a subject's treatment area (e.g., a subject's face) (where 0=none, 0.5-3.5=mild, 4-6.5=moderate and 7-10=severe). In some cases, efficacy assessments can be taken over a period of time, for example, after each treatment, or every week, or every month. All grading assessments can be performed by the same investigator at each visit or by a computer implementing an algorithm to ensure grading consistency. In some cases, a measurement is taken, such as the number or wrinkles, or the depth of wrinkles, and is a factor in a clinical score.

In some embodiments, a symptom of inflammation is treated by administering a composition described herein. In some embodiments, a symptom of a skin condition or disease is treated by administering a composition described herein. For example, a composition can be administered in an amount sufficient to reduce symptoms associated with atopic dermatitis. In some cases, a symptom comprises a raised bump, a discolored bump, a rash, a pain, an itch, a scaly skin, a rough skin, a peeling skin, an ulcer, an open sore, a lesion, a dry skin, a cracked skin, a discolored patch of skin, a fleshy bump, a wart, a skin growth, a mole, a change in a mole size or color, a loss of skin pigment, or an excessive flushing. In some cases, a symptom comprises an itch, a rash, a redness, a pain, a swelling, a blistering, or a scaling. In some embodiments, a composition is administered in an amount sufficient to reduce symptoms associated with dry skin. In some cases, a composition is administered in an amount sufficient for a reduction of incidence of a condition associated with inflammation, for example an itch, a rash, a redness, a pain, a swelling, a blistering, or a scaling.

In some embodiments, a method for the treatment of inflammation comprises topically administering a composition comprising Bacillus wiedmannii to a subject in need thereof. In some cases, the Bacillus wiedmannii is purified, and the Bacillus wiedmannii is present in an amount sufficient for treatment of inflammation. In some embodiments, a method comprises reducing growth of S. aureus on the skin of a subject in need thereof. In some cases, the method comprises topically administering a pharmaceutical composition comprising Bacillus velezensis, Bacillus wiedmannii, and Lysinibacillus macroides to a skin of the subject in need thereof. In some cases, the Bacillus velezensis, the Bacillus wiedmannii, and the Lysinibacillus macroides are purified, and the Bacillus velezensis, the Bacillus wiedmannii, and the Lysinibacillus macroides are viable, and are present in an amount sufficient for reduction of S. aureus on a skin of the subject in need thereof. In some embodiments, a method for the reduction of symptoms associated with atopic dermatitis comprises topically administering a composition comprising Bacillus amyloliquefaciens to a subject in need thereof. In some cases, the Bacillus amyloliquefaciens is purified, and the Bacillus amyloliquefaciens is present in an amount sufficient for the reduction of symptoms associated with atopic dermatitis. In some embodiments, a method for reducing the incidence of a condition associated with inflammation comprises topically administering a composition comprising Bacillus wiedmannii and Staphylococcus caprae to a subject in need thereof. In some cases, the Bacillus wiedmannii and the Staphylococcus caprae are purified. In some cases, the Bacillus wiedmannii and the Staphylococcus caprae are present in an amount sufficient for a reduction of the incidence of a condition associated with inflammation. In some instances, the condition associated with inflammation comprises an itch, a rash, a redness, a pain, a swelling, a blistering, or a scaling.

In some embodiments, a method for the treatment of inflammation comprises topically administering a composition comprising bacterial Strain jl.27 to a subject in need thereof. In some cases, the Strain jl.27 is purified, and the Strain jl.27 is present in an amount sufficient for treatment of inflammation. In some embodiments, a method comprises reducing growth of S. aureus on the skin of a subject in need thereof. In some cases, the method comprises topically administering a pharmaceutical composition comprising Strain jl.83, Strain jl.27, and Strain jl.77 to a skin of the subject in need thereof. In some cases, the Strain jl.83, the Strain jl.27 and the Strain jl.77 are purified, and the Strain jl.83, the Strain jl.27, and the Strain jl.77 are viable, and are present in an amount sufficient for reduction of S. aureus on a skin of the subject in need thereof. In some embodiments, a method for the reduction of symptoms associated with atopic dermatitis comprises topically administering a composition comprising Strain jl.68 to a subject in need thereof. In some cases, the Strain jl.68 is purified, and the Strain jl.68 is present in an amount sufficient for the reduction of symptoms associated with atopic dermatitis. In some embodiments, a method for reducing the incidence of a condition associated with inflammation comprises topically administering a composition comprising Strain jl.27 and Strain jl.121 to a subject in need thereof. In some cases, the Strain jl.27 and the Strain jl.121 are purified. In some cases, the Strain jl.27 and the Strain jl.121 are present in an amount sufficient for a reduction of the incidence of a condition associated with inflammation. In some instances, the condition associated with inflammation comprises an itch, a rash, a redness, a pain, a swelling, a blistering, or a scaling.

### Kits

Pharmaceutical compositions described herein may include kits where bacteria described herein are included in a first container (e.g., lyophilized cells), and one or more pharmaceutical acceptable excipients are included in a second container (e.g., water or a buffered solution). In some embodiments, provided herein are kits, wherein the kit comprises: a first container, wherein the first container comprises a purified, and lyophilized bacteria that comprises: Staphylococcus cohnii, Staphylococcus capitis, Staphylococcus caprae, Bacillus tropicus, Bacillus mycoides, Bacillus wiedmannii, Bacillus mediterraneensis, Bacillus amyloliquefaciens, Bacillus velezensis, Bacillus cereus, Bacillus cecembensis, Kocuria marina, Lysinibacillus macrolides, or a mixture thereof; and a second container, wherein the second container comprises a pharmaceutically acceptable excipient. In some embodiments, provided herein are kits, wherein the kit comprises: a first container, wherein the first container comprises a purified, and lyophilized bacteria that comprises: Strain jl.83, Strain jl.27, Strain jl.77, Strain jl.68, Strain jl.121, Strain jl.21, or a mixture thereof; and a second container, wherein the second container comprises a pharmaceutically acceptable excipient. In some embodiments, provided herein are kits, wherein the kit comprises: a first container, wherein the first container comprises a purified, and lyophilized bacteria that comprises: Bacillus velezensis, Bacillus wiedmannii, and Lysinibacillus macrolides; and a second container, wherein the second container comprises a pharmaceutically acceptable excipient. In some embodiments, provided herein are kits, wherein the kit comprises: a first container, wherein the first container comprises a purified, and lyophilized bacteria that comprises: Bacillus amyloliquefaciens, Bacillus wiedmannii, and Staphylococcus caprae; and a second container, wherein the second container comprises a pharmaceutically acceptable excipient. In some embodiments, provided herein are kits, wherein the kit comprises: a first container, wherein the first container comprises a purified, and lyophilized bacteria that comprises: Bacillus amyloliquefaciens; Bacillus tropicus; and Staphylococcus caprae; and a second container, wherein the second container comprises a pharmaceutically acceptable excipient.

In some embodiments, provided herein are kits, wherein the kit comprises: a first container, wherein the first container comprises a purified, and lyophilized bacteria that comprises: Strain jl.83, Strain jl.27, and Strain jl.77; and a second container, wherein the second container comprises a pharmaceutically acceptable excipient. In some embodiments, provided herein are kits, wherein the kit comprises: a first container, wherein the first container comprises a purified, and lyophilized bacteria that comprises: Strain jl.68, Strain jl.27, and Strain jl.121; and a second container, wherein the second container comprises a pharmaceutically acceptable excipient. In some embodiments, provided herein are kits, wherein the kit comprises: a first container, wherein the first container comprises a purified, and lyophilized bacteria that comprises: Strain jl.68; Strain jl.21; and Strain jl.121; and a second container, wherein the second container comprises a pharmaceutically acceptable excipient.

In some embodiments, provided herein are kits, wherein the kit comprises: a first container, wherein the first container comprises a purified, and lyophilized bacteria present in a total amount of at least 10\hat{ }3 cfu that comprises: one or more species from Table 1 and/or one or more strains from Table 2; and a second container, wherein the second container comprises a pharmaceutically acceptable excipient. In some instances, a kit comprises 1, 2, 3, 4, 5, or more containers, such that each container comprises an individual strain or species of bacteria. Further provided herein are kits, wherein the purified, and lyophilized bacteria are present in a total amount of up to 10\hat{ }15 cfu. Further provided herein are kits, wherein the purified, and lyophilized bacteria are present in a total amount of 10\hat{ }3 to 10\hat{ }12 cfu.

### NUMBERED EMBODIMENTS

A number of compositions, and methods are disclosed herein. Specific exemplary embodiments of these compositions and methods are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1. A composition, wherein the composition comprises: bacterial species that are purified, wherein the bacterial species comprise: Bacillus velezensis, Bacillus wiedmannii, and Lysinibacillus macroides; and wherein (a) the bacterial species are lyophilized; or (b) the composition is formulated for delivery to a skin.

Embodiment 2. The composition of embodiment 1, wherein the composition is formulated as: a suspension, an emulsion, a cream, a lotion, a tincture, a gel, a foam, a powder, an ointment, a paste, or an oil.

Embodiment 3. The composition of embodiment 1, further comprising a fourth bacterial species.

Embodiment 4. A composition, wherein the composition comprises: bacterial species that are purified, wherein the bacterial species comprise: Bacillus amyloliquefaciens; Bacillus wiedmannii; and Staphylococcus caprae; and wherein (a) the bacterial species are lyophilized; or (b) the composition is formulated for delivery to a skin.

Embodiment 5. The composition of embodiment 4, wherein the composition is formulated as: a suspension, an emulsion, a cream, a lotion, a tincture, a gel, a foam, a powder, an ointment, a paste, or an oil.

Embodiment 6. The composition of embodiment 4, further comprising a fourth bacterial species.

Embodiment 7. A composition, wherein the composition comprises: bacterial species that are purified, wherein the bacterial species comprise: Bacillus amyloliquefaciens; Bacillus tropicus; and Staphylococcus caprae; and wherein (a) the bacterial species are lyophilized; or (b) the composition is formulated for delivery to a skin.

Embodiment 8. The composition of embodiment 7, wherein the composition is formulated as: a suspension, an emulsion, a cream, a lotion, a tincture, a gel, a foam, a powder, an ointment, a paste, or an oil.

Embodiment 9. The composition of embodiment 7, further comprising a fourth bacterial species.

Embodiment 10. A composition, wherein the composition comprises: bacterial species that are purified, wherein the bacterial species comprise at least two of the following bacterial species: Bacillus velezensis, Bacillus wiedmannii, Lysinibacillus macrolides, Bacillus amyloliquefaciens, Staphylococcus caprae, and Bacillus tropicus, and wherein (a) the at least two bacterial species are lyophilized; or (b) the composition is formulated for delivery to a skin.

Embodiment 11. The composition of embodiment 10, wherein the composition is formulated as: a suspension, an emulsion, a cream, a lotion, a tincture, a gel, a foam, a powder, an ointment, a paste, or an oil.

Embodiment 12. A composition, wherein the composition comprises: a bacterial species that is purified, wherein the bacterial species comprises: Bacillus velezensis; and wherein (a) the bacterial species is lyophilized; or (b) the composition is formulated for delivery to a skin.

Embodiment 13. The composition of embodiment 12, wherein the composition is formulated as: a suspension, an emulsion, a cream, a lotion, a tincture, a gel, a foam, a powder, an ointment, a paste, or an oil.

Embodiment 14. The composition of embodiment 12, further comprising a second bacterial species.

Embodiment 15. A composition, wherein the composition comprises: a bacterial species that is purified, wherein the bacterial species comprises: Bacillus wiedmannii; and wherein (a) the bacterial species is lyophilized; or (b) the composition is formulated for delivery to a skin.

Embodiment 16. The composition of embodiment 15, wherein the composition is formulated as: a suspension, an emulsion, a cream, a lotion, a tincture, a gel, a foam, a powder, an ointment, a paste, or an oil.

Embodiment 17. The composition of embodiment 15, further comprising a second bacterial species.

Embodiment 18. A composition, wherein the composition comprises: a bacterial species that is purified, wherein the bacterial species comprises: Lysinibacillus macroides; and wherein (a) the bacterial species is lyophilized; or (b) the composition is formulated for delivery to a skin.

Embodiment 19. The composition of embodiment 18, wherein the composition is formulated as: a suspension, an emulsion, a cream, a lotion, a tincture, a gel, a foam, a powder, an ointment, a paste, or an oil.

Embodiment 20. The composition of embodiment 18, further comprising a second bacterial species.

Embodiment 21. A composition, wherein the composition comprises: a bacterial species that is purified, wherein the bacterial species comprises: Bacillus tropicus; and wherein (a) the bacterial species is lyophilized; or (b) the composition is formulated for delivery to a skin.

Embodiment 22. The composition of embodiment 21, wherein the composition is formulated as: a suspension, an emulsion, a cream, a lotion, a tincture, a gel, a foam, a powder, an ointment, a paste, or an oil.

Embodiment 23. The composition of embodiment 21, further comprising a second bacterial species.

Embodiment 24. The composition of any one of embodiments 1-23, wherein the composition comprises at least 10\hat{ }3 colony forming units (cfu) per gram of bacteria.

Embodiment 25. The composition of any one of embodiments 1-24, wherein the composition comprises 10\hat{ }3 to 10\hat{ }12 colony forming units (cfu) per gram of bacteria.

Embodiment 26. The composition of any one of embodiments 1-25, wherein the bacterial species are grown in aerobic conditions.

Embodiment 27. The composition of any one of embodiments 1-26, wherein the bacterial species are grown without animal products.

Embodiment 28. The composition of any one of embodiments 1-27, wherein the bacterial species are grown in Tryptic Soy Broth (TSB).

Embodiment 29. The composition of any one of embodiments 1-28, wherein the composition when stored in a sealed container placed at 20° C. retains greater than about: 10\hat{ }4 cfu after 6 months, as measured by cfu counts.

Embodiment 30. The composition of any one of embodiments 1-29, further comprising an excipient.

Embodiment 31. The composition of any one of embodiments 1-30, further comprising a lyoprotectant.

Embodiment 32. The composition of any one of embodiments 1-31, further comprising an emollient.

Embodiment 33. The composition of any one of embodiments 1-32, further comprising thiamine or a salt thereof.

Embodiment 34. The composition of any one of embodiments 1-33, wherein the bacterial species when contacted with S. aureus cause a reduction in expression of at least one of the following genes: gmk, agr, psma, saeR, ccpA, and SigB in S. aureus, wherein the reduction in expression is measured by a fluorescence reporter assay.

Embodiment 35. The composition of any one of embodiments 1-33, wherein the bacterial species is present in an amount effective to suppress virulence of S. aureus as measured by a reduction in expression of at least one of the following genes: gmk, agr, psma, saeR, ccpA, and SigB in S. aureus wherein the reduction in expression is measured by a fluorescence reporter assay.

Embodiment 36. A method of administering the composition of any one of embodiments 1-35, comprising administering an amount sufficient to treat a disease selected from the group consisting of: atopic dermatitis, seborrheic dermatitis, inflammation, eczema, psoriasis, rosacea, mycoses, dermatophytosis, folliculitis, acne, alopecia, vitiligo, dandruff, chronic wound, skin ulcer, Netherton syndrome, hidradenitis suppurativa, sycosis vulgaris, staphylococcal scalded skin syndrome, impetigo, ecthyma, cellulitis, carbuncle, furuncle, and abscess.

Embodiment 37. A method of administering the composition of any one of embodiments 1-35, comprising administering an amount sufficient to reduce symptoms associated with atopic dermatitis.

Embodiment 38. A method of administering the composition of any one of embodiments 1-35, comprising administering the composition to a subject who has an eczema prone microbiome prior to the administration.

Embodiment 39. A method of administering the composition of any one of embodiments 1-35, comprising administering an amount sufficient to reduce symptoms associated with dry skin.

Embodiment 40. A method of administering the composition of any one of embodiments 1-35, comprising administering an amount sufficient for a reduction of incidence of a condition associated with inflammation and wherein the condition associated with inflammation comprises an itch, a rash, a redness, a pain, a swelling, a blistering, or a scaling.

Embodiment 41. A method of administering the composition of any one of embodiments 1-35, comprising administering an amount sufficient to suppress virulence of S. aureus as measured by a reduction in expression of at least one of the following genes: gmk, agr, psma, saeR, ccpA, and SigB in S. aureus, wherein the reduction in expression is measured by a fluorescence reporter assay.

Embodiment 42. A composition of any one of embodiments 1-35, for use in the treatment of a skin condition.

Embodiment 43. A composition of any one of embodiments 1-35, for use in the treatment of inflammation.

Embodiment 44. A method for treatment of inflammation, comprising: topically administering a composition comprising Bacillus wiedmannii to a subject in need thereof, wherein the Bacillus wiedmannii is purified, and wherein the Bacillus wiedmannii is present in an amount sufficient for treatment of inflammation.

Embodiment 45. A method for reducing growth of Staphylococcus aureus on skin of a subject in need thereof, comprising: topically administering a pharmaceutical composition comprising Bacillus velezensis, Bacillus wiedmannii, and Lysinibacillus macroides to a skin of the subject in need thereof, wherein the Bacillus velezensis, the Bacillus wiedmannii, and the Lysinibacillus macroides are purified, and wherein the Bacillus velezensis, the Bacillus wiedmannii, and the Lysinibacillus macroides are viable, and present in an amount sufficient for reduction of S. aureus on a skin of the subject in need thereof.

Embodiment 46. A method for reduction of symptoms associated with atopic dermatitis, comprising: topically administering a composition comprising Bacillus amyloliquefaciens to a subject in need thereof, wherein the Bacillus amyloliquefaciens is purified, and wherein the Bacillus amyloliquefaciens is present in an amount sufficient for the reduction of symptoms associated with atopic dermatitis.

Embodiment 47. A method for reducing an incidence of a condition associated with inflammation comprising: topically administering a composition comprising Bacillus wiedmannii and Staphylococcus caprae to a subject in need thereof, wherein the Bacillus wiedmannii and the Staphylococcus caprae are purified, wherein the Bacillus wiedmannii and the Staphylococcus caprae are present in an amount sufficient for a reduction of the incidence of a condition associated with inflammation and wherein the condition associated with inflammation comprises an itch, a rash, a redness, a pain, a swelling, a blistering, or a scaling.

Embodiment 48. A method of administering the composition of any one of embodiments 1-35, comprising administering an amount sufficient to treat a condition selected from the group consisting of: pruritis, an aesthetic condition, and body odor.

Embodiment 49. The method of embodiment 48, wherein the aesthetic condition comprises wrinkles or appearance of aging.

### Summary of Embodiments

Disclosed herein are compositions. In some embodiments, a composition comprises bacterial species that are purified. In some embodiments, the bacterial species comprises: Bacillus velezensis; Bacillus wiedmannii; and Lysinibacillus macroides. In some embodiments (a) the bacterial species are lyophilized; or (b) the composition is formulated for delivery to a skin. In some embodiments, the composition is formulated as: a suspension, an emulsion, a cream, a lotion, a tincture, a gel, a foam, a powder, an ointment, a paste, or an oil. In some embodiments, the composition can further comprise a fourth bacterial species. In some embodiments, the composition comprises at least 10\hat{ }3 colony forming units (cfu) per gram of bacteria. In some embodiments, the composition comprises 10\hat{ }3 to 10\hat{ }12 colony forming units (cfu) per gram of bacteria. In some embodiments, the bacterial species are grown in aerobic conditions. In some embodiments, the bacterial species are grown without animal products. In some embodiments, the bacterial species are grown in Tryptic Soy Broth (TSB). In some embodiments, the composition when stored in a sealed container placed at 20° C. can retain greater than about 10\hat{ }4 cfu after 6 months, as measured by cfu counts. In some embodiments, the composition can further comprise an excipient. In some embodiments, the composition can further comprise a lyoprotectant. In some embodiments, the composition can further comprise an emollient. In some embodiments, the composition can further comprise thiamine or a salt thereof. In some embodiments, the bacterial species when contacted with S. aureus can cause a reduction in expression of at least one of the following genes: gmk, agr, psma, saeR, ccpA, and SigB in S. aureus. In some embodiments, the reduction in expression is measured by a fluorescence reporter assay. In some embodiments, the bacterial species are present in an amount effective to suppress virulence of S. aureus as measured by a reduction in expression of at least one of the following genes: gmk, agr, psma, saeR, ccpA, and SigB in S. aureus. In some embodiments, the reduction in expression is measured by a fluorescence reporter assay. Also disclosed herein are methods of administering the composition. In some embodiments, the method comprises administering an amount sufficient to treat a disease selected from the group consisting of: atopic dermatitis, seborrheic dermatitis, inflammation, eczema, psoriasis, rosacea, mycoses, dermatophytosis, folliculitis, acne, alopecia, vitiligo, dandruff, chronic wound, skin ulcer, Netherton syndrome, hidradenitis suppurativa, sycosis vulgaris, staphylococcal scalded skin syndrome, impetigo, ecthyma, cellulitis, carbuncle, furuncle, and abscess. In some embodiments, the method comprises administering an amount sufficient to treat a condition selected from the group consisting of: pruritis, an aesthetic condition, and body odor. In some embodiments, the aesthetic condition comprises wrinkles or appearance of aging. In some embodiments, the method comprises administering an amount sufficient to reduce symptoms associated with atopic dermatitis. In some embodiments, the method comprises administering the composition to a subject who has an eczema prone microbiome prior to the administration. In some embodiments, the method comprises administering an amount sufficient to reduce symptoms associated with dry skin. In some embodiments, the method comprises administering an amount sufficient for a reduction of incidence of a condition associated with inflammation and wherein the condition associated with inflammation comprises an itch, a rash, a redness, a pain, a swelling, a blistering, or a scaling. In some embodiments, the method comprises administering an amount sufficient to suppress virulence of S. aureus as measured by a reduction in expression of at least one of the following genes: gmk, agr, psma, saeR, ccpA, and SigB in S. aureus. In some embodiments, the reduction in expression is measured by a fluorescence reporter assay. In some embodiments, the composition is for use in the treatment of a skin condition. In some embodiments, the composition is for use in the treatment of inflammation.

In some embodiments, a composition comprises bacterial species that are purified. In some embodiments, the bacterial species comprises: Bacillus amyloliquefaciens, Bacillus wiedmannii, and Staphylococcus caprae. In some embodiments, (a) the bacterial species are lyophilized; or (b) the composition is formulated for delivery to a skin. In some embodiments, a composition is formulated as: a suspension, an emulsion, a cream, a lotion, a tincture, a gel, a foam, a powder, an ointment, a paste, or an oil. In some embodiments, the composition can further comprise a fourth bacterial species. In some embodiments, the composition comprises at least 10\hat{ }3 colony forming units (cfu) per gram of bacteria. In some embodiments, the composition comprises 10\hat{ }3 to 10\hat{ }12 colony forming units (cfu) per gram of bacteria. In some embodiments, the bacterial species are grown in aerobic conditions. In some embodiments, the bacterial species are grown without animal products. In some embodiments, the bacterial species are grown in Tryptic Soy Broth (TSB). In some embodiments, the composition when stored in a sealed container placed at 20° C. can retain greater than about 10\hat{ }4 cfu after 6 months, as measured by cfu counts. In some embodiments, the composition can further comprise an excipient. In some embodiments, the composition can further comprise a lyoprotectant. In some embodiments, the composition can further comprise an emollient. In some embodiments, the composition can further comprise thiamine or a salt thereof. In some embodiments, the bacterial species when contacted with S. aureus can cause a reduction in expression of at least one of the following genes: gmk, agr, psma, saeR, ccpA, and SigB in S. aureus. In some embodiments, the reduction in expression is measured by a fluorescence reporter assay. In some embodiments, the bacterial species are present in an amount effective to suppress virulence of S. aureus as measured by a reduction in expression of at least one of the following genes: gmk, agr, psma, saeR, ccpA, and SigB in S. aureus. In some embodiments, the reduction in expression is measured by a fluorescence reporter assay. Also disclosed herein are methods of administering the composition. In some embodiments, the method comprises administering an amount sufficient to treat a disease selected from the group consisting of: atopic dermatitis, seborrheic dermatitis, inflammation, eczema, psoriasis, rosacea, mycoses, dermatophytosis, folliculitis, acne, alopecia, vitiligo, dandruff, chronic wound, skin ulcer, Netherton syndrome, hidradenitis suppurativa, sycosis vulgaris, staphylococcal scalded skin syndrome, impetigo, ecthyma, cellulitis, carbuncle, furuncle, and abscess. In some embodiments, the method comprises administering an amount sufficient to treat a condition selected from the group consisting of: pruritis, an aesthetic condition, and body odor. In some embodiments, the aesthetic condition comprises wrinkles or appearance of aging. In some embodiments, the method comprises administering an amount sufficient to reduce symptoms associated with atopic dermatitis. In some embodiments, the method comprises administering the composition to a subject who has an eczema prone microbiome prior to the administration. In some embodiments, the method comprises administering an amount sufficient to reduce symptoms associated with dry skin. In some embodiments, the method comprises administering an amount sufficient for a reduction of incidence of a condition associated with inflammation and wherein the condition associated with inflammation comprises an itch, a rash, a redness, a pain, a swelling, a blistering, or a scaling. In some embodiments, the method comprises administering an amount sufficient to suppress virulence of S. aureus as measured by a reduction in expression of at least one of the following genes: gmk, agr, psma, saeR, ccpA, and SigB in S. aureus. In some embodiments, the reduction in expression is measured by a fluorescence reporter assay. In some embodiments, the composition is for use in the treatment of a skin condition. In some embodiments, the composition is for use in the treatment of inflammation.

In some embodiments, a composition comprises bacterial species that are purified. In some embodiments, the bacterial species comprises: Bacillus amyloliquefaciens, Bacillus tropicus, and Staphylococcus caprae. In some embodiments, (a) the bacterial species are lyophilized; or (b) the composition is formulated for delivery to a skin. In some embodiments, the composition is formulated as: a suspension, an emulsion, a cream, a lotion, a tincture, a gel, a foam, a powder, an ointment, a paste, or an oil. In some embodiments, the composition can further comprise a fourth bacterial species. In some embodiments, the composition comprises at least 10\hat{ }3 colony forming units (cfu) per gram of bacteria. In some embodiments, the composition comprises 10\hat{ }3 to 10\hat{ }12 colony forming units (cfu) per gram of bacteria. In some embodiments, the bacterial species are grown in aerobic conditions. In some embodiments, the bacterial species are grown without animal products. In some embodiments, the bacterial species are grown in Tryptic Soy Broth (TSB). In some embodiments, the composition when stored in a sealed container placed at 20° C. can retain greater than about 10\hat{ }4 cfu after 6 months, as measured by cfu counts. In some embodiments, the composition can further comprise an excipient. In some embodiments, the composition can further comprise a lyoprotectant. In some embodiments, the composition can further comprise an emollient. In some embodiments, the composition can further comprise thiamine or a salt thereof. In some embodiments, the bacterial species when contacted with S. aureus can cause a reduction in expression of at least one of the following genes: gmk, agr, psma, saeR, ccpA, and SigB in S. aureus. In some embodiments, the reduction in expression is measured by a fluorescence reporter assay. In some embodiments, the bacterial species is present in an amount effective to suppress virulence of S. aureus as measured by a reduction in expression of at least one of the following genes: gmk, agr, psma, saeR, ccpA, and SigB in S. aureus. In some embodiments, the reduction in expression is measured by a fluorescence reporter assay. Also disclosed herein are methods of administering the composition. In some embodiments, the method comprises administering an amount sufficient to treat a disease selected from the group consisting of: atopic dermatitis, seborrheic dermatitis, inflammation, eczema, psoriasis, rosacea, mycoses, dermatophytosis, folliculitis, acne, alopecia, vitiligo, dandruff, chronic wound, skin ulcer, Netherton syndrome, hidradenitis suppurativa, sycosis vulgaris, staphylococcal scalded skin syndrome, impetigo, ecthyma, cellulitis, carbuncle, furuncle, and abscess. In some embodiments, the method comprises administering an amount sufficient to treat a condition selected from the group consisting of: pruritis, an aesthetic condition, and body odor. In some embodiments, the aesthetic condition comprises wrinkles or appearance of aging. In some embodiments, the method comprises administering an amount sufficient to reduce symptoms associated with atopic dermatitis. In some embodiments, the method comprises administering the composition to a subject who has an eczema prone microbiome prior to the administration. In some embodiments, the method comprises administering an amount sufficient to reduce symptoms associated with dry skin. In some embodiments, the method comprises administering an amount sufficient for a reduction of incidence of a condition associated with inflammation and wherein the condition associated with inflammation comprises an itch, a rash, a redness, a pain, a swelling, a blistering, or a scaling. In some embodiments, the method comprises administering an amount sufficient to suppress virulence of S. aureus as measured by a reduction in expression of at least one of the following genes: gmk, agr, psma, saeR, ccpA, and SigB in S. aureus. In some embodiments, the reduction in expression is measured by a fluorescence reporter assay. In some embodiments, the composition is for use in the treatment of a skin condition. In some embodiments, the composition is for use in the treatment of inflammation.

In some embodiments, a composition comprises bacterial species that are purified. In some embodiments, the bacterial species comprises at least two of the following bacterial species: Bacillus velezensis, Bacillus wiedmannii, Lysinibacillus macrolides, Bacillus amyloliquefaciens, Staphylococcus caprae, and Bacillus tropicus. In some embodiments, (a) the at least two bacterial species are lyophilized; or (b) the composition is formulated for delivery to a skin. In some embodiments, the composition is formulated as: a suspension, an emulsion, a cream, a lotion, a tincture, a gel, a foam, a powder, an ointment, a paste, or an oil. In some embodiments, the composition comprises at least 10\hat{ }3 colony forming units (cfu) per gram of bacteria. In some embodiments, the composition comprises 10\hat{ }3 to 10\hat{ }12 colony forming units (cfu) per gram of bacteria. In some embodiments, the bacterial species are grown in aerobic conditions. In some embodiments, the bacterial species are grown without animal products. In some embodiments, the bacterial species are grown in Tryptic Soy Broth (TSB). In some embodiments, the composition when stored in a sealed container placed at 20° C. can retain greater than about 10\hat{ }4 cfu after 6 months, as measured by cfu counts. In some embodiments, the composition can further comprise an excipient. In some embodiments, the composition can further comprise a lyoprotectant. In some embodiments, the composition can further comprise an emollient. In some embodiments, the composition can further comprise thiamine or a salt thereof. In some embodiments, the bacterial species when contacted with S. aureus can cause a reduction in expression of at least one of the following genes: gmk, agr, psma, saeR, ccpA, and SigB in S. aureus. In some embodiments, the reduction in expression is measured by a fluorescence reporter assay. In some embodiments, the bacterial species are present in an amount effective to suppress virulence of S. aureus as measured by a reduction in expression of at least one of the following genes: gmk, agr, psma, saeR, ccpA, and SigB in S. aureus. In some embodiments, the reduction in expression is measured by a fluorescence reporter assay. Also disclosed herein are methods of administering the composition. In some embodiments, the method comprises administering an amount sufficient to treat a disease selected from the group consisting of: atopic dermatitis, seborrheic dermatitis, inflammation, eczema, psoriasis, rosacea, mycoses, dermatophytosis, folliculitis, acne, alopecia, vitiligo, dandruff, chronic wound, skin ulcer, Netherton syndrome, hidradenitis suppurativa, sycosis vulgaris, staphylococcal scalded skin syndrome, impetigo, ecthyma, cellulitis, carbuncle, furuncle, and abscess. In some embodiments, the method comprises administering an amount sufficient to treat a condition selected from the group consisting of: pruritis, an aesthetic condition, and body odor. In some embodiments, the aesthetic condition comprises wrinkles or appearance of aging. In some embodiments, the method comprises administering an amount sufficient to reduce symptoms associated with atopic dermatitis. In some embodiments, the method comprises administering the composition to a subject who has an eczema prone microbiome prior to the administration. In some embodiments, the method comprises administering an amount sufficient to reduce symptoms associated with dry skin. In some embodiments, the method comprises administering an amount sufficient for a reduction of incidence of a condition associated with inflammation and wherein the condition associated with inflammation comprises an itch, a rash, a redness, a pain, a swelling, a blistering, or a scaling. In some embodiments, the method comprises administering an amount sufficient to suppress virulence of S. aureus as measured by a reduction in expression of at least one of the following genes: gmk, agr, psma, saeR, ccpA, and SigB in S. aureus. In some embodiments, the reduction in expression is measured by a fluorescence reporter assay. In some embodiments, the composition is for use in the treatment of a skin condition. In some embodiments, the composition is for use in the treatment of inflammation.

In some embodiments, a composition comprises a bacterial species that is purified. In some embodiments, the bacterial species comprises: Bacillus velezensis. In some embodiments, (a) the bacterial species is lyophilized; or (b) the composition is formulated for delivery to a skin. In some embodiments, the composition is formulated as: a suspension, an emulsion, a cream, a lotion, a tincture, a gel, a foam, a powder, an ointment, a paste, or an oil. In some embodiments, the composition can further comprise a second bacterial species. In some embodiments, the composition comprises at least 10\hat{ }3 colony forming units (cfu) per gram of bacteria. In some embodiments, the composition comprises 10\hat{ }3 to 10\hat{ }12 colony forming units (cfu) per gram of bacteria. In some embodiments, the bacterial species is grown in aerobic conditions. In some embodiments, the bacterial species is grown without animal products. In some embodiments, the bacterial species is grown in Tryptic Soy Broth (TSB). In some embodiments, the composition when stored in a sealed container placed at 20° C. can retain greater than about 10\hat{ }4 cfu after 6 months, as measured by cfu counts. In some embodiments, the composition can further comprise an excipient. In some embodiments, the composition can further comprise a lyoprotectant. In some embodiments, the composition can further comprise an emollient. In some embodiments, the composition can further comprise thiamine or a salt thereof. In some embodiments, the bacterial species when contacted with S. aureus can cause a reduction in expression of at least one of the following genes: gmk, agr, psma, saeR, ccpA, and SigB in S. aureus. In some embodiments, the reduction in expression is measured by a fluorescence reporter assay. In some embodiments, the bacterial species is present in an amount effective to suppress virulence of S. aureus as measured by a reduction in expression of at least one of the following genes: gmk, agr, psma, saeR, ccpA, and SigB in S. aureus. In some embodiments, the reduction in expression is measured by a fluorescence reporter assay. Also disclosed herein are methods of administering the composition. In some embodiments, the method comprises administering an amount sufficient to treat a disease selected from the group consisting of: atopic dermatitis, seborrheic dermatitis, inflammation, eczema, psoriasis, rosacea, mycoses, dermatophytosis, folliculitis, acne, alopecia, vitiligo, dandruff, chronic wound, skin ulcer, Netherton syndrome, hidradenitis suppurativa, sycosis vulgaris, staphylococcal scalded skin syndrome, impetigo, ecthyma, cellulitis, carbuncle, furuncle, and abscess. In some embodiments, the method comprises administering an amount sufficient to treat a condition selected from the group consisting of: pruritis, an aesthetic condition, and body odor. In some embodiments, the aesthetic condition comprises wrinkles or appearance of aging. In some embodiments, the method comprises administering an amount sufficient to reduce symptoms associated with atopic dermatitis. In some embodiments, the method comprises administering the composition to a subject who has an eczema prone microbiome prior to the administration. In some embodiments, the method comprises administering an amount sufficient to reduce symptoms associated with dry skin. In some embodiments, the method comprises administering an amount sufficient for a reduction of incidence of a condition associated with inflammation and wherein the condition associated with inflammation comprises an itch, a rash, a redness, a pain, a swelling, a blistering, or a scaling. In some embodiments, the method comprises administering an amount sufficient to suppress virulence of S. aureus as measured by a reduction in expression of at least one of the following genes: gmk, agr, psma, saeR, ccpA, and SigB in S. aureus. In some embodiments, the reduction in expression is measured by a fluorescence reporter assay. In some embodiments, the composition is for use in the treatment of a skin condition. In some embodiments, the composition is for use in the treatment of inflammation.

In some embodiments, a composition comprises a bacterial species that is purified. In some embodiments, the bacterial species comprises Bacillus wiedmannii. In some embodiments, (a) the bacterial species is lyophilized; or (b) the composition is formulated for delivery to a skin. In some embodiments, the composition is formulated as: a suspension, an emulsion, a cream, a lotion, a tincture, a gel, a foam, a powder, an ointment, a paste, or an oil. In some embodiments, the composition can further comprise a second bacterial species. In some embodiments, the composition comprises at least 10\hat{ }3 colony forming units (cfu) per gram of bacteria. In some embodiments, the composition comprises 10\hat{ }3 to 10\hat{ }12 colony forming units (cfu) per gram of bacteria. In some embodiments, the bacterial species is grown in aerobic conditions. In some embodiments, the bacterial species is grown without animal products. In some embodiments, the bacterial species is grown in Tryptic Soy Broth (TSB). In some embodiments, the composition when stored in a sealed container placed at 20° C. can retain greater than about 10\hat{ }4 cfu after 6 months, as measured by cfu counts. In some embodiments, the composition can further comprise an excipient. In some embodiments, the composition can further comprise a lyoprotectant. In some embodiments, the composition can further comprise an emollient. In some embodiments, the composition can further comprise thiamine or a salt thereof. In some embodiments, the bacterial species when contacted with S. aureus can cause a reduction in expression of at least one of the following genes: gmk, agr, psma, saeR, ccpA, and SigB in S. aureus. In some embodiments, the reduction in expression is measured by a fluorescence reporter assay. In some embodiments, the bacterial species is present in an amount effective to suppress virulence of S. aureus as measured by a reduction in expression of at least one of the following genes: gmk, agr, psma, saeR, ccpA, and SigB in S. aureus. In some embodiments, the reduction in expression is measured by a fluorescence reporter assay. Also disclosed herein are methods of administering the composition. In some embodiments, the method comprises administering an amount sufficient to treat a disease selected from the group consisting of: atopic dermatitis, seborrheic dermatitis, inflammation, eczema, psoriasis, rosacea, mycoses, dermatophytosis, folliculitis, acne, alopecia, vitiligo, dandruff, chronic wound, skin ulcer, Netherton syndrome, hidradenitis suppurativa, sycosis vulgaris, staphylococcal scalded skin syndrome, impetigo, ecthyma, cellulitis, carbuncle, furuncle, and abscess. In some embodiments, the method comprises administering an amount sufficient to treat a condition selected from the group consisting of: pruritis, an aesthetic condition, and body odor. In some embodiments, the aesthetic condition comprises wrinkles or appearance of aging. In some embodiments, the method comprises administering an amount sufficient to reduce symptoms associated with atopic dermatitis. In some embodiments, the method comprises administering the composition to a subject who has an eczema prone microbiome prior to the administration. In some embodiments, the method comprises administering an amount sufficient to reduce symptoms associated with dry skin. In some embodiments, the method comprises administering an amount sufficient for a reduction of incidence of a condition associated with inflammation and wherein the condition associated with inflammation comprises an itch, a rash, a redness, a pain, a swelling, a blistering, or a scaling. In some embodiments, the method comprises administering an amount sufficient to suppress virulence of S. aureus as measured by a reduction in expression of at least one of the following genes: gmk, agr, psma, saeR, ccpA, and SigB in S. aureus. In some embodiments, the reduction in expression is measured by a fluorescence reporter assay. In some embodiments, the composition is for use in the treatment of a skin condition. In some embodiments, the composition is for use in the treatment of inflammation.

In some embodiments, a composition comprises a bacterial species that is purified. In some embodiments, the bacterial species comprises: Lysinibacillus macroides. In some cases, (a) the bacterial species is lyophilized; or (b) the composition is formulated for delivery to a skin. In some embodiments, the composition is formulated as: a suspension, an emulsion, a cream, a lotion, a tincture, a gel, a foam, a powder, an ointment, a paste, or an oil. In some embodiments, the composition can further comprise a second bacterial species. In some embodiments, the composition comprises at least 10\hat{ }3 colony forming units (cfu) per gram of bacteria. In some embodiments, the composition comprises 10\hat{ }3 to 10\hat{ }12 colony forming units (cfu) per gram of bacteria. In some embodiments, the bacterial species is grown in aerobic conditions. In some embodiments, the bacterial species is grown without animal products. In some embodiments, the bacterial species is grown in Tryptic Soy Broth (TSB). In some embodiments, the composition when stored in a sealed container placed at 20° C. can retain greater than about 10\hat{ }4 cfu after 6 months, as measured by cfu counts. In some embodiments, the composition can further comprise an excipient. In some embodiments, the composition can further comprise a lyoprotectant. In some embodiments, the composition can further comprise an emollient. In some embodiments, the composition can further comprise thiamine or a salt thereof. In some embodiments, the bacterial species when contacted with S. aureus can cause a reduction in expression of at least one of the following genes: gmk, agr, psma, saeR, ccpA, and SigB in S. aureus. In some embodiments, the reduction in expression is measured by a fluorescence reporter assay. In some embodiments, the bacterial species is present in an amount effective to suppress virulence of S. aureus as measured by a reduction in expression of at least one of the following genes: gmk, agr, psma, saeR, ccpA, and SigB in S. aureus. In some embodiments, the reduction in expression is measured by a fluorescence reporter assay. Also disclosed herein are methods of administering the composition. In some embodiments, the method comprises administering an amount sufficient to treat a disease selected from the group consisting of: atopic dermatitis, seborrheic dermatitis, inflammation, eczema, psoriasis, rosacea, mycoses, dermatophytosis, folliculitis, acne, alopecia, vitiligo, dandruff, chronic wound, skin ulcer, Netherton syndrome, hidradenitis suppurativa, sycosis vulgaris, staphylococcal scalded skin syndrome, impetigo, ecthyma, cellulitis, carbuncle, furuncle, and abscess. In some embodiments, the method comprises administering an amount sufficient to treat a condition selected from the group consisting of: pruritis, an aesthetic condition, and body odor. In some embodiments, the aesthetic condition comprises wrinkles or appearance of aging. In some embodiments, the method comprises administering an amount sufficient to reduce symptoms associated with atopic dermatitis. In some embodiments, the method comprises administering the composition to a subject who has an eczema prone microbiome prior to the administration. In some embodiments, the method comprises administering an amount sufficient to reduce symptoms associated with dry skin. In some embodiments, the method comprises administering an amount sufficient for a reduction of incidence of a condition associated with inflammation and wherein the condition associated with inflammation comprises an itch, a rash, a redness, a pain, a swelling, a blistering, or a scaling. In some embodiments, the method comprises administering an amount sufficient to suppress virulence of S. aureus as measured by a reduction in expression of at least one of the following genes: gmk, agr, psma, saeR, ccpA, and SigB in S. aureus. In some embodiments, the reduction in expression is measured by a fluorescence reporter assay. In some embodiments, the composition is for use in the treatment of a skin condition. In some embodiments, the composition is for use in the treatment of inflammation.

In some embodiments, a composition comprises a bacterial species that is purified. In some embodiments, the bacterial species comprises Bacillus tropicus. In some embodiments, (a) the bacterial species is lyophilized; or (b) the composition is formulated for delivery to a skin. In some embodiments, the composition is formulated as: a suspension, an emulsion, a cream, a lotion, a tincture, a gel, a foam, a powder, an ointment, a paste, or an oil. In some embodiments, the composition can further comprise a second bacterial species. In some embodiments, the composition comprises at least 10\hat{ }3 colony forming units (cfu) per gram of bacteria. In some embodiments, the composition comprises 10\hat{ }3 to 10\hat{ }12 colony forming units (cfu) per gram of bacteria. In some embodiments, the bacterial species is grown in aerobic conditions. In some embodiments, the bacterial species is grown without animal products. In some embodiments, the bacterial species is grown in Tryptic Soy Broth (TSB). In some embodiments, the composition when stored in a sealed container placed at 20° C. can retain greater than about 10\hat{ }4 cfu after 6 months, as measured by cfu counts. In some embodiments, the composition can further comprise an excipient. In some embodiments, the composition can further comprise a lyoprotectant. In some embodiments, the composition can further comprise an emollient. In some embodiments, the composition can further comprise thiamine or a salt thereof. In some embodiments, the bacterial species when contacted with S. aureus can cause a reduction in expression of at least one of the following genes: gmk, agr, psma, saeR, ccpA, and SigB in S. aureus. In some embodiments, the reduction in expression is measured by a fluorescence reporter assay. In some embodiments, the bacterial species is present in an amount effective to suppress virulence of S. aureus as measured by a reduction in expression of at least one of the following genes: gmk, agr, psma, saeR, ccpA, and SigB in S. aureus. In some embodiments, the reduction in expression is measured by a fluorescence reporter assay. Also disclosed herein are methods of administering the composition. In some embodiments, the method comprises administering an amount sufficient to treat a disease selected from the group consisting of: atopic dermatitis, seborrheic dermatitis, inflammation, eczema, psoriasis, rosacea, mycoses, dermatophytosis, folliculitis, acne, alopecia, vitiligo, dandruff, chronic wound, skin ulcer, Netherton syndrome, hidradenitis suppurativa, sycosis vulgaris, staphylococcal scalded skin syndrome, impetigo, ecthyma, cellulitis, carbuncle, furuncle, and abscess. In some embodiments, the method comprises administering an amount sufficient to treat a condition selected from the group consisting of: pruritis, an aesthetic condition, and body odor. In some embodiments, the aesthetic condition comprises wrinkles or appearance of aging. In some embodiments, the method comprises administering an amount sufficient to reduce symptoms associated with atopic dermatitis. In some embodiments, the method comprises administering the composition to a subject who has an eczema prone microbiome prior to the administration. In some embodiments, the method comprises administering an amount sufficient to reduce symptoms associated with dry skin. In some embodiments, the method comprises administering an amount sufficient for a reduction of incidence of a condition associated with inflammation and wherein the condition associated with inflammation comprises an itch, a rash, a redness, a pain, a swelling, a blistering, or a scaling. In some embodiments, the method comprises administering an amount sufficient to suppress virulence of S. aureus as measured by a reduction in expression of at least one of the following genes: gmk, agr, psma, saeR, ccpA, and SigB in S. aureus. In some embodiments, the reduction in expression is measured by a fluorescence reporter assay. In some embodiments, the composition is for use in the treatment of a skin condition. In some embodiments, the composition is for use in the treatment of inflammation.

Also disclosed herein are methods for the treatment of inflammation, comprising topically administering a composition comprising Bacillus wiedmannii to a subject in need thereof. In some embodiments, the Bacillus wiedmannii is purified. In some embodiments, the Bacillus wiedmannii is present in an amount sufficient for treatment of inflammation.

Also disclosed herein are methods for reducing growth of Staphylococcus aureus on skin of a subject in need thereof, comprising topically administering a pharmaceutical composition comprising Bacillus velezensis, Bacillus wiedmannii, and Lysinibacillus macroides to a skin of the subject in need thereof. In some embodiments, the Bacillus velezensis, the Bacillus wiedmannii, and the Lysinibacillus macroides are purified. In some embodiments, the Bacillus velezensis, the Bacillus wiedmannii, and the Lysinibacillus macroides are viable, and present in an amount sufficient for reduction of S. aureus on a skin of the subject in need thereof.

Also disclosed herein are methods for reducing of symptoms associated with atopic dermatitis comprising topically administering a composition comprising Bacillus amyloliquefaciens to a subject in need thereof. In some embodiments, the Bacillus amyloliquefaciens is purified. In some embodiments, the Bacillus amyloliquefaciens is present in an amount sufficient for the reduction of symptoms associated with atopic dermatitis.

Also disclosed herein are methods for reducing an incidence of a condition associated with inflammation comprising topically administering a composition comprising Bacillus wiedmannii and Staphylococcus caprae to a subject in need thereof. In some embodiments, the Bacillus wiedmannii and the Staphylococcus caprae are purified. In some embodiments, the Bacillus wiedmannii and the Staphylococcus caprae are present in an amount sufficient for a reduction of the incidence of a condition associated with inflammation. In some embodiments, the condition associated with inflammation comprises an itch, a rash, a redness, a pain, a swelling, a blistering, or a scaling.

### EXAMPLES

### Example 1: Bacterial Strains and Screening

1750 bacterial isolates were picked from different sites on the skin of 20 healthy donors. Of the 1750 strains, 609 strains were frozen, and 180 strains were selected for an initial screen. The 180 strains were identified by 16S full length sequencing using Sanger sequencing. The reads were trimmed and concatenated. The 16S sequence was compared by a BLAST algorithm to the NCBI 16S database and species were identified as the top hit from the database. A minimal threshold was applied to the sequence for quality control. The top 28 strains from the 180 strains in the initial screen were further selected for several experiments. Table 1 shows the species from the top 28 strains selected.

Strains were screened in an array assay as a comprehensive approach to determine isolate(s) interaction with strains of S. aureus. Briefly, isolates and S. aureus strains were added to an array assay that contains thousands of microwells. Strains were mixed with molecules of different wavelengths for identification and when mixed create a unique wavelength code that is read by an imaging device to determine input. The S. aureus strains were promoter/reporter strains that were used to screen for inhibition of gene expression. Decreased expression of the promoter/reporter strains when grown with skin isolate(s) was indicated as a decreased fluoresce as compared to the promoter/reporter strain grown alone.

The S. aureus promoter/reporter strains contained a fluorescent reporter under the control of a promoter of a gene of interest. The promoter/reporter strains generated were as follows: Strain 1—agr promoter for quorum sensing induction, Strain 2—psmA promoter for a toxin that damages host tissue, Strain 3—saeR promoter for virulence regulation, Strain 4—sigB promoter for the stress response sigma factor, Strain 5—ccpA promoter for metabolism (e.g., carbon catabolite repression), and strain 6—GMK promoter for constitutive metabolic function.

Besides an array assay, strains could be tested for gene expression inhibition in a wide variety of assays such as in a microtiter plate growth assay, or another liquid culture growth assay. In another example, strains could be screened for gene inhibition on an agar plate.

### Example 2: Pairwise Assays of Bacterial Isolates for S. aureus Gene Expression Inhibition

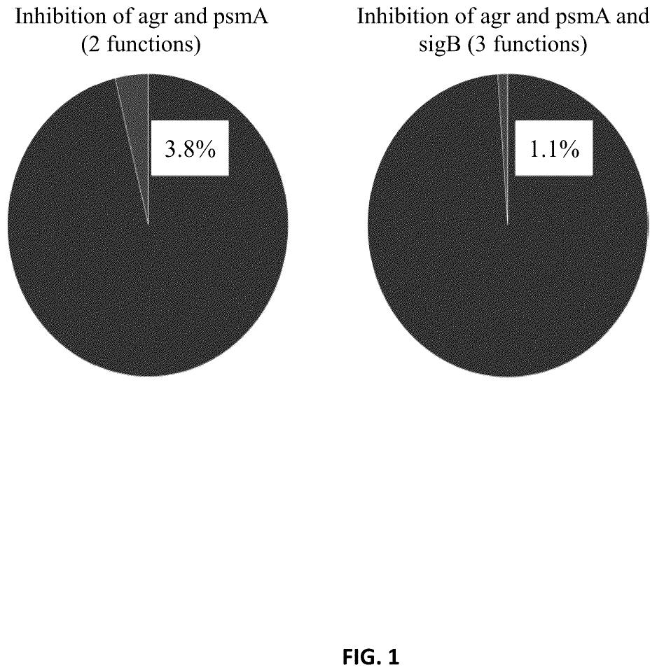

180 diverse bacterial isolates from the skin of healthy individuals were screened in a pairwise combinatorial screening assay against 4 S. aureus behavior reporters. S. aureus behaviors were measured via a set of plasmid-mediated “promoter-reporter” strains, whose fluorescence report on one specific activity. Combinations of strains were screened in an array assay for 10 days. The controls were: 1) no-isolate, which was a S. aureus monoculture, 2) wild-type S. aureus, and 3) streptomycin for a negative control, for S. aureus eradication. The screen analyzed the reporters GMK for constitutive metabolic function, agr for quorum sensing induction, psmA for toxin that damages host tissue, and sigB for the stress response sigma factor. Referring to FIG. 1, FIG. 1 on the left shows out of the 180 bacterial isolates that were screened only 3.8% of the pairwise combinations inhibited agr and psmA to 3 folds or 12.5% of monoculture levels. Similarly, FIG. 1 on the right shows out of the 180 bacterial isolates that were screened, only 1.1% of the pairwise combinations inhibited agr, psmA, and sigB to 3 folds or 12.5% of monoculture levels. These data show combinations that possess multiple functions are rare.

### Example 3: Three-Wise Assays of Bacterial Isolates for S. aureus Gene Expression Inhibition

Bacterial isolates from the skin of healthy individuals were screened in a three-wise combinatorial screening assay against 6 S. aureus behavior reporters. S. aureus behaviors were measured via a set of plasmid-mediated “promoter-reporter” strains, whose fluorescence report on one specific activity. Combinations of strains were screened in an array assay for 4 days. The strain mixture conditions were: 1) no-isolate, which was a S. aureus monoculture, 2) wild-type S. aureus with isolated strains, and 3) streptomycin for a negative control, for S. aureus eradication. The screen analyzed the expression of the reporters agr for quorum sensing induction, psmA for toxin that damages host tissue, saeR for virulence regulation, sigB for the stress response sigma factor, ccpA for metabolism (e.g., carbon catabolite repression), and GMK for constitutive metabolic function.

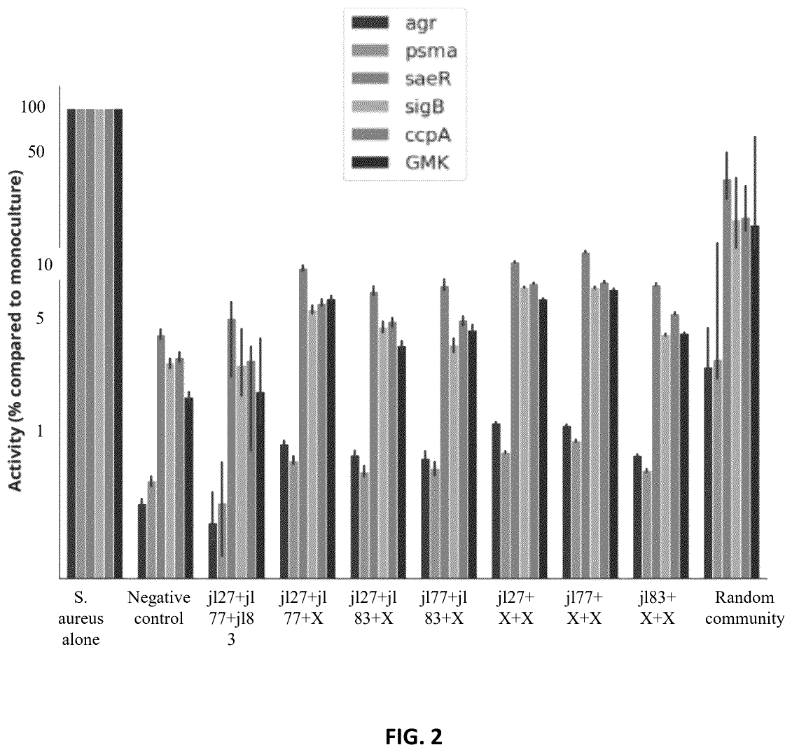

Referring to FIG. 2, the Y-axis shows the percent activity as compared to the S. aureus monoculture. The X-axis shows the strains in the combinatorial screening assay. “X” with one or more strains indicates all individual strains from the top 28 isolates were added to the mixture and the average is shown from the mixtures. For example, the jl27+jl77+X data is an average of 26 different 3-wise compositions. In another example, the jl27+X+X is an average of 351different compositions. The random community is a 3 isolate community randomly selected from the top 28 isolates. The reporters are shown from left to right and are as follows: agr, psmA, saeR, sigB, ccpA and GMK. Three-wise combinations containing Strain jl.83, Strain jl.27 and Strain jl.77 were effective at suppressing S. aureus agr (quorum sensing) and psmA (toxin production); pairs of these were stronger still; and all three together produced the strongest effect. In some conditions, three-wise combinations containing Strain jl.83, Strain jl.27 and Strain jl.77 were effective at suppressing S. aureus saeR, sigB, ccpA and GMK. All three together decreased activity of all reporters to less than 10% activity compared to monoculture. The decrease was unexpected as compared to a random community which only decreased expression levels to about 20% to 40% in saeR, sigB, ccpA and GMK as compared to the monoculture. The data shows strain combinations can be effective at inhibiting S. aureus gene expression.

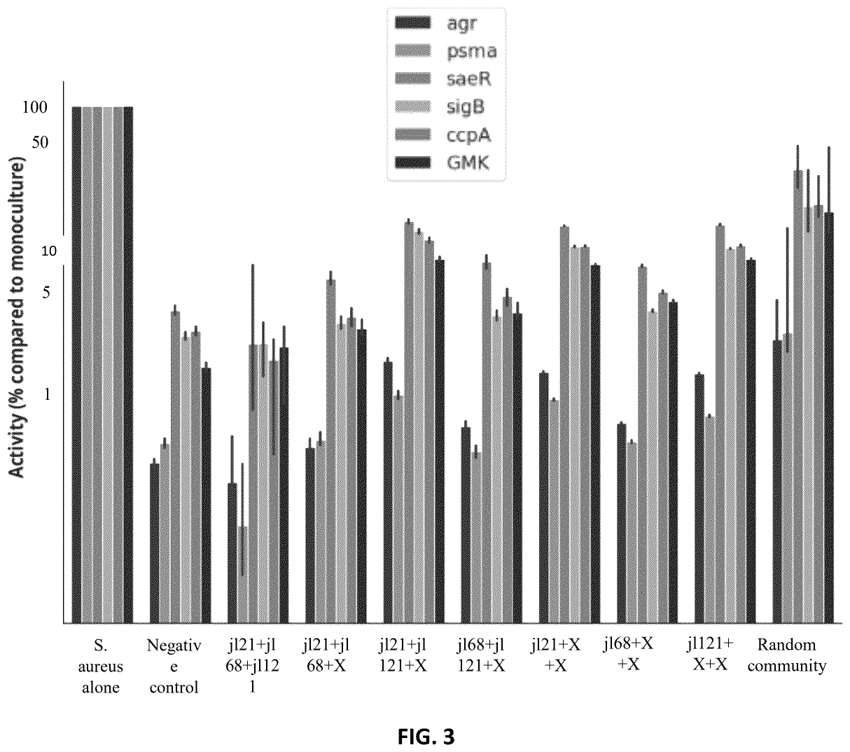

Referring to FIG. 3, the Y-axis shows the percent activity as compared to the S. aureus monoculture. The X-axis shows the strains in the combinatorial screening assay. “X” with one or more strains indicates all individual strains from the top 28 isolates were added to the mixture and the average is shown from the mixtures. For example, the jl21+jl68+X data is an average of 26 different 3-wise compositions. In another example, the jl21+X+X is an average of 351 different compositions. The random community is a 3 isolate community randomly selected from the top 28 isolates. The reporters are shown from left to right and are as follows: agr, psmA, saeR, sigB, ccpA and GMK. Three-wise combinations containing Strain jl.21, Strain jl.68, and Strain jl.121 were effective at suppressing S. aureus agr (quorum sensing) and psmA (toxin production); pairs of these were stronger still; and all three together produced the strongest effect. In some conditions, three-wise combinations containing Strain jl.21, Strain jl.68, and Strain jl.121 were effective at suppressing S. aureus saeR, sigB, ccpA and GMK. All three together decreased activity of all reporters to less than 10% activity compared to monoculture. The decrease was unexpected as compared to a random community which only decreased expression levels to about 20% to 40% in saeR, sigB, ccpA and GMK as compared to the monoculture. The data shows strain combinations can be effective at inhibiting S. aureus gene expression.

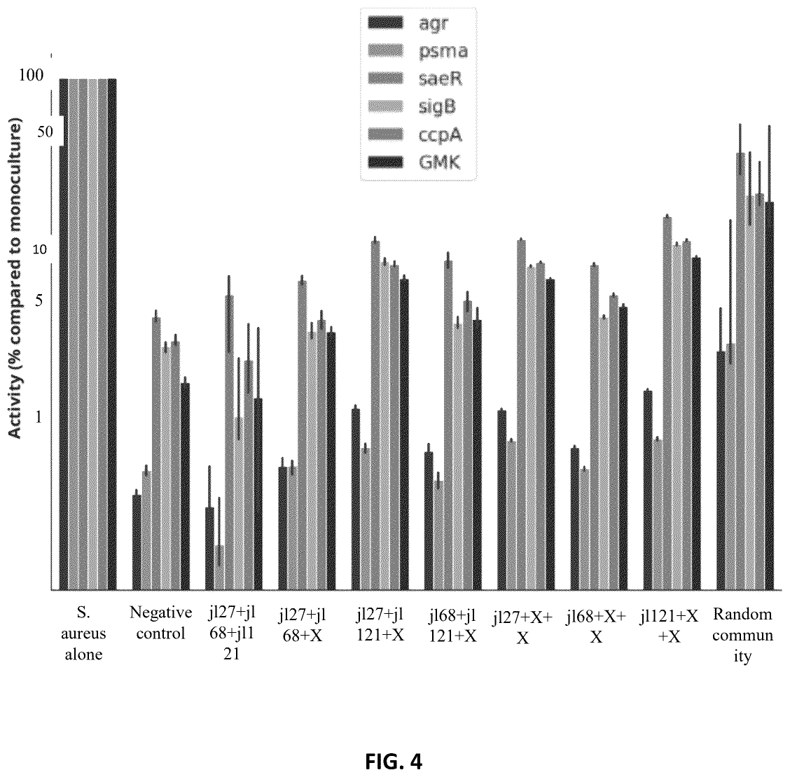

Referring to FIG. 4, the Y-axis shows the percent activity as compared to the S. aureus monoculture. The X-axis shows the strains in the combinatorial screening assay. “X” with one or more strains indicates all individual strains from the top 28 isolates were added to the mixture and the average is shown from the mixtures. For example, the jl27+jl68+X data is an average of 26 different 3-wise compositions. In another example, the jl27+X+X is an average of 351 different compositions. The random community is a 3 isolate community randomly selected from the top 28 isolates. The reporters are shown from left to right and are as follows: agr, psmA, saeR, sigB, ccpA and GMK. Three-wise combinations containing Strain jl.27, Strain jl.68, and Strain jl.121 were effective at suppressing S. aureus agr (quorum sensing) and psmA (toxin production); pairs of these were stronger still; and all three together produced the strongest effect. In some conditions, three-wise combinations containing Strain jl.27, Strain jl.68, and Strain jl.121 were effective at suppressing S. aureus saeR, sigB, ccpA and GMK. All three together decreased activity of all reporters to less than 10% activity compared to monoculture. The decrease was unexpected as compared to a random community which only decreased expression levels to about 20% to 40% in saeR, sigB, ccpA and GMK as compared to the monoculture. The data shows strain combinations can be effective at inhibiting S. aureus gene expression.

### Example 4: Assays of S. aureus Gene Expression Inhibition in Different Environmental Conditions

Three wise combinations of Strain jl.83, Strain jl.27 Strain jl.77 Strain jl.21, Strain jl.68,and Strain jl.121 were screened in an assessment of the behavior of S. aureus when an additional environmental component such as a nutrient was added to the mixture. S. aureus behaviors were measured via a set of plasmid-mediated “promoter-reporter” strains, whose fluorescence report on one specific activity. Combinations of strains and environmental conditions were screened in an array for 1 day. The strain mixture conditions were: 1) a S. aureus monoculture, and 2) S. aureus mixed with three skin isolates. The screen analyzed the expression of the reporters agr for quorum sensing induction, psmA for toxin that damages host tissue, and GMK for constitutive metabolic function.

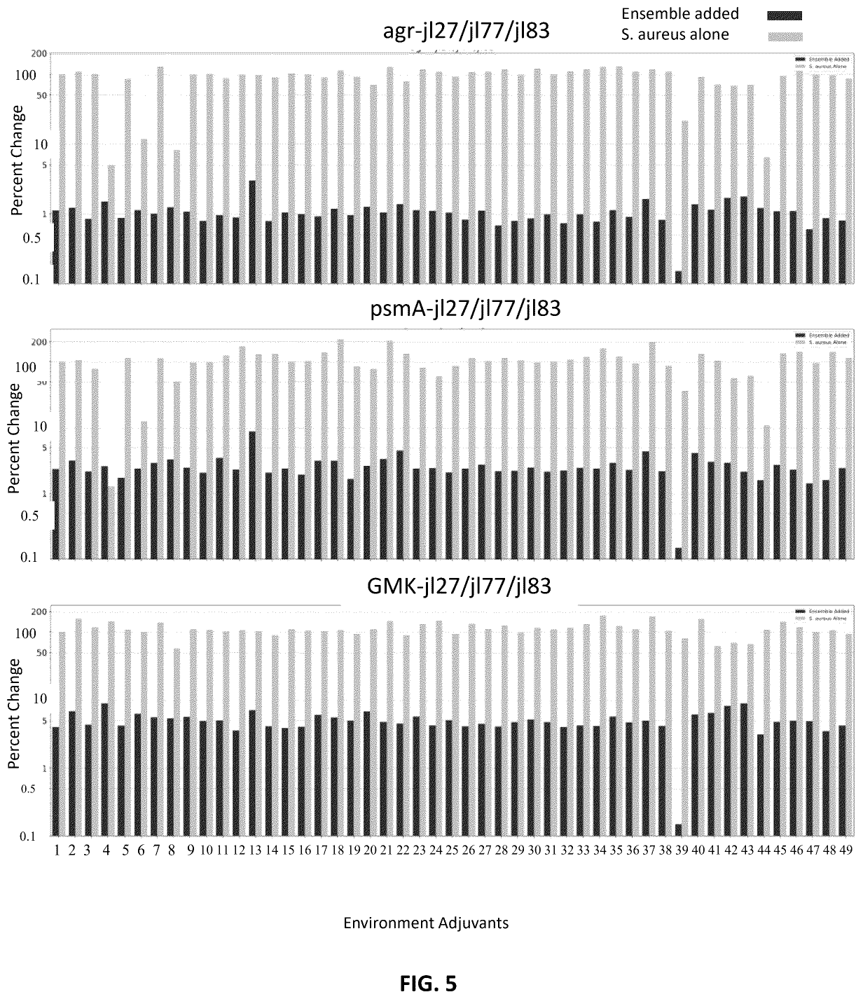

Referring to FIG. 5, the Y-axis shows the percent change as compared to the S. aureus monoculture. The monoculture conditions are shown with the gray bars and the black bars show the conditions in which Strain jl.83, Strain jl.27 and Strain jl.77 were added to the culture. The X-axis shows the environmental condition that was tested. The conditions are from left to right (1) base medium, then base medium supplemented with one of the following: (2) acetate, (3) beta-alanine, (4) bicarbonate, (5) biotin, (6) butyrate, (7) caffeine, (8) citrate, (9) creatine, (10) D-cellobiose, (11) D-fructose, (12) D-glucosamine, (13) D-glucose, (14) D-mannitol, (15) D-raffinose, (16) D-sorbitol, (17) D-sucrose, (18) D-trehalose, (19) D-xylose, (20) formate, (21) GlcNAc, (22) glycerol, (23) glycine, (24) L-alanine, (25) L-arabinose, (26) L-arginine, (27) L-citrulline, (28) L-glutamine, (29) L-hydroxyproline, (30) L-isoleucine, (31) L-leucine, (32) L-methionine, (33) L-ornithine, (34) L-proline, (35) L-serine, (36) L-taurine, (37) L-threonine, (38) L-valine, (39) L-ascorbate, (40) L-lactate, (41) nicotinamine, (42) polysorbate 20, (43) polysorbate 80, (44) propionate, (45) pyruvate, (46) succinate, (47) thiamine, (48) triethanolamine, or (49) urea. The data shows the strain combination containing Strain jl.83, Strain jl.27 and Strain jl.77 were effective at suppressing S. aureus agr (quorum sensing), psmA (toxin production), and GMK (metabolic function) in all tested environmental conditions, including conditions that aided S. aureus growth.

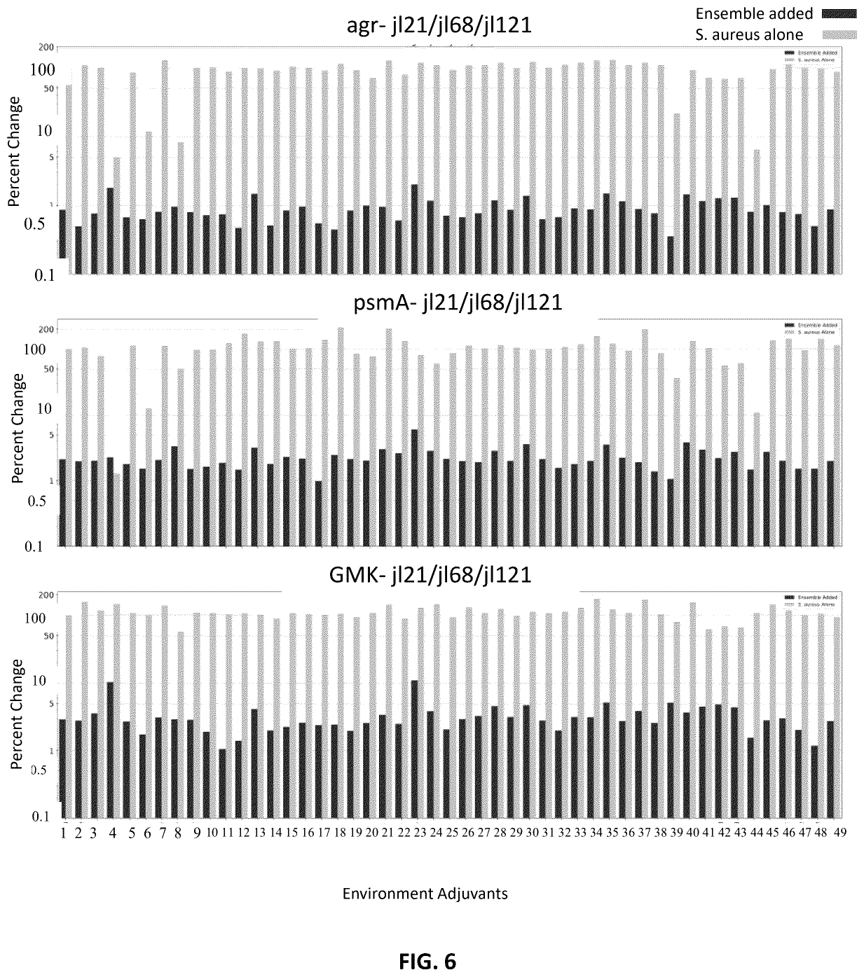

Referring to FIG. 6, the Y-axis shows the percent change as compared to the S. aureus monoculture. The monoculture conditions are shown with the gray bars and the black bars show the conditions in which Strain jl.21, Strain jl.68, and Strain jl.121 were added to the culture. The X-axis shows the environmental condition that was tested. The conditions are from left to right (1) base medium, then base medium supplemented with one of the following: (2) acetate, (3) beta-alanine, (4) bicarbonate, (5) biotin, (6) butyrate, (7) caffeine, (8) citrate, (9) creatine, (10) D-cellobiose, (11) D-fructose, (12) D-glucosamine, (13) D-glucose, (14) D-mannitol, (15) D-raffinose, (16) D-sorbitol, (17) D-sucrose, (18) D-trehalose, (19) D-xylose, (20) formate, (21) GlcNAc, (22) glycerol, (23) glycine, (24) L-alanine, (25) L-arabinose, (26) L-arginine, (27) L-citrulline, (28) L-glutamine, (29) L-hydroxyproline, (30) L-isoleucine, (31) L-leucine, (32) L-methionine, (33) L-ornithine, (34) L-proline, (35) L-serine, (36) L-taurine, (37) L-threonine, (38) L-valine, (39) L-ascorbate, (40) L-lactate, (41) nicotinamine, (42) polysorbate 20, (43) polysorbate 80, (44) propionate, (45) pyruvate, (46) succinate, (47) thiamine, (48) triethanolamine, or (49) urea. In all cases, the strain combination was able to decrease expression of all three reporters to less than 10% monoculture levels. The data shows the strain combination containing Strain jl.21, Strain jl.68, and Strain jl.121 were effective at suppressing S. aureus agr (quorum sensing), psmA (toxin production), and GMK (metabolic function) in all tested environmental conditions, including conditions that aided S. aureus growth. In all cases, the strain combination was able to decrease expression of all three reporters to less than 10% monoculture levels.

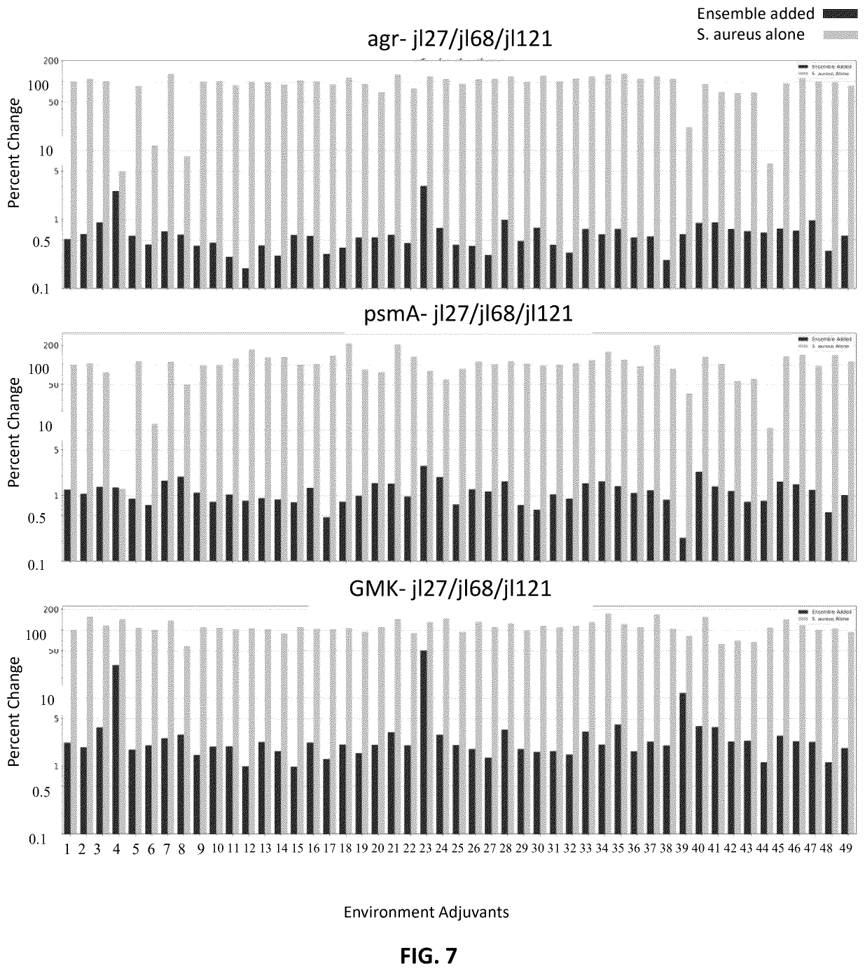

Referring to FIG. 7, the Y-axis shows the percent change as compared to the S. aureus monoculture. The monoculture conditions are shown with the gray bars and the black bars show the conditions in which Strain jl.27, Strain jl.68, and Strain jl.121 were added to the culture. The X-axis shows the environmental condition that was tested. The conditions are from left to right (1) base medium, then base medium supplemented with one of the following: (2) acetate, (3) beta-alanine, (4) bicarbonate, (5) biotin, (6) butyrate, (7) caffeine, (8) citrate, (9) creatine, (10) D-cellobiose, (11) D-fructose, (12) D-glucosamine, (13) D-glucose, (14) D-mannitol, (15) D-raffinose, (16) D-sorbitol, (17) D-sucrose, (18) D-trehalose, (19) D-xylose, (20) formate, (21) GlcNAc, (22) glycerol, (23) glycine, (24) L-alanine, (25) L-arabinose, (26) L-arginine, (27) L-citrulline, (28) L-glutamine, (29) L-hydroxyproline, (30) L-isoleucine, (31) L-leucine, (32) L-methionine, (33) L-ornithine, (34) L-proline, (35) L-serine, (36) L-taurine, (37) L-threonine, (38) L-valine, (39) L-ascorbate, (40) L-lactate, (41) nicotinamine, (42) polysorbate 20, (43) polysorbate 80, (44) propionate, (45) pyruvate, (46) succinate, (47) thiamine, (48) triethanolamine, or (49) urea. In all cases, the strain combination was able to decrease expression of all three reporters to less than about 50% monoculture levels. The data shows the strain combination containing Strain jl.27, Strain jl.68, and Strain jl.121 were effective at suppressing S. aureus agr (quorum sensing), psmA (toxin production), and GMK (metabolic function) in all tested environmental conditions, including conditions that aided S. aureus growth.

### Example 5: Assay of Thiamine Enhancement of S. aureus Gene Expression Inhibition

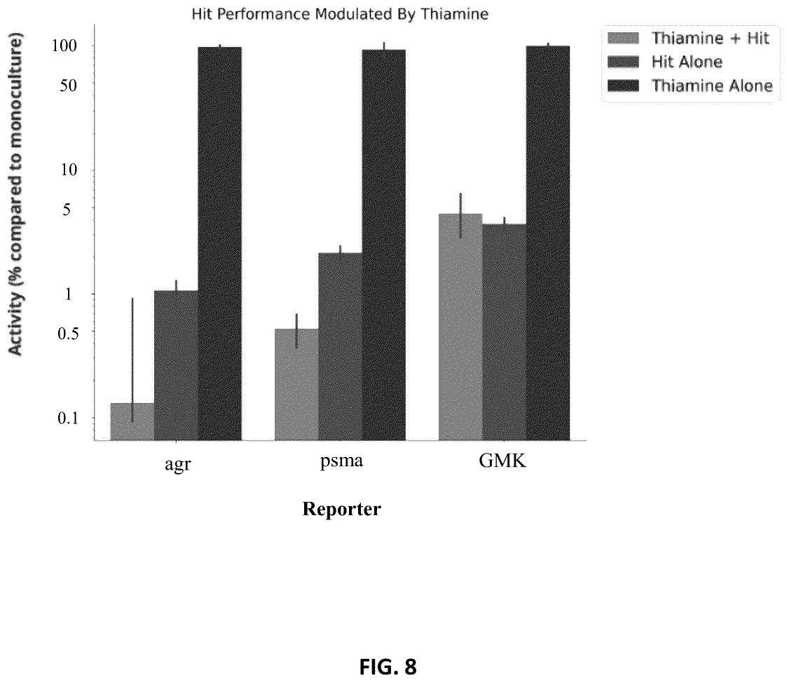

Strain jl.83, Strain jl.27 and Strain jl.77 were screened in an assessment of the behavior of S. aureus when thiamine was added to the mixture. S. aureus behaviors were measured via a set of plasmid-mediated “promoter-reporter” strains, whose fluorescence report on one specific activity. Combinations of strains and environmental conditions were screened in an array assay for 1 day. The strain mixture conditions were: (1) a S. aureus monoculture, (2) S. aureus monoculture in medium with excess thiamine, (3) S. aureus mixed with Strain jl.83, Strain jl.27 and Strain jl.77 and (4) S. aureus mixed with Strain jl.83, Strain jl.27 and Strain jl.77 in medium with excess thiamine. The array assay analyzed the expression of the reporters agr for quorum sensing induction, psmA for toxin that damages host tissue, and GMK for constitutive metabolic function. Referring to FIG. 8, the Y-axis shows the percent activity as compared to the S. aureus monoculture. The X-axis shows the expression of S. aureus agr (quorum sensing), psmA (toxin production), and GMK (metabolic function). For each gene, three strain mixture conditions are shown from left to right the conditions are: condition (4), condition (3), and condition (2). The addition of thymine surprisingly showed a synergistic effect when added with Strain jl.83, Strain jl.27 and Strain jl.77 in suppressing the expression of S. aureus agr (quorum sensing), and psmA (toxin production). For example, agr expression and psmA expression was decreased greater than 2-fold in both cases. This data shows thymine displayed a synergistic effect with the mixture of bacteria to increase S. aureus gene inhibition.

### Example 6: Isolated Strains Growth in Animal-Free Media

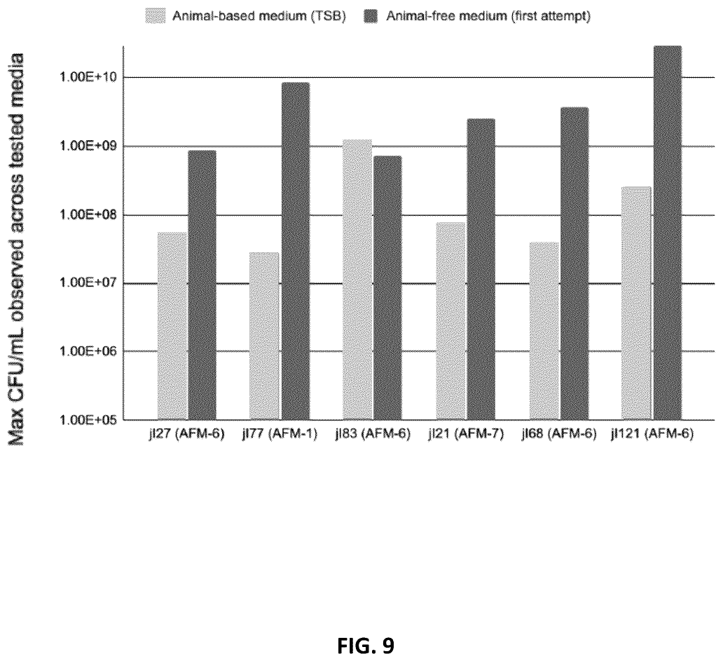

Bacterial isolates Strain jl.27, Strain jl.77, Strain jl.83, Strain jl.21, Strain jl.68, and Strain jl.121 were grown in comparable conditions in an animal based medium tryptic soy broth (TSB) and in various animal-free media (AFM). Strains were grown for 48 hours after inoculation with the same number of bacteria. Referring to FIG. 9, the Y-axis shows the colony-forming unit (cfu)/mL observed across the tested media. The X-axis indicates the strain tested and the animal-free media formulation used for the strain tested (e.g., AFM-6 for animal-free media formulation 6). The strains tested were Strain jl.27 (jl27), Strain jl.77 (jl77), Strain jl.83 (jl83), Strain jl.21 (jl21), Strain jl.68 (jl68), and Strain jl.121 (jl121). For most of the strains grown in animal-free media there was a 10-100× improvement in cultivation cfu/ml after 48 hours. The data shows the strains can be cultivated in a variety of media and can be manufactured in animal free media for production of a composition, such as a pharmaceutical composition.

### Example 7: Assays of S. aureus Growth Inhibition with Combinations of Bacterial Isolates

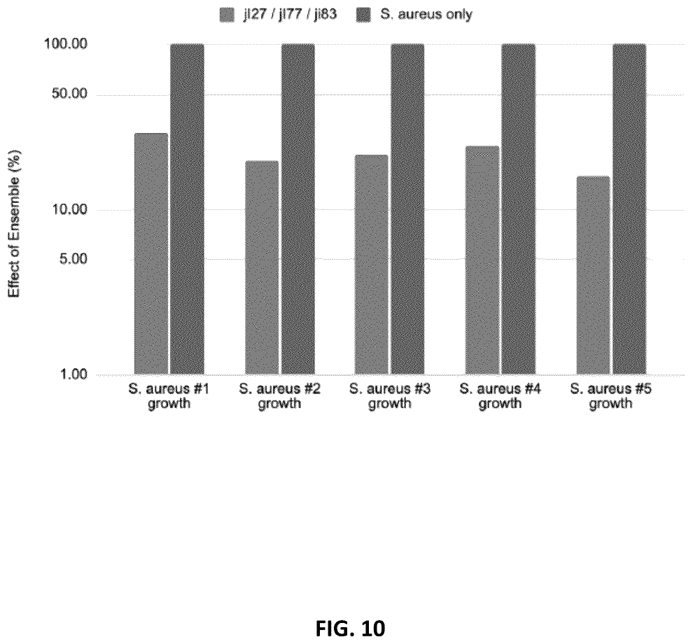

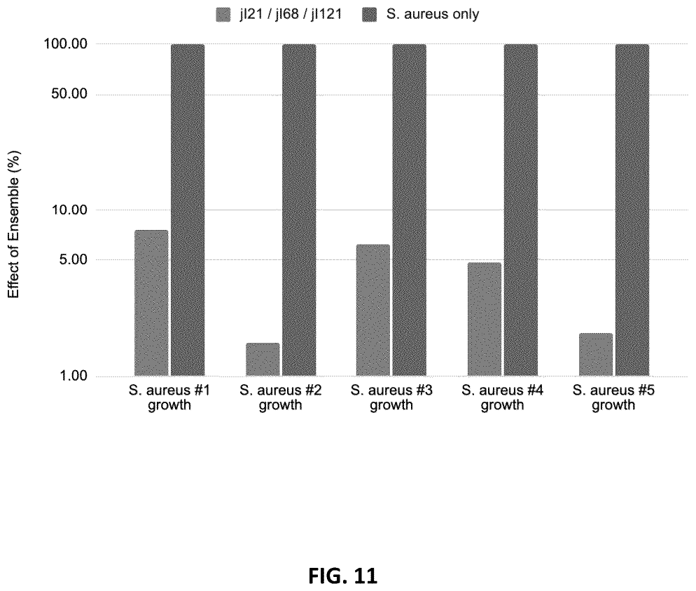

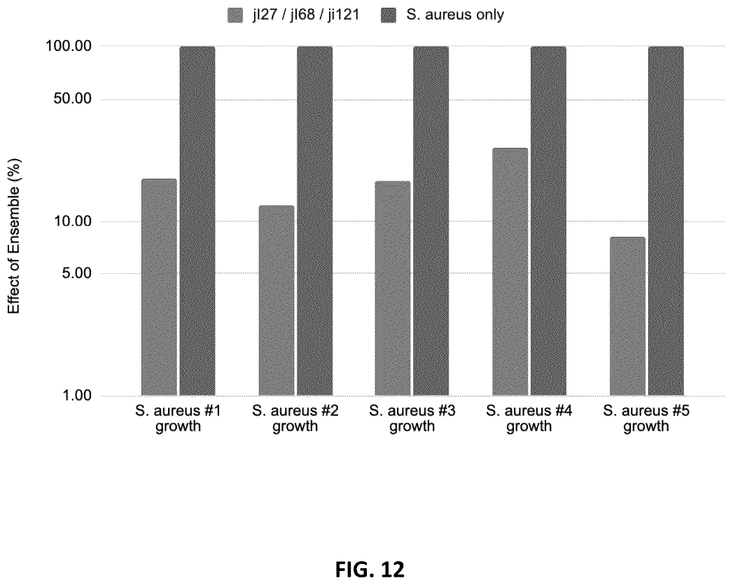

Bacterial isolates from the skin of healthy individuals were tested for their ability to inhibit the growth of several S. aureus strains. Strains were grown for 48 hours after inoculation with the same number of bacteria. Referring to FIG. 10, FIG. 11 and FIG. 12, the Y-axis shows the effect of growth inhibition by the strain combination. The X-axis shows the growth of different S. aureus strains in the presence of an ensemble (e.g., the three-strain combination) or the S. aureus strain alone. For the S. aureus strain tested, the ensemble is the left bar graph and the S. aureus strain alone is the right bar graph. FIG. 10 tested the ensemble jl27/jl77/jl83 (Strain jl.27, Strain jl.77 and Strain jl.83), FIG. 11 tested the ensemble jl21/jl68/jl121 (Strain jl.21, Strain jl.68, and Strain jl.121), FIG. 12 tested the ensemble jl27/jl68/jl121 (Strain jl.27,Strain jl.68, and Strain jl.121). All three tested strain combinations were able to inhibit growth of different S. aureus strains. This suggests the strain combinations are effective against a wide variety of S. aureus strains.

### Example 8: Assays of S. aureus Gene Expression Inhibition with Bacillus wiedmannii Alone and in Combination with Additional Strains

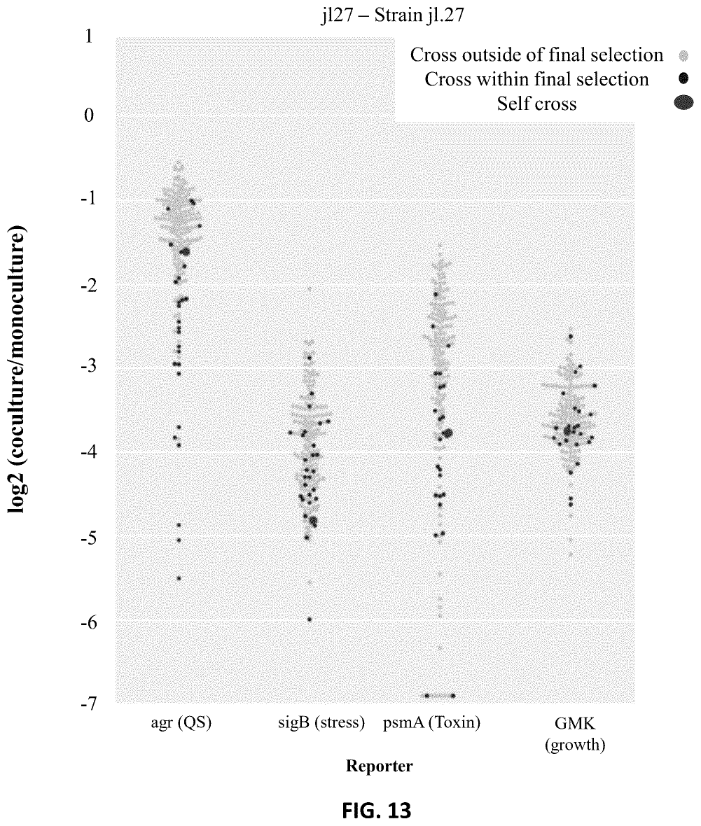

Strain jl.27 was isolated from the skin of a healthy individual and was screened in a pair-wise combinatorial screening assay against 4 S. aureus behavior reporters. S. aureus behaviors were measured via a set of plasmid-mediated “promoter-reporter” S. aureus strains, whose fluorescence report on one specific activity. Combinations of strains were screened in an array assay. The strain mixture conditions were: 1) Strain jl.27 self-cross, 2) Strain jl.27 with random skin isolate, and 3) Strain jl.27 with another isolate that showed inhibition of S. aureus gene expression. The screen analyzed the expression of the reporters agr for quorum sensing induction, sigB for the stress response sigma factor, psmA for toxin that damages host tissue, and GMK for constitutive metabolic function. Referring to FIG. 13, the Y-axis shows the log₂ expression change of the coculture/a S. aureus promoter-reporter monoculture. The X-axis shows the gene promoters tested. The big circles indicate the data points for the self-cross (Strain jl.27 and Strain jl.27). The small black circles indicate Strain jl.27 crossed with another isolate that showed inhibition of S. aureus gene expression. The small light gray circles indicate Strain jl.27 with random skin isolate. The data shows Strain jl.27 had a strong suppressive effect on growth, stress, and toxin production of S. aureus. It also appeared in many combinations where quorum sensing was strongly suppressed in S. aureus. This data shows Strain jl.27 is effective alone or in combination at inhibiting S. aureus gene expression.

### Example 9: Inhibition of S. aureus Gene Expression in the Presence of Additional Microbes

Three wise combination, two wise combinations and individual strains of Strain jl.83, Strain jl.27 Strain jl.77 Strain jl.21, Strain jl.68, and Strain jl.121 were screened in an assessment of the behavior of S. aureus when in the presence of different microbes. The bacterial strains added to the mixture were randomly selected from 92 strains selected from the original 609 frozen strain collection. S. aureus behaviors were measured via a set of plasmid-mediated “promoter-reporter” strains, whose fluorescence report on one specific activity. The screen analyzed the expression of the reporters: agr for quorum sensing induction, psmA for toxin that damages host tissue, and GMK for constitutive metabolic function. Combinations of strains were screened in an array after 1 day of growth. The strain mixture conditions were: 1) three-wise combination with an additional set of 4 strains, 2) two-wise combination with an additional set of 5 strains, and 3) one individual strain with an additional set up 6 strains. For example, jl121/jl21/jl68+4× shows tested communities where jl.121, jl.21 and jl.68 and up to 4 additional microbes (chosen among 89 strains) were present. In another example, jl121/jl21+5× shows tested communities where jl.121 and jl.21 and up to 5 additional microbes (chosen among 90 strains) were present. In another example, jl121+6× shows tested communities where jl.121 and up to 6 additional microbes (chosen among 91 strains) were present.

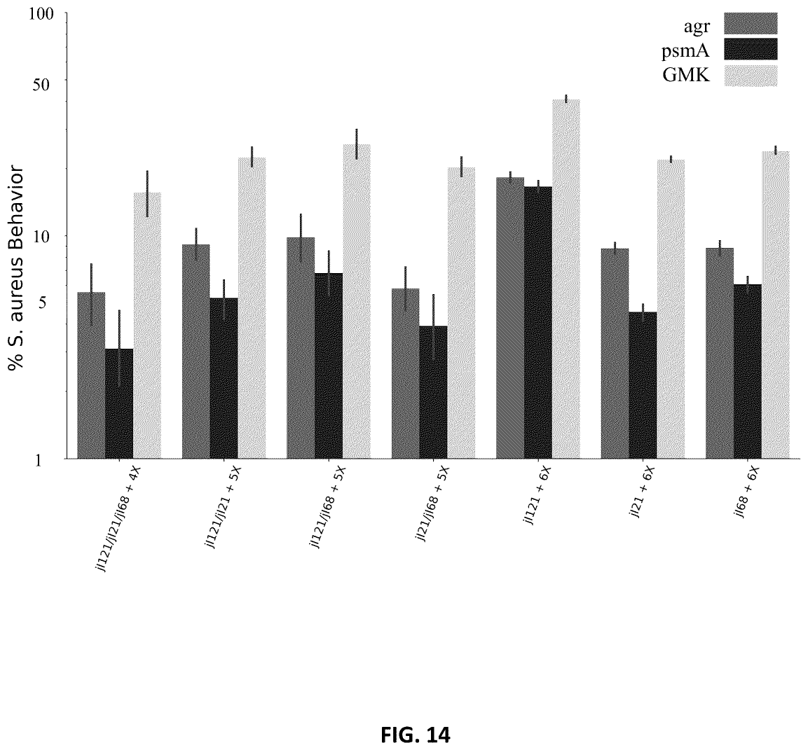

Referring to FIG. 14, the Y-axis shows the percent expression of the reporter as compared to the S. aureus monoculture (i.e., the monoculture is 100%). The X-axis shows the strain combinations that were tested. The tested strains were Strain jl.121, Strain jl.21, and Strain jl.68. These strains were tested in different combinations with a random selection of strains to form 7-strain communities. The data shows the strain combination containing Strain jl.121, Strain jl.21, and Strain jl.68 were (1) effective at suppressing S. aureus agr (quorum sensing), psmA (toxin production), and GMK (metabolic function); (2) strong performance of the strain combination (ensemble) still occurs even with other microbes present, suggesting the combination will perform well in a native microbiome; and (3) that performance is stronger than when any subset of the 3-species combination is present. For example, a 3-species combination of Strain jl.121, Strain jl.21, and Strain jl.68 are robust to the larger community and more robust than their subsets as agr expression was reduced to less than about 5%, psmA expression was reduced to less than about 2%, and GMK expression was reduced to less than about 20%.

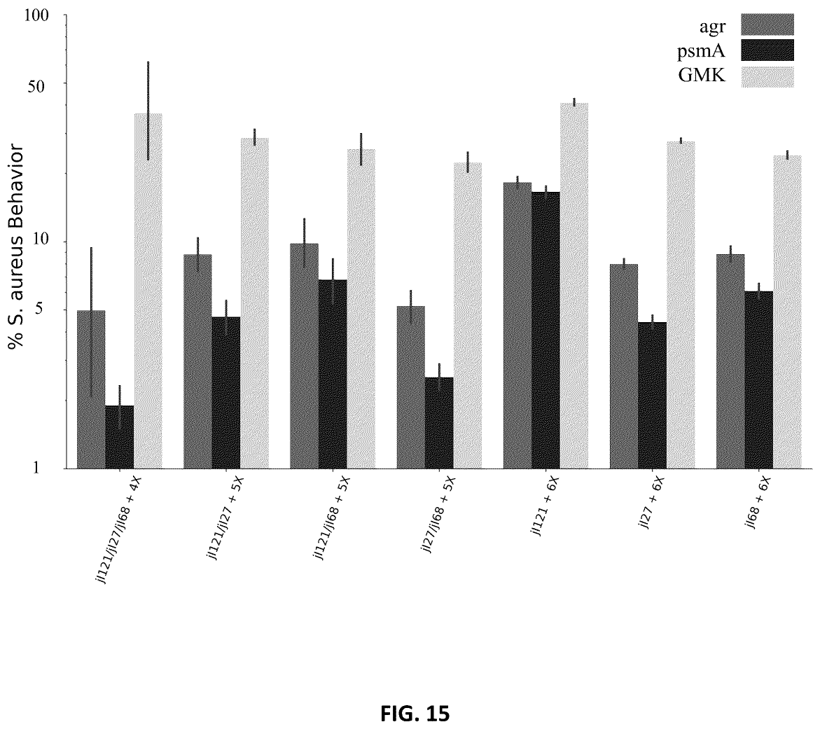

Referring to FIG. 15, shows the percent expression of the reporter as compared to the S. aureus monoculture (i.e., the monoculture is 100%). The X-axis shows the strain combinations that were tested. The tested strains were Strain jl.121, Strain jl.27, and Strain jl.68. These strains were tested in different combinations with a random selection of strains to form 7-strain communities. The data shows the strain combination containing Strain jl.121, Strain jl.27, and Strain jl.68 were (1) effective at suppressing S. aureus agr (quorum sensing), psmA (toxin production), and GMK (metabolic function); (2) strong performance of the strain combination (ensemble) still occurs even with other microbes are present, suggesting the combination will perform well in a native microbiome; and (3) that performance is stronger than when any subset of the 3-species combination is present. For example, a 3-species combination of Strain jl.121, Strain jl.27, and Strain jl.68 are robust to the larger community and more robust than their subsets as agr expression was reduced to less than about 5%, psmA expression was reduced to less than about 2%, and GMK expression was reduced to less than about 40%.

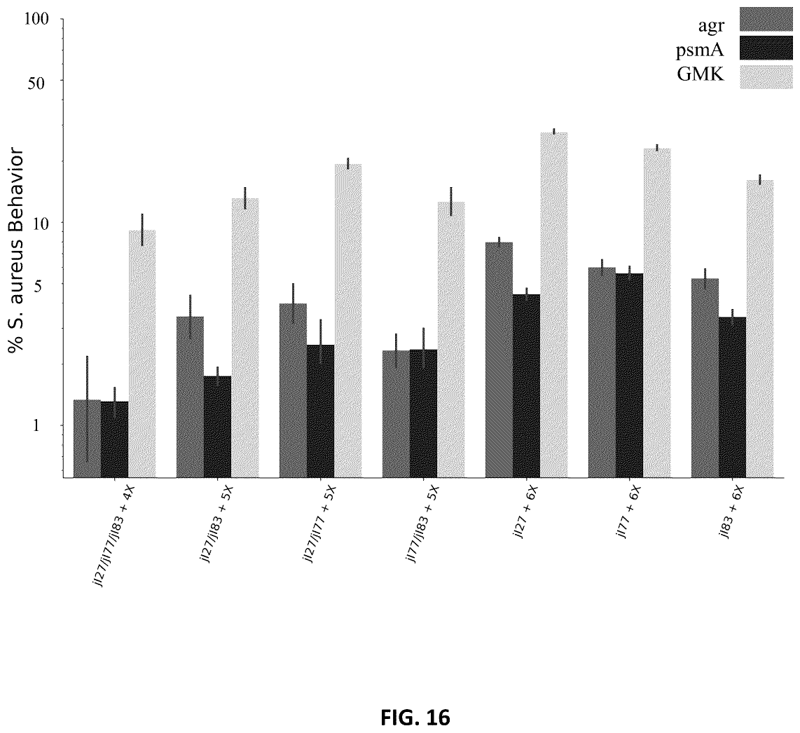

Referring to FIG. 16, shows the percent expression of the reporter as compared to the S. aureus monoculture (i.e., the monoculture is 100%). The X-axis shows the strain combinations that were tested. The tested strains were Strain jl.27, Strain jl.77, and Strain jl.83. These strains were tested in different combinations with a random selection of strains to form 7-strain communities. The data shows the strain combination containing Strain jl.27, Strain jl.77, and Strain jl.83 were (1) effective at suppressing S. aureus agr (quorum sensing), psmA (toxin production), and GMK (metabolic function); (2) strong performance of the strain combination (ensemble) still occurs even with other microbes are present, suggesting the combination will perform well in a native microbiome; and (3) that performance is stronger than when any subset of the 3-species combination is present. For example, a 3-species combination of Strain jl.27, Strain jl.77, and Strain jl.83 are robust to the larger community and more robust than their subsets as agr expression was reduced to less than about 2%, psmA expression was reduced to less than about 2%, and GMK expression was reduced to less than about 10%.

### Example 10: Clinical Study of Skin Inflammation

A human female subject diagnosed with inflammation of the skin is treated with a pharmaceutical composition comprising Strain jl.21, Strain jl.68, and Strain jl.121. After administration of the pharmaceutical composition for 2 weeks, the following is observed: an amount of pain, an amount of redness, an amount of swelling, and an amount of itching.

### Example 11: Clinical Study of Atopic Dermatitis

A human male subject diagnosed with atopic dermatitis is treated with a pharmaceutical composition comprising Strain jl.83, Strain jl.27 and Strain jl.77. After administration of the pharmaceutical composition for 3 weeks, the following is observed: an amount of flakiness, an amount of redness, an amount of dryness, and an amount of peeling.

### Example 12: Sequences of Isolated Strains

Isolated strains were subjected to whole genome sequencing and assembly of generated contigs. Additionally, 16s rRNA gene sequences were sequenced by Sanger sequencing and manually trimmed. The 16s rRNA sequences from the isolated strains are presented in Table 6. Additionally, several identifier sequences were pulled from the assembly for each isolated strain for identification. The identifier sequences from the isolated strains are presented in Table 7. To identify the identifier sequences, the genome assemblies were assessed for strain identifying sequences. The assemblies were preprocessed by: removing 1000 bp from each side of each assembly sequence to minimize edge effects and if the resulting sequence length after cropping was <10 kbp, it was dropped from consideration. A series of new sub-sequences was created by applying a sliding window of some length, with a 10% overlap across windows; a 5000 bp window with a 500 bp overlap was used and a 1200 bp window with a 120 bp overlap was used. The sequences were searched by BLAST from a nucleotide collection database (the Nucleotide Collection (nt) database) provided by The National Center for Biotechnology Information (NCBI) downloaded on Sep. 17, 2022. Identifier sequences that had BLAST sequences search results with greater than or equal to 99% query coverage and less than 98% sequence percent identity are included in Table 7.

TABLE 6
16s rRNA sequences of Isolated Strains

| Strain | |
|---|---|
| Name | 16s rRNA Sequence |
| Strain | TCTTATGAAGTTAGCGGCGGACGGGTGAGTAACACGTGGGTAACCTGCCCATAAGA |
| jl.27 | CTGGGATAACTCCGGGAAACCGGGGCTAATACCGGATAACATTTTGAACCGCATGG |
| SEQ ID | TTCGAAATTGAAAGGCGGCTTCGGCTGTCACTTATGGATGGACCCGCGTCGCATTA |
| NO: 1 | GCTAGTTGGTGAGGTAACGGCTCACCAAGGCAACGATGCGTAGCCGACCTGAGAG |
| | GGTGATCGGCCACACTGGGACTGAGACACGGCCCAGACTCCTACGGGAGGCAGCA |
| | GTAGGGAATCTTCCGCAATGGACGAAAGTCTGACGGAGCAACGCCGCGTGAGTGAT |
| | GAAGGCTTTCGGGTCGTAAAACTCTGTTGTTAGGGAAGAACAAGTGCTAGTTGAATA |
| | AGCTGGCACCTTGACGGTACCTAACCAGAAAGCCACGGCTAACTACGTGCCAGCAG |
| | CCGCGGTAATACGTAGGTGGCAAGCGTTATCCGGAATTATTGGGCGTAAAGCGCGC |
| | GCAGGTGGTTTCTTAAGTCTGATGTGAAAGCCCACGGCTCAACCGTGGAGGGTCAT |
| | TGGAAACTGGGAGACTTGAGTGCAGAAGAGGAAAGTGGAATTCCATGTGTAGCGGT |
| | GAAATGCGTAGAGATATGGAGGAACACCAGTGGCGAAGGCGACTTTCTGGTCTGTA |
| | ACTGACACTGAGGCGCGAAAGCGTGGGGAGCAAACAGGATTAGATACCCTGGTAGT |
| | CCACGCCGTAAACGATGAGTGCTAAGTGTTAGAGGGTTTCCGCCCTTTAGTGCTGA |
| | AGTTAACGCATTAAGCACTCCGCCTGGGGAGTACGGCCGCAAGGCTGAAACTCAAA |
| | GGAATTGACGGGGGCCCGCACAAGCGGTGGAGCATGTGGTTTAATTCGAAGCAAC |
| | GCGAAGAACCTTACCAGGTCTTGACATCCTCTGAAAACCCTAGAGATAGGGCTTCTC |
| | CTTCGGGAGCAGAGTGACAGGTGGTGCATGGTTGTCGTCAGCTCGTGTCGTGAGAT |
| | GTTGGGTTAAGTCCCGCAACGAGCGCAACCCTTGATCTTAGTTGCCATCATTAAGTT |
| | GGGCACTCTAAGGTGACTGCCGGTGACAAACCGGAGGAAGGTGGGGATGACGTCA |
| | AATCATCATGCCCCTTATGACCTGGGCTACACACGTGCTACAATGGACGGTACAAAG |
| | AGCTGCAAGACCGCGAGGTG |
| Strain | CGAGCGAACAGAAAAGGAAGNTGCTCCTTTGACGTTAGCGGCGGACGGGTGAGTA |
| jl.77 | ACACGTGGGCAACCTACCCCTANTAGTTTGGGATAACTCCGGGAANCCGGGGCTAA |
| SEQ ID | TACCGAATAATCTCTTTTGCTTCATGGTNAAAGACTGAAAGACGGTTTCGGCTGTCG |
| NO: 2 | CTATAGGATGGGCCCGCGGCGCATTAGCTAGTTGGTGAGGTAACGGCTCACCAAG |
| | GCGACGATGCGTAGCCGACCTGAGAGGGTGATCGGCCACACTGGGACTGAGACAC |
| | CGGCCCAGACTCCTACGGGAGGCAGCAGTAGGGAATCTTCCACAATGGGCGAAAG |
| | CCTGATGGAGCAACGCCGCGTGAGTGAAGAAGGTTTTCGGATCGTAAAACTCTGTT |
| | GTAAGGGAAGAACAAGTACAGTAGTAACTGGCTGTACCTTGACGGTACCTTATTAGA |
| | AAGCCACGGCTAACTACGTGCCAGCAGCCGCGGTAATACGTAGGTGGCAAGCGTT |
| | GTCCGGAATTATTGGGCGTAAAGCGCGCGCAGGCGGTCCTTTAAGTCTGATGTGAA |
| | AGCCCACGGCTCAACCGTGGAGGGTCATTGGAAACTGGGGGACTTGAGTGCAGAA |
| | GAGGAAAGTGGAATTCCAAGTGTAGCGGTGAAATGCGTAGAGATTTGGAGGAACAC |
| | CAGTGGCGAAGGCGACTTTCTGGTCTGTAACTGACGCTGAGGCGCGAAAGCGTGG |
| | GGAGCAAACAGGATTAGATACCCTGGTAGTCCACGCCGTAAACGATGAGTGCTAAG |
| | TGTTAGGGGGTTTCCGCCCCTTAGTGCTGCAGCTAACGCATTAAGCACTCCGCCTG |
| | GGGAGTACGGTCGCAAGACTGAAACTCAAAGGAATTGACGGGGGCCCGCACAAGC |
| | GGTGGAGCATGTGGTTTAATTCGAAGCAACGCGAAGAACCTTACCAGGTCTTGACAT |
| | CCCGTTGACCACTGTAGAGATATAGTTTCCCCTTCGGGGGCAACGGTGACAGGTGG |
| | NGGCATGGTTGTCGTCAGCTCGTGTCGTGAGATGNTGGGGTTAAGTCCCGCAACGA |
| | GCGCAACCCTTGATCTTAGTTGCCATCATTTAGTTGGGCACTCTAAGGTGACTGCCG |
| | GTGACAAACCGGAGGAGG |
| Strain | AATACATGCAAGTCGAGCGAATGGATTAAGAGCTTGCTCTTATGAAGTTAGCGGCGG |
| jl.21 | ACGGGTGAGTAACACGTGGGTAACCTGCCCATAAGACTGGGATAACTCCGGGAAAC |
| SEQ ID | CGGGGCTAATACCGGATAACATTTTGAACCGCATGGTTCGAAATTGAAAGGCGGCTT |
| NO: 3 | CGGCTGTCACTTATGGATGGACCCGCGTCGCATTAGCTAGTTGGTGAGGTAACGGC |
| | TCACCAAGGCAACGATGCGTAGCCGACCTGAGAGGGTGATCGGCCACACTGGGAC |
| | TGAGACACGGCCCAGACTCCTACGGGAGGCAGCAGTAGGGAATCTTCCGCAATGG |
| | ACGAAAGTCTGACGGAGCAACGCCGCGTGAGTGATGAAGGCTTTCGGGTCGTAAAA |
| | CTCTGTTGTTAGGGAAGAACAAGTGCTAGTTGAATAAGCTGGCACCTTGACGGTACC |
| | TAACCAGAAAGCCACGGCTAACTACGTGCCAGCAGCCGCGGTAATACGTAGGTGGC |
| | AAGCGTTATCCGGAATTATTGGGCGTAAAGCGCGCGCAGGTGGTTTCTTAAGTCTG |
| | ATGTGAAAGCCCACGGCTCAACCGTGGAGGGTCATTGGAAACTGGGAGACTTGAGT |
| | GCAGAAGAGGAAAGTGGAATTCCATGTGTAGCGGTGAAATGCGTAGAGATATGGAG |
| | GAACACCAGTGGCGAAGGCGACTTTCTGGTCTGTAACTGACACTGAGGCGCGAAAG |
| | CGTGGGGAGCAAACAGGATTAGATACCCTGGTAGTCCACGCCGTAAACGATGAGTG |
| | CTAAGTGTTAGAGGGTTTCCGCCCTTTAGTGCTGAAGTTAACGCATTAAGCACTCCG |
| | CCTGGGGAGTACGGCCGCAAGGCTGAAACTCAAAGGAATTGACGGGGGCCCGCAC |
| | AAGCGGTGGAGCATGTGGTTTAATTCGAAGCAACGCGAAGAACCTTACCAGGTCTT |
| | GACATCCTCTGACAACCCTAGAGATAGGGCTTCTCCTTCGGGAGCAGAGTGACAGG |
| | TGGTGCATGGTTGTCGTCAGCTCGTGTCGTGAGATGTTGGGTTAAGTCCCGCAACG |
| | AGCGCAACCCTTGATCTTAGTTGCCATCATTAAGTTGGGCACTCTAAGGTGACTGCC |
| | GGTGACAAACCGGAGGAAGGTGGGGATGACGTCAAATCATCATGCCCCTTATGACC |
| | TGGGCTACACACGTGCTACAATGGACGGTACAAAGAGCTGCAAGACCGCGAG |
| Strain | GGATGGTTGTTTGAACCGCATGGTTCAGACATAAAAGGTGGCTTCGGCTACCACTTA |
| jl.83 | CAGATGGACCCGCGGCGCATTAGCTAGTTGGTGAGGTAACGGCTCACCAAGGCAA |
| SEQ ID | CGATGCGTAGCCGACCTGAGAGGGTGATCGGCCACACTGGGACTGAGACACGGCC |
| NO: 4 | CAGACTCCTACGGGAGGCAGCAGTAGGGAATCTTCCGCAATGGACGAAAGTCTGAC |
| | GGAGCAACGCCGCGTGAGTGATGAAGGTTTTCGGATCGTAAAGCTCTGTTGTTAGG |
| | GAAGAACAAGTGCCGTTCAAATAGGGCGGCACCTTGACGGTACCTAACCAGAAAGC |
| | CACGGCTAACTACGTGCCAGCAGCCGCGGTAATACGTAGGTGGCAAGCGTTGTCC |
| | GGAATTATTGGGCGTAAAGGGCTCGCAGGCGGTTTCTTAAGTCTGATGTGAAAGCC |
| | CCCGGCTCAACCGGGGAGGGTCATTGGAAACTGGGGAACTTGAGTGCAGAAGAGG |
| | AGAGTGGAATTCCACGTGTAGCGGTGAAATGCGTAGAGATGTGGAGGAACACCAGT |
| | GGCGAAGGCGACTCTCTGGTCTGTAACTGACGCTGAGGAGCGAAAGCGTGGGGAG |
| | CGAACAGGATTAGATACCCTGGTAGTCCACGCCGTAAACGATGAGTGCTAAGTGTTA |
| | GGGGGTTTCCGCCCCTTAGTGCTGCAGCTAACGCATTAAGCACTCCGCCTGGGGAG |
| | TACGGTCGCAAGACTGAAACTCAAAGGAATTGACGGGGGCCCGCACAAGCGGTGG |
| | AGCATGTGGTTTAATTCGAAGCAACGCGAAGAACCTTACCAGGTCTTGACATCCTCT |
| | GACAATCCTAGAGATAGGACGTCCCCTTCGGGGGCAGAGTGACAGGTGGTGCATG |
| | GTTGTCGTCAGCTCGTGTCGTGAGATGTTGGGGTTAAGTCCCGCAACGAGCGCAAC |
| | CCTTGATCTTAGTTGCCAGCATTCAGTTGGGCACTCTAAGGTGACTGCCGGTGACAA |
| | ACCGGAGGAAGGGTGGGGGATGACGTCAAATCATCATGCCCCTTATGACCTGGGCT |
| | ACACACGTGCTACAATGGACAGAACAAAGGGCAGCGAAACCGCGAGGTTAAGCCAA |
| | TCCCACAAATCTGTTCTCAGTTCGGATCGCAGTCTGCAACTCGACTGCGTGAAGCTG |
| | GAATCGCTAGTAATCGCGGATCA |
| Strain | CGTGGGTAACCTGCCTGTAAGACTGGGATAACTCCGGGAAACCGGGGCTAATACCG |
| jl.68 | GATGGTTGTTTGAACCGCATGGTTCAGACATAAAAGGTGGCTTCGGCTACCACTTAC |
| SEQ ID | AGATGGACCCGCGGCGCATTAGCTAGTTGGTGAGGTAACGGCTCACCAAGGCAAC |
| NO: 5 | GATGCGTAGCCGACCTGAGAGGGTGATCGGCCACACTGGGACTGAGACACGGCCC |
| | AGACTCCTACGGGAGGCAGCAGTAGGGAATCTTCCGCAATGGACGAAAGTCTGACG |
| | GAGCAACGCCGCGTGAGTGATGAAGGTTTTCGGATCGTAAAGCTCTGTTGTTAGGG |
| | AAGAACAAGTGCCGTTCAAATAGGGGGGCACCTTGACGGTACCTAACCAGAAAGCC |
| | ACGGCTAACTACGTGCCAGCAGCCGCGGTAATACGTAGGTGGCAAGCGTTGTCCG |
| | GAATTATTGGGCGTAAAGGGCTCGCAGGCGGTTTCTTAAGTCTGATGTGAAAGCCC |
| | CCGGCTCAACCGGGGAGGGTCATTGGAAACTGGGGAACTTGAGTGCAGAAGAGGA |
| | GAGTGGAATTCCACGTGTAGCGGTGAAATGCGTAGAGATGTGGAGGAACACCAGTG |
| | GCGAAGGCGACTCTCTGGTCTGTAACTGACGCTGAGGAGCGAAAGCGTGGGGAGC |
| | GAACAGGATTAGATACCCTGGTAGTCCACGCCGTAAACGATGAGTGCTAAGTGTTA |
| | GGGGGTTTCCGCCCCTTAGTGCTGCAGCTAACGCATTAAGCACTCCGCCTGGGGAG |
| | TACGGTCGCAAGACTGAAACTCAAAGGAATTGACGGGGGCCCGCACAAGCGGTGG |
| | AGCATGTGGTTTAATTCGAAGCAACGCGAAGAACCTTACCAGGTCTTGACATCCTCT |
| | GACAATCCTAGAGATAGGACGTCCCCTTCGGGGGCAGAGTGACAGGTGGTGCATG |
| | GTTGTCGTCAGCTCGTGTCGTGAGATGTTGGGGTTAAGTCCCGCAACGAGCGCAAC |
| | CCTTGATCTTAGTTGCCAGCATTCAGTTGGGCACTCTAAGGTGACTGCCGGTGACAA |
| | ACCGGAGGAAGGGTGGGGGGATGACGTCAAATCATCATGCCCCTTATGACCTGGG |
| | CTACACACGTGCTACAATGGACAGAACAAAGGGCAGCGAAACCGCGAGGTTAAGCC |
| | AATCCCACAAATCTGTTCTCAGTTCGGATCGCAGTCTGCAACTCGACTGCGTGAAGC |
| | TGGAATCGCTAGTAATCGCGGATCA |
| Strain | TAACCTACCTATAAGACTGGGATAACTTCGGGAAACCGGAGNTAATACCGGATAACA |
| jl.121 | TGTTGAACCGCATGGTTCAACAGTGAAAGACGGTCTTGCTGTCACTTATAGATGGAT |
| SEQ ID | CCGCGCCGCATTAGCTAGTTGGTAAGGTAACGGCTTACCAAGGCAACGATGCGTAG |
| NO: 6 | CCGACCTGAGAGGGTGATCGGCCACACTGGAACTGAGACACGGTCCAGACTCCTA |
| | CGGGAGGCAGCAGTAGGGAATCTTCCGCAATGGGCGAAAGCCTGACGGAGCAACG |
| | CCGCGTGAGTGAAGAAGGTCTTCGGATCGTAAAACTCTGTTATTAGGGAAGAACAAA |
| | TGTGTAAGTAACTATGCACGTCTTGACGGTACCTAATCAGAAAGCCACGGCTAACTA |
| | CGTGCCAGCAGCCGCGGTAATACGTAGGTGGCAAGCGTTATCCGGAATTATTGGGC |
| | GTAAAGCGCGCGTAGGCGGTTTTTTAAGTGTGATGTGAAAGCCCACGGCTCAACCG |
| | TGGAGGGTCATTGGAAACTGGAAAACTTGAGTGCAGAAGAGGAAAGTGGAATTCCA |
| | TGTGTAGCGGTGAAATGCGCAGAGATATGGAGGAACACCAGTGGCGAAGGCGACTT |
| | TCTGGTCTGTAACTGACGCTGATGTGCGAAAGCGTGGGGATCAAACAGGATTAGAT |
| | ACCCTGGTAGTCCACGCCGTAAACGATGAGTGCTAAGTGTTAGGGGGTTTCCGCCC |
| | CTTAGTGCTGCAGCTAACGCATTAAGCACTCCGCCTGGGGAGTACGACCGCAAGGT |
| | TGAAACTCAAAGGAATTGACGGGGACCCGCACAAGCGGTGGAGCATGTGGTTTAAT |
| | TCGAAGCAACGCGAAGAACCTTACCAAATCTTGACATCCTCTGACCCCTCTAGAGAT |
| | AGAGTTTTCCCCTTCGGGGGACAGAGTGACAGGTGGTGCATGGTTGTCGTCAGCTC |
| | GTGTCGTGAGATGTTGGGTTAAGTCCCGCAACGAGCGCAACCCTTAAGCTTAGTTG |
| | CCATCATTAAGTTGGGCACTCTAAGTTGACTGCCGGTGACAAACCGGAGGAAGGTG |
| | GGGATGACGTCNAATCATCNTGCCNCTTATGATTNGGGCTACACACGTGCTACAATG |
| | GACAATA |
| Strain | AATACATGCAAGTCGAGCGAACAGATAAGGAGCTTGCTCCTTTGACGTTAGCGGCG |
| jl.19 | GACGGGTGAGTAACACGTGGGTAACCTACNTATAAGACTGGAATAACTCCGGGAAA |
| SEQ ID | CCGGGGCTAATGCCGGATAACATTTAGAACCGCATGGTTCTAAAGTGAAAGATGGTT |
| NO: 16 | TTGCTATCACTTATAGATGGACCCGCGCCGTATTAGCTAGTTGGTAAGGTAACGGCT |
| | TACCAAGGCAACGATACGTAGCCGACCTGAGAGGGTGATCGGCCACACTGGAACTG |
| | AGACACGGTCCAGACTCCTACGGGAGGCAGCAGTAGGGAATCTTCCGCAATGGGC |
| | GAAAGCCTGACGGAGCAACGCCGCGTGAGTGATGAAGGTCTTCGGATCGTAAAACT |
| | CTGTTATTAGGGAAGAACAAATGTGTAAGTAACTATGCACGTCTTGACGGTACCTAA |
| | TCAGAAAGCCACGGCTAACTACGTGCCAGCAGCCGCGGTAATACGTAGGTGGCAAG |
| | CGTTATCCGGAATTATTGGGCGTAAAGCGCGCGTAGGCGGTTTCTTAAGTCTGATGT |
| | GAAAGCCCACGGCTCNNCCGTGGAGGGTCATTGGAAACTGGGAAACTTGAGTGCA |
| | GAAGAGGAAAGTGGAATTCCATGTGTAGCGGTGAAATGCGCAGAGATATGGAGGAA |
| | CACCAGTGGCGAAGGCGACTTTCTGGTCTGTAACTGACGCTGATGTGCGAAAGCGT |
| | GGGGATCAAACAGGATTAGATACCCTGGTAGTCCACGCCGTAAACGATGAGTGCTA |
| | AGTGTTAGGGGGTTTCCGCCCCTTAGTGCTGCAGCTAACGCATTAAGCACTCCGCC |
| | TGGGGAGTACGACCGCAAGGTTGAAACTCAAAGGAATTGACGGGGACCCGCACAA |
| | GCGGTGGAGCATGTGGTTTAATTCGAAGCAACGCGAAGAACCTTACCAAATCTTGAC |
| | ATCCTTTGACAACTCTAGAGATAGAGCCTTCCCCTTCGGGGGACAAAGTGACAGGT |
| | GGTGCATGGTTGTCGTCAGCTCGTGTCGTGAGATGTTGGGTTAAGTCCCGCAACGA |
| | GCGCAACCCTTAAACTTAGTTGCCAGCATTTAGTTGGGCACTCTAAGTTGACTGCCG |
| | GTGACAAACCGGAGGAAGGTGGGGATGACGTCAAATCATCATGCCCCTTATGATTT |
| | GGGCTACACACGTGCTACAATGGACAATACAAA |
| Strain | CGCATTAGCTAGTTGGTAAGGTAACGGCTTACCAAGGCAACGATGCGTAGCCGACC |
| jl.39 | TGAGAGGGTGATCGGCCACACTGGAACTGAGACACGGTCCAGACTCCTACGGGAG |
| SEQ ID | GCAGCAGTAGGGAATCTTCCGCAATGGGCGAAAGCCTGACGGAGCAACGCCGCGT |
| NO: 17 | GAGTGAAGAAGGTCTTCGGATCGTAAAACTCTGTTATTAGGGAAGAACAAATGTGTA |
| | AGTAACTATGCACGTCTTGACGGTACCTAATCAGAAAGCCACGGCTAACTACGTGCC |
| | AGCAGCCGCGGTAATACGTAGGTGGCAAGCGTTATCCGGAATTATTGGGCGTAAAG |
| | CGCGCGTAGGCGGTTTTTTAAGTCTGATGTGAAAGCCCACGGCTCAACCGTGGAGG |
| | GTCATTGGAAACTGGAAAACTTGAGTGCAGAAGAGGAAAGTGGAATTCCATGTGTAG |
| | CGGTGAAATGCGCAGAGATATGGAGGAACACCAGTGGCGAAGGCGACTTTCTGGTC |
| | TGTAACTGACGCTGATGTGCGAAAGCGTGGGGATCAAACAGGATTAGATACCCTGG |
| | TAGTCCACGCCGTAAACGATGAGTGCTAAGTGTTAGGGGGTTTCCGCCCCTTAGTG |
| | CTGCAGCTAACGCATTAAGCACTCCGCCTGGGGAGTACGACCGCAAGGTTGAAACT |
| | CAAAGGAATTGACGGGGACCCGCACAAGCGGTGGAGCATGTGGTTTAATTCGAAGC |
| | AACGCGAAGAACCTTACCAAATCTTGACATCCTCTGATCCCTCTAGAGATAGAGTTTT |
| | CCCCTTCGGGGGACAGAGTGACAGGTGGTGCATGGTTGTCGTCAGCTCGTGTCGT |
| | GAGATGTTGGGTTAAGTCCCGCAACGAGCGCAACCCTTAAGCTTAGTTGCCATCATT |
| | AAGTTGGGCACTCTAAGTTGACTGCCGGTGACAAACCGGAGGAAGGTGGGGATGA |
| | CGTCAAATCATCATGCCCCTTATGATTTGGGCTACACACGTGCTACAATGGACAATA |
| | CAAAGGGTAGCAAAACCGCGAGGTCAAGCAAATCCCATAAAGTTGTTCTCAGTTCG |
| | GATTGTAGTCTGCAACTCGACTACATGAAGCTGGAATCGCTAGTAATCG |
| Strain | CTGAGAGGGTGATCGGCCACACTGGGACTGAGACACGGCCCAGACTCCTACGGGA |
| jl.26 | GGCAGCAGTAGGGAATCTTCCGCAATGGACGAAAGTCTGACGGAGCAACGCCGCG |
| SEQ ID | TGAGTGATGAAGGCTTTCGGGTCGTAAAACTCTGTTGTTAGGGAAGAACAAGTGCTA |
| NO: 18 | GTTGAATAAGCTGGCACCTTGACGGTACCTAACCAGAAAGCCACGGCTAACTACGT |
| | GCCAGCAGCCGCGGTAATACGTAGGTGGCAAGCGTTATCCGGAATTATTGGGCGTA |
| | AAGCGCGCGCAGGTGGTTTCTTAAGTCTGATGTGAAAGCCCACGGCTCAACCGTGG |
| | AGGGTCATTGGAAACTGGGAGACTTGAGTGCAGAAGAGGAAAGTGGAATTCCATGT |
| | GTAGCGGTGAAATGCGTAGAGATATGGAGGAACACCAGTGGCGAAGGCGACTTTCT |
| | GGTCTGTAACTGACACTGAGGCGCGAAAGCGTGGGGAGCAAACAGGATTAGATACC |
| | CTGGTAGTCCACGCCGTAAACGATGAGTGCTAAGTGTTAGAGGGTTTCCGCCCTTTA |
| | GTGCTGAAGTTAACGCATTAAGCACTCCGCCTGGGGAGTACGGCCGCAAGGCTGAA |
| | ACTCAAAGGAATTGACGGGGGCCCGCACAAGCGGTGGAGCATGTGGTTTAATTC |
| Strain | CATGGTTCGAAATTGAAAGGCGGCTTCGGCTGTCACTTATGGATGGACCCGCGTCG |
| jl.116 | CATTAGCTAGTTGGTGAGGTAACGGCTCACCAAGGCAACGATGCGTAGCCGACCTG |
| SEQ ID | AGAGGGTGATCGGCCACACTGGGACTGAGACACGGCCCAGACTCCTACGGGAGGC |
| NO: 19 | AGCAGTAGGGAATCTTCCGCAATGGACGAAAGTCTGACGGAGCAACGCCGCGTGA |
| | GTGATGAAGGCTTTCGGGTCGTAAAACTCTGTTGTTAGGGAAGAACAAGTGCTAGTT |
| | GAATAAGCTGGCACCTTGACGGTACCTAACCAGAAAGCCACGGCTAACTACGTGCC |
| | AGCAGCCGCGGTAATACGTAGGTGGCAAGCGTTATCCGGAATTATTGGGCGTAAAG |
| | CGCGCGCAGGTGGTTTCTTAAGTCTGATGTGAAAGCCCACGGCTCAACCGTGGAGG |
| | GTCATTGGAAACTGGGAGACTTGAGTGCAGAAGAGGAAAGTGGAATTCCATGTGTA |
| | GCGGTGAAATGCGTAGAGATATGGAGGAACACCAGTGGCGAAGGCGACTTTCTGGT |
| | CTGTAACTGACACTGAGGCGCGAAAGCGTGGGGAGCAAACAGGATTAGATACCCTG |
| | GTAGTCCACGCCGTAAACGATGAGTGCTAAGTGTTAGAGGGTTTCCGCCCTTTAGT |
| | GCTGAAGTTAACGCATTAAGCACTCCGCCTGGGGAGTACGGCCGCAAGGCTGAAAC |
| | TCAAAGGAATTGACGGGGGCCCGCACAAGCGGTGGAGCATGTGGTTTAATTCGAAG |
| | CAACGCGAAGAACCTTACCAGGTCTTGACATCCTCTGAAAACCCTAGAGATAGGGCT |
| | TCTCCTTCGGGAGCAGAGTGACAGGTGGTGCATGGTTGTCGTCAGCTCGTGTCGTG |
| | AGATGTTGGGTTAAGTCCCGCAACGAGCGCAACCCTTGATCTTAGTTGCCATCATTA |
| | AGTTGGGCACTCTAAGGTGACTGCCGGTGACAAACCGGAGGAAGGTGGGGATGAC |
| | GTCAAATCATCATGCCCCTTATGACCTGGGCTACACACGTGCTACAATGGACGGTAC |
| | AAA |
| Strain | AACCGGGGCTAATACCGAATAACACTTTTGACCTCATGGTCGAATGTTAAAAGACGG |
| jl.119 | TTTCGGCTGTCACTACAGGATGGGCCCGCGGCGCATTAGCTAGTTGGTGAGGTAAC |
| SEQ ID | GGCTCACCAAGGCAACGATGCGTAGCCGACCTGAGAGGGTGATCGGCCACACTGG |
| NO: 20 | GACTGAGACACGGCCCAGACTCCTACGGGAGGCAGCAGTAGGGAATCTTCCACAAT |
| | GGACGAAAGTCTGATGGAGCAACGCCGCGTGAGTGAAGAAGGATTTCGGTTCGTAA |
| | AACTCTGTTGCAAGGGAAGAACAAGTAGCGTAGTAACTGGCGCTACCTTGACGGTA |
| | CCTTGTTAGAAAGCCACGGCTAACTACGTGCCAGCAGCCGCGGTAATACGTAGGTG |
| | GCAAGCGTTGTCCGGAATTATTGGGCGTAAAGCGCGCGCAGGTGGTTTCTTAAGTC |
| | TGATGTGAAAGCCCACGGCTCAACCGTGGAGGGTCATTGGAAACTGGGAAACTTGA |
| | GTGCAGAAGAGGATAGTGGAATTCCAAGTGTAGCGGTGAAATGCGTAGAGATTTGG |
| | AGGAACACCAGTGGCGAAGGCGACTATCTGGTCTGTAACTGACACTGAGGCGCGAA |
| | AGCGTGGGGAGCAAACAGGATTAGATACCCTGGTAGTCCACGCCGTAAACGATGAG |
| | TGCTAAGTGTTGGGGGGTTTCCGCCCCTCAGTGCTGCAGCTAACGCATTAAGCACT |
| | CCGCCTGGGGAGTACGGTCGCAAGACTGAAACTCAAAGGAATTGACGGGGGCCCG |
| | CACAAGCGGTGGAGCATGTGGTTTAATTCGAAGCAACGCGAAGAACCTTACCAGGT |
| | CTTGACATCCCATTGACCACTGTAGAGATACAGTTTTCCCTTCGGGGACAACGGTGA |
| | CAGGTGGTGCATGGTTGTCGTCAGCTCGTGTCGTGAGATGTTGGGTTAAGTCCCGC |
| | AACGAGCGCAACCCTTATTCTTAGTTGCCATCATTTAGTTGGGCACTCTAAGGAGAC |
| | TGCCGGTGACAAACCGGAGGAAGGTGGGGATGACGTCAAATCATCATGCCCCTTAT |
| | GACCTGGGCTACACACGTGCTACAATGGACGGTACAAACGGTTGCCAACCCGCGAG |
| | GGGGAGCTAATCCGATAAAACCG |
| Strain | TGTGGATGGGCTCACGGCCTATCAGCTTGTTGGTGGGGTAATGGCCTACCAAGGCG |
| jl.45 | ACGACGGGTAGCCGGCCTGAGAGGGTGACCGGCCACACTGGGACTGAGACACGG |
| SEQ ID | CCCAGACTCCTACGGGAGGCAGCAGTGGGGAATATTGCACAATGGGCGCAAGCCT |
| NO: 21 | GATGCAGCGACGCCGCGTGAGGGATGACGGCCTTCGGGTTGTAAACCTCTTTCAGC |
| | ACGGAAGAAGCGAAAGTGACGGTACGTGCAGAAGAAGCGCCGGCTAACTACGTGC |
| | CAGCAGCCGCGGTAATACGTAGGGCGCAAGCGTTGTCCGGAATTATTGGGCGTAAA |
| | GAGCTCGTAGGCGGTTTGTCGCGTCTGCTGTGAAAGCCCGGGGCTTAACCCCGGG |
| | TGTGCAGTGGGTACGGGCAGACTTGAGTGCAGTAGGGGAGACTGGAACTCCTGGT |
| | GTAGCGGTGAAATGCGCAGATATCAGGAAGAACACCGATGGCGAAGGCAGGTCTCT |
| | GGGCTGTTACTGACGCTGAGGAGCGAAAGCATGGGGAGCGAACAGGATTAGATAC |
| | CCTGGTAGTCCATGCCGTAAACGTTGGGCACTAGGTGTGGGGGACATTCCACGTTT |
| | TCCGCGCCGTAGCTAACGCATTAAGTGCCCCGCCTGGGGAGTACGGCCGCAAGGC |
| | TAAAACTCAAAGGAATTGACGGGGGCCCGCACAAGCGGCGGAGCATGCGGATTAAT |
| | TCGATGCAACGCGAAGAACCTTACCAAGGCTTGACATACACCGGATCGGCTCAGAG |
| | ATGAGTTTTCCTCCTTGTGGGGCTGGTGTACAGGTGGTGCATGGTTGTCGTCAGCT |
| | CGTGTCGTGAGATGTTGGGTTAAGTCCCGCAACGAGCGCAACCCTCGTTCTATGTT |
| | GCCAGCACGTGATGGTGGGGACTCATAGGAGACTGCCGGGGTCAACTCGGAGGAA |
| | GGTGGGGATGACGTCAAATCATCATGCCCCTTAT |

TABLE 7
Identifier Sequences of Isolated Strains

| | | |
|---|---|---|
| Query | Percent | |
| Coverage | Identity | |
| to closest | to closest | |
| database | database | |
| match | match | Sequence |

Strain jl.77

| | | | |
|---|---|---|---|
| Identifier | 99 | 77.759 | TTCTATCACCAAAAACAGGTCAATTAACAGTAGGGG |
| Sequence 1 | | | ATACACAAGACTTCCAGTTGGTAGAAAACCTGTCGG |
| (SEQ ID NO: 7) | | | ATGGGAGCAATCAAGACCAAACAGGAAATACAACAT |
| | | | TTACTGTTAGCGATTCATCCATTGCCACAATACAAAA |
| | | | CAACAAACTAACAGCGGTTGCTCCAGGGACCGTGA |
| | | | CGGTAACGGCAACCTATGGTAGCTCATCCGACACA |
| | | | GCAACAATTACGGTGAAAAAGCCTACACCAGTTCCG |
| | | | CCATCGGTACCACAGCCTGTAAATCCAGTGGAACC |
| | | | AAAGCCTTCAGGTGCAATAGACATTATTCGTACTGT |
| | | | TGAACAAGGTATCGTGAAGTATCGAGCAGACGTATC |
| | | | ATTGAATCATGTCCAAATGCTAGTACAACAAATGAC |
| | | | TAATCAAGATGCACGAACAATTCGTCTCGTGTACCC |
| | | | AGCAGAAACAGCAACTGCAAAGGCTTATTTGAATCT |
| | | | TTTTAGAAACGCAGGATTGTTTTTATACAATCAAAAT |
| | | | ACGGATCTTTTCATACAAACAGAGTTAGCACAAATG |
| | | | AGGATTCCTTTTAAATCATTCGATGGCGTGACAGAG |
| | | | GATGTGTACTTCCATCTAGCTCCTGTCAAGGGACCA |
| | | | CAACAGGATATGATTCATACGAATGCCCTAAACAAT |
| | | | GAACAAATCCAGAAAGCAATGCCAGATAGAGCGAT |
| | | | AATTTCATTACTAGGTACACCCGTAAGAATCGAAAC |
| | | | GAATCTACAAAATCGCCCAGTAATGATTACGTTACC |
| | | | GATTTCGTCTGAGTTAACAGAAGATCAAATTGCCTC |
| | | | ACTACTTATCTATGTTGAGCATAGTGACGAAACAAC |
| | | | GGAAGTAAAACATGGGCGTATAGTGGAGTTTGTAC |
| | | | CTGGGGTGAAAGGTTTCCAGTTTGAAGTTGATCACT |
| | | | TCTCAACATATAGCTTAGTATATACTTCAGAAGTTCA |
| | | | AGAAGCTGAGGAAGAAGTAGAGCAAATAGCACCAT |
| | | | ATATTCAAGGTTTCCCAGATGGCACATTTAAACCAA |
| | | | ATGCTTCGGTTACACGTGCACAAATGGCAACAATGT |
| | | | TAGCACGTTTTTTAACGAATGGTGACATACCAACGG |
| | | | CAAGTGCAACTTTTAAAGATACAAAGAATCATCCTTC |
| | | | TAAAGATGCGATTGAATTAGTGAAAGAAATTGGTTT |
| | | | ATTTAACGGTATAACGGATACAACATTTAATCCAAAT |
| | | | GGGACGATTACAA |
| Identifier | 99 | 77.188 | CGCTACTGGCGTTACCGGGGCTACTGGTGTTACTG |
| Sequence 2 | | | GCGCTACCGGCGCTACCGGCGCTACCGGCGTTACT |
| (SEQ ID NO: 8) | | | GGCGTTACTGGTTCTACGGGTGTAACCGGCGCTAC |
| | | | TGGTTCTACCGGCGCTACCGGCGTTACTGGAGTAA |
| | | | CTGGGGCTACTGGTGTTGCTGGTTCTACGGGCGCT |
| | | | ACTGGCGCAATCGGACCTACTGGCGCTACTGGCGT |
| | | | TACTGGCGCTACCGGGGCTACTGGGGTAACTGGG |
| | | | GCTACCGGCGTTACTGGGGTAACCGGCGCTACTGG |
| | | | CGCTACTGGTGTAACCGGGGCTACCGGGGCTACTG |
| | | | GCTTGACTGGCGCAATCGGACCTACTGGCACCACT |
| | | | GGGATAACTGGGGCTACCGGCGTTACTGGCGTTAC |
| | | | TGGCGCTACCGGCGTTGCTGGGGCTACGGGTGAG |
| | | | ACTGGCGCTACTGGGGTAACTGGGGCTACCGGTGT |
| | | | TGCTGGGGCTACGGGCGCTACGGGCGTTACTGGC |
| | | | GCAATCGGACCTACTGGCGCTACTGGTGTAACCGG |
| | | | GGCTACCGGCGTTACTGGCGTTACTGGGGCTACCG |
| | | | GCGTTACTGGGGTAACCGGCGCTACTGGCTTGACT |
| | | | GGTGCAATCGGACCTACTGGCGCTACTGGAGTAAC |
| | | | CGGGGCTACCGGCGTTACTGGGGCTACTGGCGTTA |
| | | | CTGGGGTAACTGGGGCTACCGGCGTTACTGGCGTT |
| | | | ACTGGCGCTACCGGCGTTGCTGGCGTTACCGGCGT |
| | | | TACTGGCGCTACTGGCGTTACTGGTTCTACGGGAG |
| | | | TGACCGGCGCTACTGGTATTACTGGTTCTACGGGT |
| | | | GTAACCGGGGCTACTGGCGCTACTGGTTCTACTGG |
| | | | GGTAACCGGCGTTACTGGTTCTACGGGTGAGACTG |
| | | | GGGTAACTGGGGCTACCGGCATTACTGGAGTAACC |
| | | | GGCGCTACTGGCATTACTGGAGTAACCGGCGCTAC |
| | | | TGGCACTACTGGAGTAACTGGCGCTACCGGCGTTA |
| | | | CTGGCGCTACCGGCGTTGCTGGGGCTACGGGTGA |
| | | | GACCGGCGCTACTGGCACTACTGGAGTAACTGGGG |
| | | | CTACCGGTGTTGCTGGTTCTACGGGCGCGACTGGC |
| | | | GCAATCGGACCTACTGGCGTTACTGGTGTAACTGG |
| | | | GGCTACCGGCGCAATCGGACCTACTGGCGTTACTG |
| | | | GAGCTACCGGCGTTGCTGGGGTAACTGGGGCTACT |
| | | | GGCGTTACTGGTGC |

Strain jl.21

| | | | |
|---|---|---|---|
| Identifier | 99 | 81.925 | TATAAATCCGTAATGCTTTTTTCTTTTGTTCGTTTAC |
| Sequence 1 | | | ATTCATAAAATCGTGTAACCTTTTAAGCTTTTTCCCT |
| (SEQ ID NO: 9) | | | TTCTGCTCTTCTACATTGCTTTGAAGTTTTAATGTAA |
| | | | CATACTGCCCTTTTTTCACGAGTAAATCAATAATAAA |
| | | | TCTACGAAAATCATGATATTGAATAGGTACATGATTT |
| | | | ACACAACCATACTTTCCTGCTCTTCTATACTGTGAG |
| | | | CACTTTAAATATTTCCATTCAGTCCGTTCACCATTCT |
| | | | TCTTTCGAAGATAGGATTGTACGATAACCATATTTGA |
| | | | ACCACAAATCGCGCATTTTGCTAAATTACGAAATTC |
| | | | ATTCCAAGCTGTGATTTTTGTTTTACTCATAATAATG |
| | | | TCTTTTTGATTTGCTCGATTCCATATCTCTTCATCTA |
| | | | CAATTTTGGGATGGTGATTAGGAAAAACAAACCACT |
| | | | TTTCTCTCGGATTGCGAATTTGTTTTTTCCTACCGTT |
| | | | CACCTTGACCGAGGTATATTGATTTAAAATAAACGT |
| | | | CCCTTTGTAAATTGGATTTTGAAGAATACGCTGAAC |
| | | | GGATGTTATTTGCCAATTTTTTTTAGTTTTAGATTTTA |
| | | | TCCCCATTTTATTTAACTCATTTGAAATTTTTTTATAT |
| | | | CCCCACCCATTGATATACCAACTATATATCCTTCGA |
| | | | ACAACATTAGATTCCTCTTCATTAATAATCAACTTTT |
| | | | GATTAATACGGTCATACCCATACGGTACTTTACCAA |
| | | | TATGCTCACCTCTTCTTACTTTCACCGCCAAAGCAG |
| | | | CAGTAATACTCGAAGATAATGTCCGACTATACTGCG |
| | | | CCGAAAATAAAGACCATAACTCAAATGCCATATCAT |
| | | | TTTTCCCAGCTTTTACAGAATCATAGCCTTCTTCCAC |
| | | | AGAAACAATTCTAACATTATGAGCTAAAAGCACTTCT |
| | | | CTTATCTCTAATGAGTCCTTTAAATCACGCGCTAAAC |
| | | | GAGAAATAGACTTAAAGACAACCATATCAATTTCTTT |
| | | | CGCCTTCGCCTTTTGTAATAATAATTGAATAGCAGG |
| | | | ACGATCCATAAATAAAGTACCACTCATCCCCTCATC |
| | | | TTTAAAAACACATTTTTCATTCCACATAAATCCATTA |
| | | | CGCTCCAACCAATTCCTACAAATATCTATTTGGTTTT |
| | | | CAATCGAGGAAACTTGTTCATCACGATCTGTTGATA |
| | | | CGCGAACATAAACAGCATAAGATTCC |
| Identifier | 99 | 74.631 | CTGTTTTAGTCCTCCTAAGTAATATTTCATAACTTCA |
| Sequence 2 | | | TATTTTGCATGTGTATCATCAGATAGTAGAATGTCAA |
| (SEQ ID NO: 10) | | | TTTCATATTCTAAAATCGGCAACTTATGATATATGAA |
| | | | GTCGAAAAAATCTGTTTCGGTTGTGAATTCAAATTGT |
| | | | TTTAAAGTATCTTTGAAAAATCTTTCTCGATTGAGTA |
| | | | ATAATTGCAACAAATGATTTTCTAAATGCTTTATTTCT |
| | | | CGCATTTTTTTATGTATGGCTTTTTGTTTAGCTATTT |
| | | | CTTTTTGCATGTTTTTATTATTTAGATTTACATCTAAA |
| | | | TTCAATTCTTTTAAAAGAAAAGATTCTGCTTGTTTGT |
| | | | GCGTCCATCCATGCAATATCATCAGACCTGAAAATT |
| | | | GGTCTATTGGTCTTCTATTCCCGCACCCTGCGAAAC |
| | | | AATAACAGTTGTTGTCTGATACGTTCACAACCATGC |
| | | | TATCTTTGTTATCTGCATGGAAGGGACACTTGCACA |
| | | | TGTAATTTTTTCCCCGCTTTCTAACAGTTACCCCGCA |
| | | | ATAGCTTTCTAAGAAATTTATAATAGGAAGTTTACTT |
| | | | TTAATTAGATCGCTTTTTTGCATATCATCACACTTCT |
| | | | TTATCTTTGAAAATAATTTTGATATGTTTAGCTTGGTT |
| | | | TTTTCCTCGTTGATAGAAAACATCTGTAAGACCTTTT |
| | | | TCCTCCAAACCTTTGAAGGCTCGTCCAACGCTAGAG |
| | | | CGTGATAAATTGCAATGTTCTATTACATCTTCAATCT |
| | | | TAATTTTTTCGTGTTGTTCTGCACAACCTTGCTGATG |
| | | | TAGATAGAGCAATACAAGTTTTTCAGTAGGTGTTGT |
| | | | TTCTTGGACTTCGAAAATGTCTTTTATCGTCAATATT |
| | | | CTGCTCCTCCTGTCAAATTTCTTTATGAATTTAACTT |
| | | | TAACAGATATTTTCTTATGCGTCAATATGACACATAA |
| | | | GAATTATATAACGAAAATAATTAAATCCTTTAATATAA |
| | | | ACGATTTCTAGCGAGTAATAAAAACGATTCCTTGATT |
| | | | CATTTTCGGAAACACCTGACGTCTTCCCTACGATTC |
| | | | CTTATATTTTTTGCGGGGATTATTCATGTTTCTTTGT |
| | | | CAATCAGCAAGGACAAACAATCCTCGCTATTTTGAG |
| | | | AGTGTATTTTCATATTCCTTCATGATTTTGTAGAATG |
| | | | ATGTTTTTTTGAGATTTAGCATTTGACTGAATTCTAC |
| | | | ACCTTTGATTTCTTTGCTTT |

Strain jl.83

| | | | |
|---|---|---|---|
| Identifier | 100 | 88.353 | GTTTCTCCCGTTGCTCCGGTTACTCCTGTTGCCCCA |
| Sequence 1 | | | GTTGGGCCGGTTGCTCCGATTGAACCTGTTGCTCC |
| (SEQ ID NO: 11) | | | GGTTGGACCGGTCTCTCCCGTTGGGCCGATTTCTC |
| | | | CAGTTACACCGGTTGCGCCGGTTTCTCCCGTTGAA |
| | | | CCAATTGAGCCGGTTACTCCTGTTGCCCCTGTTGCT |
| | | | CCAGTTACACCAGTCAAGCCGGTTGGGCCTGTTATT |
| | | | CCTGTTATTCCTGTTGCTCCGGTTGAACCGGATGCA |
| | | | CCCGTCGCCCCGGTTTCTCCTGTCGATCCCGTTGG |
| | | | ACCAGTCTCCCCAGTTGACCCGGTTGATCCTGTCG |
| | | | CGCCTGTTACTCCCGTTGGGCCTGTTGGGCCTGTT |
| | | | GAGCCGGTCGTTCCCGTTGCTCCTGTCGGCCCGGT |
| | | | TATTCCTGTCGCACCGGTTTCTCCGGTTACTCCCGT |
| | | | CGCCCCGGTTACTCCAGTTGAACCCGTTTCTCCTGT |
| | | | CGATCCTGTTGATCCGGTTACACCAGTTGAGCCCG |
| | | | TGACCCCAGTCGGGCCAGTCACTCCTGTTGATCCT |
| | | | GTTGCACCAGTTACTCCGGTTGGGCCTGTTGAACC |
| | | | TGTCGATCCCGTTGCGCCGGTTGGGCCTGCTGCGC |
| | | | CAGTTGAGCCGGTTGAGCCAGTTGGCCCGATTTCT |
| | | | CCAGTTGAACCGGTCGCTCCTGTCTCCCCGGTCGC |
| | | | TCCTGTTGGGCCGATCGGGCCAGTAGCCCCTGTGG |
| | | | CTCCCGTTATCCCCGTTGCCCCAGTCACTCCAGTTG |
| | | | GACCCGTTTCTCCCGTTGAGCCAGTTGCTCCTGTTT |
| | | | CTCCTGTTGGACCAGTCTCCCCAGTTGACCCGGTT |
| | | | GATCCGGTTTCTCCTGTCGATCCCGTTGCGCCGGT |
| | | | TATTCCTGTTACACCGGTCTCTCCTATTGCGCCGGT |
| | | | TGGGCCTGTTACTCCAGTCGCTCCGGTTGGACCGG |
| | | | TTTCTCCGGTTGAACCAGTTGACCCTGCTGCTCCTG |
| | | | TTGGTCCAGTTACTCCCGTTGAACCAGTTGATCCCG |
| | | | TTATACCAGTTACCCCAGTCGCTCCTGTCGATCCCA |
| | | | TTGCACCCGTTGAGCCGGTGGCTCCTGTTACTCCC |
| | | | GTTGCTCCGGTTGAGCCGGTTGGGCCGGTTGGGC |
| | | | CGGTTACACCCGTGACCCCAGTCGGGCCAGTAACT |
| | | | CCTGTTGCCCCTGTTACTCCGGTCGATCCCGTTTCT |
| | | | CCTGTTGCACCTGTTACTCCGGTCTCTCCTGTT |
| Identifier | 99 | 93.709 | AAAACTGTCAGTGTAATGGATACACCTGATTTCCCG |
| Sequence 2 | | | GAGATTTTTATTCCAACAGCTAAAGGCCAAGGTCGT |
| (SEQ ID NO: 12) | | | AACTTGCAAGAACTAAAGGAAACTTCTGATATTAACT |
| | | | GGACATTTATCAGTCCTTCAGCGGTATTTGACCCAG |
| | | | ACGGGAAAAGAACTGGATTTTATCAGTCAGGAAAAG |
| | | | ATCATCTTCTTGTGAATTCGAAAGGCGAAAGTTATAT |
| | | | CAGCTATGCAGACTATGCAATTGCAGTATTGGATGA |
| | | | AATTGAAAATCCAAAACATATAAATGAACGCTTTACA |
| | | | GTTGTTGGAGAAGCTGAATAAGTGATTGAGTAGAAA |
| | | | CAAAGGGTACCATTAGTGTCATCGCTAATGGTATCT |
| | | | TCCTTCTAAGAATGTACGGAGACATTGAAAACTTTG |
| | | | CAATCAGAGTAGCTGATTATTAAAAAGGGGATTTAT |
| | | | CACGTTCCAATGAATATTATCGAGCTGATGACAGAG |
| | | | TAGGTGCTTAGTGAGTTGGACCAAAAAAATATGTAT |
| | | | GTGTATAAAAAGCTCTGACTAAGACCTTTTGGATTA |
| | | | GTTAGAGCTTCTTTATTTGACTGTGTTACCCAGTGTA |
| | | | ATCCTCCGTATTGTTTAATACACTTTAGTTTGAAAAT |
| | | | CCGGGAAACTCTTCTACAGTTCCTATTTCGTGTTTA |
| | | | AAGTGTGAATATTCCAGCGGGGAACTTTACTGAAAT |
| | | | GAGGGGTATTTTCGAAAACACACCAGTATGCTCTAT |
| | | | GACCTGCCGTCAATGACATTAAAGAAATGATTTGAT |
| | | | GTAAAAAGGAGCGATATTCGTTAAAATTTAGGTAGT |
| | | | AGCCATACATAGCCATATAAAACACATGATTAATTCT |
| | | | AAATTCTTTACTGATCGTTCTTAAATTTGATATACTT |
| | | | GAAATACGTTAGATCTAACTGTTTTGCTACTCAAATG |
| | | | AGAATGATCGTTCTTTAATTATTTAAGAATGAGGGA |
| | | | GGGATGAATGCATCAATGATTCTCAGGCCTTTCTCA |
| | | | GAATAAAGAGGTAATACATTCTAAGGTGGGATTACA |
| | | | TGGCTAGAAATAAAGAATTTGATGAAAAAAAAGCAT |
| | | | TAAGAAAAGCAATGGATCTTTTCTGGGAACAGGGTT |
| | | | ATGAAAAAACATCCATGCAAGACTTGGTGGACCATA |
| | | | TGGGCATTCACCGCAGAAGTATTTATGATACATTTG |
| | | | GCGACAAACATACTTTATTTATGCGAGCCTTAAGTC |
| | | | AGTA |

Strain jl.68

| | | | |
|---|---|---|---|
| Identifier | 100 | 88.353 | GTTTCTCCCGTTGCTCCGGTTACTCCTGTTGCCCCA |
| Sequence 1 | | | GTTGGGCCGGTTGCTCCGATTGAACCTGTTGCTCC |
| (SEQ ID NO: 13) | | | GGTTGGACCGGTCTCTCCCGTTGGGCCGATTTCTC |
| | | | CAGTTACACCGGTTGCGCCGGTTTCTCCCGTTGAA |
| | | | CCAATTGAGCCGGTTACTCCTGTTGCCCCTGTTGCT |
| | | | CCAGTTACACCAGTCAAGCCGGTTGGGCCTGTTATT |
| | | | CCTGTTATTCCTGTTGCTCCGGTTGAACCGGATGCA |
| | | | CCCGTCGCCCCGGTTTCTCCTGTCGATCCCGTTGG |
| | | | ACCAGTCTCCCCAGTTGACCCGGTTGATCCTGTCG |
| | | | CGCCTGTTACTCCCGTTGGGCCTGTTGGGCCTGTT |
| | | | GAGCCGGTCGTTCCCGTTGCTCCTGTCGGCCCGGT |
| | | | TATTCCTGTCGCACCGGTTTCTCCGGTTACTCCCGT |
| | | | CGCCCCGGTTACTCCAGTTGAACCCGTTTCTCCTGT |
| | | | CGATCCTGTTGATCCGGTTACACCAGTTGAGCCCG |
| | | | TGACCCCAGTCGGGCCAGTCACTCCTGTTGATCCT |
| | | | GTTGCACCAGTTACTCCGGTTGGGCCTGTTGAACC |
| | | | TGTCGATCCCGTTGCGCCGGTTGGGCCTGCTGCGC |
| | | | CAGTTGAGCCGGTTGAGCCAGTTGGCCCGATTTCT |
| | | | CCAGTTGAACCGGTCGCTCCTGTCTCCCCGGTCGC |
| | | | TCCTGTTGGGCCGATCGGGCCAGTAGCCCCTGTGG |
| | | | CTCCCGTTATCCCCGTTGCCCCAGTCACTCCAGTTG |
| | | | GACCCGTTTCTCCCGTTGAGCCAGTTGCTCCTGTTT |
| | | | CTCCTGTTGGACCAGTCTCCCCAGTTGACCCGGTT |
| | | | GATCCGGTTTCTCCTGTCGATCCCGTTGCGCCGGT |
| | | | TATTCCTGTTACACCGGTCTCTCCTATTGCGCCGGT |
| | | | TGGGCCTGTTACTCCAGTCGCTCCGGTTGGACCGG |
| | | | TTTCTCCGGTTGAACCAGTTGACCCTGCTGCTCCTG |
| | | | TTGGTCCAGTTACTCCCGTTGAACCAGTTGATCCCG |
| | | | TTATACCAGTTACCCCAGTCGCTCCTGTCGATCCCA |
| | | | TTGCACCCGTTGAGCCGGTGGCTCCTGTTACTCCC |
| | | | GTTGCTCCGGTTGAGCCGGTTGGGCCGGTTGGGC |
| | | | CGGTTACACCCGTGACCCCAGTCGGGCCAGTAACT |
| | | | CCTGTTGCCCCTGTTACTCCGGTCGATCCCGTTTCT |
| | | | CCTGTTGCACCTGTTACTCCGGTCTCTCCTGTT |

Strain jl.121

| | | | |
|---|---|---|---|
| Identifier | 100 | 90.884 | AATATAAACAAATTGTGCTAAATCCGCTTTATAGTAT |
| Sequence 1 | | | AAGTAACAAAACGATAGATAAACAAACAACACAAGT |
| (SEQ ID NO: 14) | | | TCATTCATCGTTTTAAATTAATTAACTCACGCATTAT |
| | | | CACATTTTACTGAAGGAGTGTTTGTAATGGAAAAATT |
| | | | ATTCGACGCAATTAGAAACACAGTCGATGCTGGAAT |
| | | | CAACCAAGATTGGACAAAATTAGGAACTAGCATTGT |
| | | | TGACATCGTTGACAATGGTGTAAAAGTTATTTCTAAA |
| | | | TTTATTGGTGCATAATTCAGATTATTAATATCGTTTTA |
| | | | ATAATAAAGGAGAGATTATAATGCAAAAATTAGCAG |
| | | | AAGCAATCGCAAACACAGTAAAAGCAGGACAAGAC |
| | | | CATGATTGGACAAAATTAGGTACAAGCATCGTTGAT |
| | | | ATCGTAGAAAACGGTGTAAGTGCATTAACTAAAGTA |
| | | | TTCGGTGGTTAATTTTCGATAAATAAGAACTTAATTA |
| | | | TAAATAAAATAAACTAAAGGAGAGACTATAATGACTA |
| | | | AATTAGCAGAAGCAATCGCAAACGCAGTAAAAGCA |
| | | | GGACAAGACCAAGATTGGGCAAAATTAGGTACAAG |
| | | | CATCGTAGGTATCGCAGAAAACGGAATCGGTTTATT |
| | | | AGGTAAAGTATTCGGATTCTAATATATGATTAGATG |
| | | | GACCAGGGCAAGCGCTCTGGTCTTTTTTTATTTGCA |
| | | | GATTATTTGTTATTATTAAAAAGAATAAGAAGTTTGT |
| | | | TCATTAAAATTTGAAACGGGGATAAATTGTGACATAT |
| | | | AAACATATCTTATTAGATTTCGATGATACAATAGTTG |
| | | | ATTTTTACGATGCAGAGGAAAAGGCATTTTATAATAT |
| | | | GGCAAAACATTACGGTCATTTTCCGACTAAACAAGA |
| | | | TTTCCAACATTTTAGAAAGGTCAATCAAGCACACTG |
| | | | GGAAGCTTTTCAACAAAATGAATTGACGAAAGAACA |
| | | | AGTTTTATCTCATCGATTTATTAATTATTTCAATGATT |
| | | | ATTACATAGAAGTAGATGGTAAAGAAGCGGATGAGA |
| | | | TATTTAGAGATGAATTAGCTAAAGCGCCGCTTAAAT |
| | | | TTTTCGATCAAACGATTGAAACTATTAACCAGCTGAA |
| | | | AGATAAACATTCATTATATATTGTTACGAATGGTGTG |
| | | | ACAATCACGCAACAGCGTCGTATTGCTCAGACAAAT |
| | | | TTTAATGATATATTTAATGGAATATTTATTTCAGA |

Strain jl.27

| | | | |
|---|---|---|---|
| Identifier | 100 | 97.087 | CACTCGTGCCAAGAGCTAATGATAGCCCAAGCCCT |
| Sequence 1 | | | GTAACAATTACTTCAACTAATGATGGAGTTGTCGTA |
| (SEQ ID NO: 15) | | | ATTTGCGTAATTGCTTGAATAACGATAGGTGCTCCG |
| | | | AGTGAGATTAAACTCGATCCAGTACCTGGTACAGGT |
| | | | ACTCCATTCACTTGGATTGTAAGACCTCCTAATAGA |
| | | | CTTGCAGTTGCAGTGTTAGCGATAACAGTGATTTTA |
| | | | TAGAATCCAGTTTCACTAATTACGAAAGTATCAGCAT |
| | | | CTAATTGAGAAATTGCTGTACCAAACTGAGATCCAA |
| | | | CAGTATTAAATGGTACTGGATCATTAATTCCTAAATC |
| | | | TAAAGAAATCCCACCGGAGTTAAATGCGTATAGTCC |
| | | | TGCTGGAAGTCCTAGTCCTGATGGTCCAGTCGGTC |
| | | | CAGTCGGTCCAGTAGCCCCAGTCGGTCCAGTAGCT |
| | | | CCAGTAGCTCCAGTGTCACCAGTCGGCCCCGTAGG |
| | | | TCCAGTAGCTCCAGTGTCACCAGTCGGCCCCGTAG |
| | | | GTCCAGTAGCCCCAGTGTCACCAGTCGGCCCCGTA |
| | | | GGTCCAGTAGCCCCAGTGTCACCAGTCGGCCCCGT |
| | | | AGGTCCAGTAGTCCCAGTGTCACCAGTCGGTCCAG |
| | | | TCGGCCCCGTAGGTCCAGTCGGCCCAGTCGGCCC |
| | | | CGTAGGTCCAGTCGGTCCAGTTGGCCCGGTAGGTA |
| | | | CAGTCGGCCCCGTAGGTCCAGTCGGCCCAGTCGG |
| | | | CCCGGTCGGCCCAGTCGGAAGGGTAAACGGTGGT |
| | | | ATCGGTGGTAATGTAGGTCCTACAAGATTAGGGTCA |
| | | | AATGCACTAGCTGATAAAGATTCATCGGGATTTAAT |
| | | | CCATTTGAATAATTATTATTTGACATAAATTCACCTC |
| | | | CATAAAGCGTTCATTATATAGTAGATGCAAAACCGA |
| | | | AAGAAAATGACACGGACATTTGAATTATTGAAAAGA |
| | | | AATCTTAAACTACTTGAACAATTTAAAAAAATGGAAA |
| | | | GTTTAGTATATGTATAACATATGATTGATTTGGAAGA |
| | | | GGGTGATTATGTTGAACAAGCAAGGAATTACAATTA |
| | | | GTTTATGTATGATTGTTCGAGATGAGGAGGAGACAA |
| | | | TAGCCCGTTGTTTAGACACAGTTGAAAAAATTGTGG |
| | | | ATGAAATTATAGTGGTTGATACAGGCTCCGTCGATC |
| | | | GAACGAAAGAAATCGTAGAGAAATACACTTCTAACA |
| | | | TATATGATTTCCAGTGGATTGA |

### Example 13: Quorum Sensing and Metabolism Expression of S. aureus on Agar Plates when Mixed with Strain jl.68

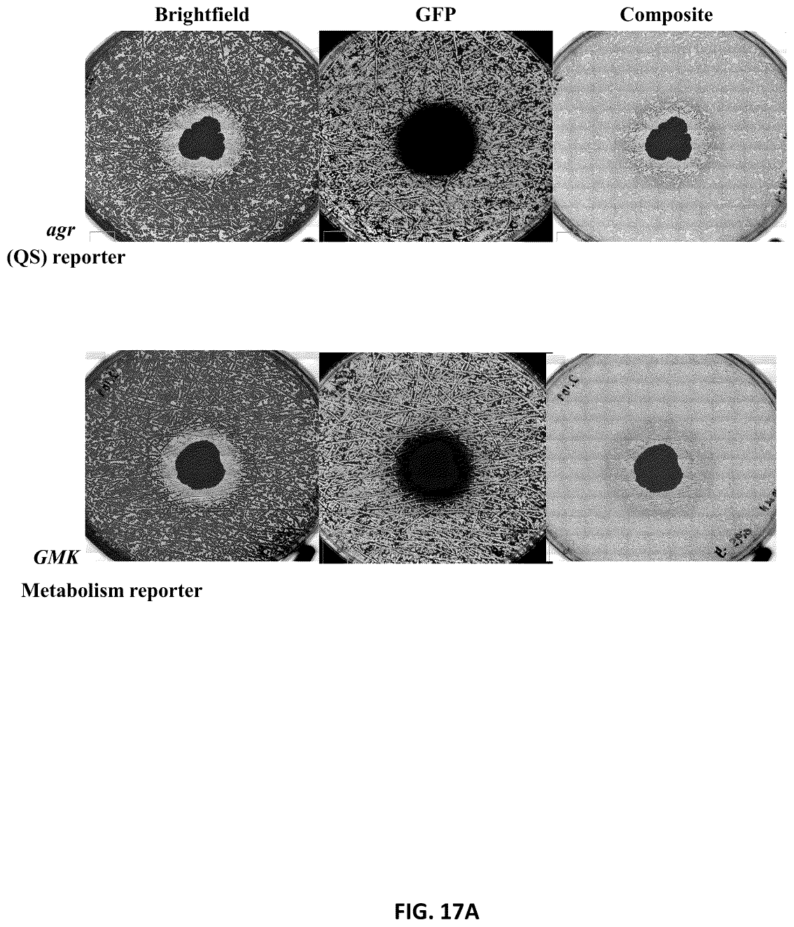

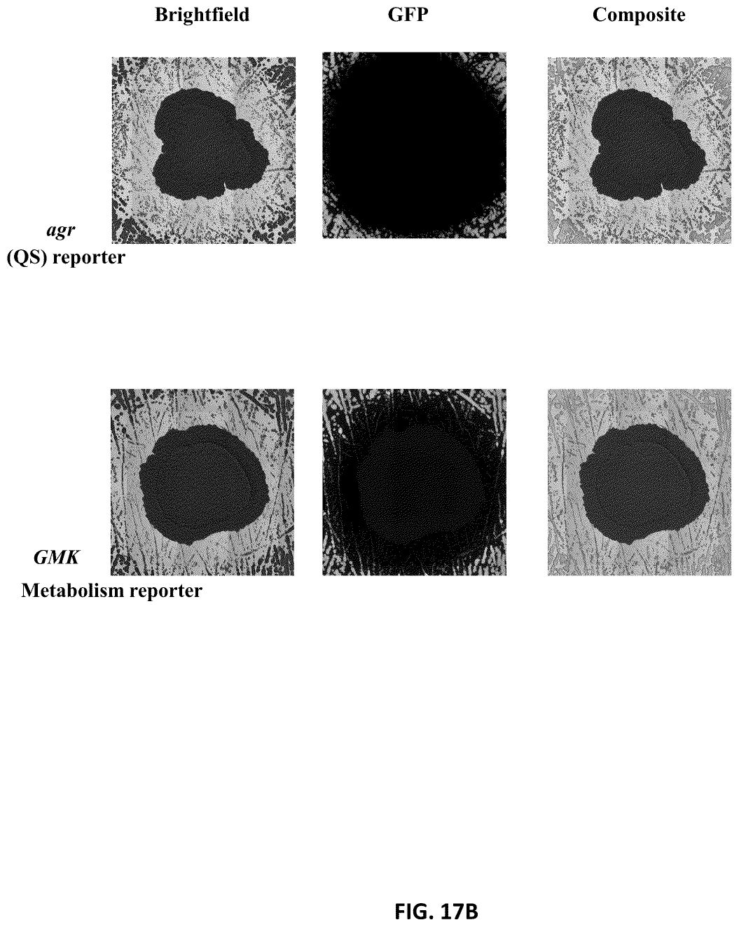

S. aureus reporter strains containing a GMK promoter fused GFP reporter or an agr promoter fused GFP reporter were spread on agar plates. 10 microliters of Strain jl.68 was plated in the center of the plate. Images of the plates were taken after incubation for 24 hours at 37° C. FIGS. 17A-B show the expression and growth of the S. aureus reporter strains. The expression of the reporters is shown in the GFP column. The growth of the S. aureus is shown in the brightfield column. The image suggests that cells of Strain jl.68 slow the growth of S. aureus and affect quorum sensing on agar plates.

### Example 14: Growth of S. aureus and Expression of S. aureus Genes after Mixture with Selected Strains and Supernatant of Selected Strains

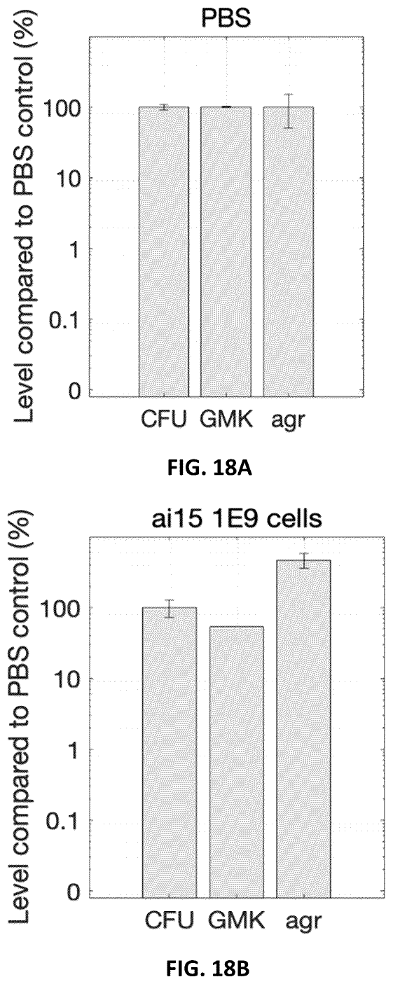

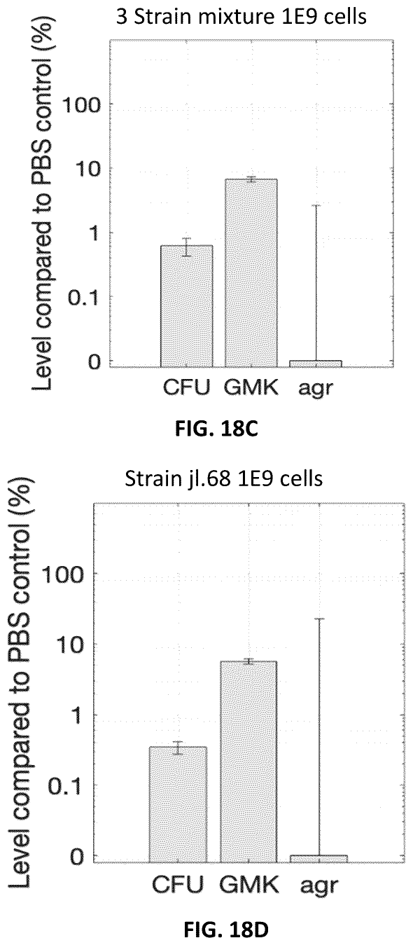

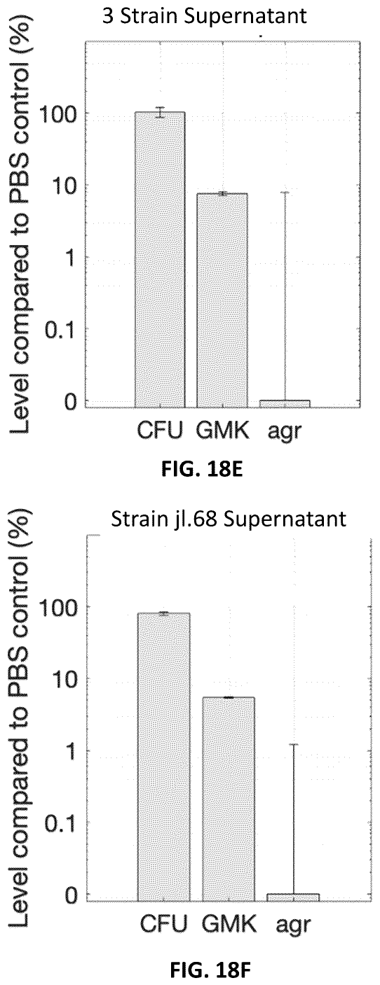

S. aureus reporter strains containing a GMK promoter fused GFP reporter or an agr promoter fused GFP reporter were grown in liquid cultures in the following conditions: grown in monoculture, mixed with S. aureus WT (control); mixed with Strain jl.27, Strain jl.68, and Strain jl.77; mixed with Strain jl.68 alone; mixed with supernatant from Strain jl.27, Strain jl.68, and Strain jl.77; or mixed with supernatant from strain jl.68 alone. The expression of GMK and agr were determined after incubation for 24 hours. The levels of expression and CFU were compared to a PBS control and calculated by (time 24−time 0)/(time 24 PBS−time 0 PBS). FIGS. 18A-F show the level compared to PBS on the Y-axis and the CFU, GMK expression and agr expression on the X-axis in the different growth conditions. The “agr” error bars appear large because less-than-zero values were set to 0. FIG. 18A shows the CFU of S. aureus and the expression of GMK and agr in a S. aureus monoculture control. FIG. 18B shows in a 1:1 coculture with ai.15 (S. aureus WT), there were increases in quorum sensing with little or no effect on cell viability or metabolism. FIGS. 18C-D show in a 1:1 coculture with Strain jl.27, Strain jl.68, and Strain jl.77 or with Strain jl.68 there was reduced cell viability (<1% (CFU)). Similarly, quorum sensing (agr-GFP) was reduced to negligible levels (near 0%) compared to the PBS control, and metabolism (GMK-GFP) was reduced to 5-10% as compared to the PBS control. FIGS. 18E-F show Strain jl.27, Strain jl.68, and Strain jl.77, or Strain jl.68 supernatant caused quorum sensing (agr-GFP) to be reduced (near 0%). Cell viability (CFU) was unaffected and metabolism (GMK-GFP) was reduced to 5-10% as compared to the PBS control. In liquid coculture with high-density S. aureus (10⁹ CFU/mL), the three strain combination of Strain jl.27, Strain jl.68, and Strain jl.77 and the single Strain jl.68 quenched quorum sensing (agr) and reduced viability (<1%). Similarly the supernatant maintained this effect on quorum sensing without reducing viability.

### Example 15: Metabolism and Quorum Sensing Expression of S. aureus after Mixture with Supernatant of a Selected Strain

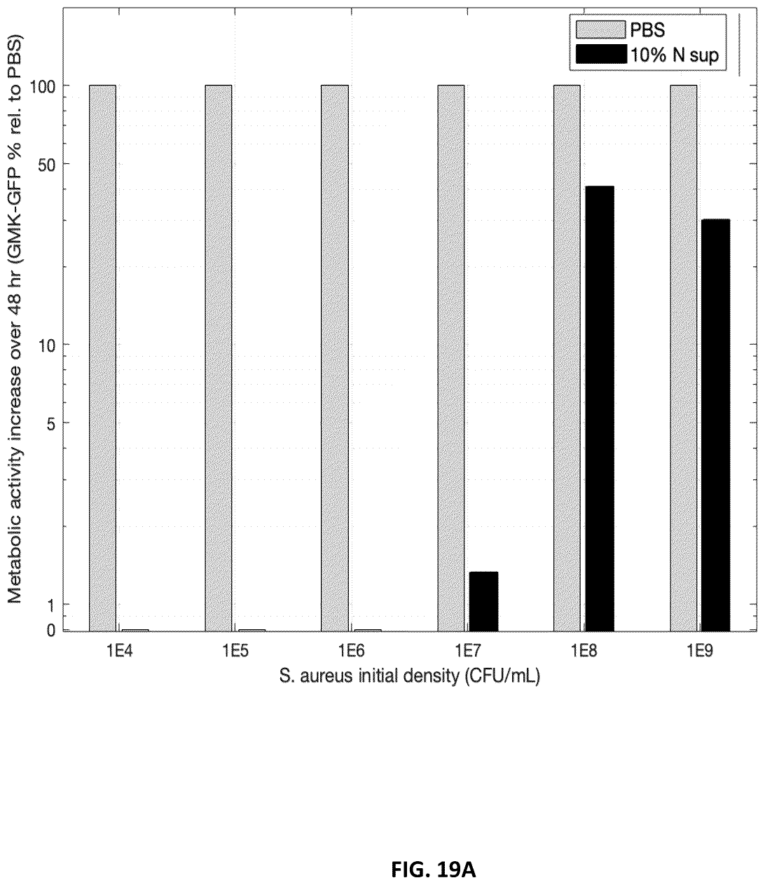

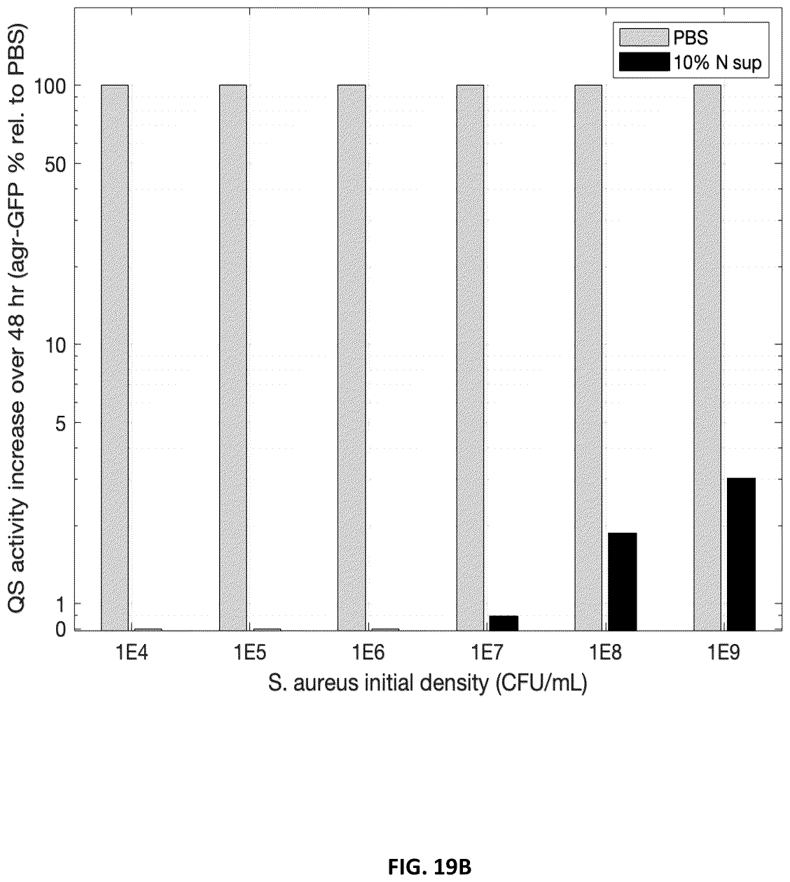

S. aureus reporter strains containing a GMK promoter fused GFP reporter or an agr promoter fused GFP reporter were grown in liquid cultures at increasing starting densities of: 1E4, 1E5, 1E6, 1E7, 1E8, or 1E9 CFU/ml. The liquid cultures were mixed with PBS or 10% (v/v) of supernatant from an overnight culture of Strain jl.68. The cultures were grown for 48 hours. The levels of expression were compared to a PBS control and calculated by (time 48−time 0)/(time 48 PBS−time 0 PBS). FIG. 19A shows a graph plotting the expression of GMK, and FIG. 19B shows the expression of agr. The graphs show the GMK or agr activity over 48 hours as compared to a PBS control on the Y-axis. The X-axis shows the different concentrations of S. aureus initial densities. Metabolism (and by extension, quorum sensing) appeared to be arrested for S. aureus low-density cultures (starting at or below 1E6 CFU/mL). While metabolism appeared to be partially restored for medium- and higher-density S. aureus cultures (20-30% relative to no-supernatant), quorum sensing increased only marginally (1-3% relative to no-supernatant). The results from this experiment may indicate supernatant slows metabolism, driving S. aureus into a more slowly growing regime in which it does not quorum sense and/or supernatant acts on quorum sensing, slowing overall metabolic activity.

### Example 16: Growth Inhibition of S. aureus after Mixture with Supernatant of a Selected Strain

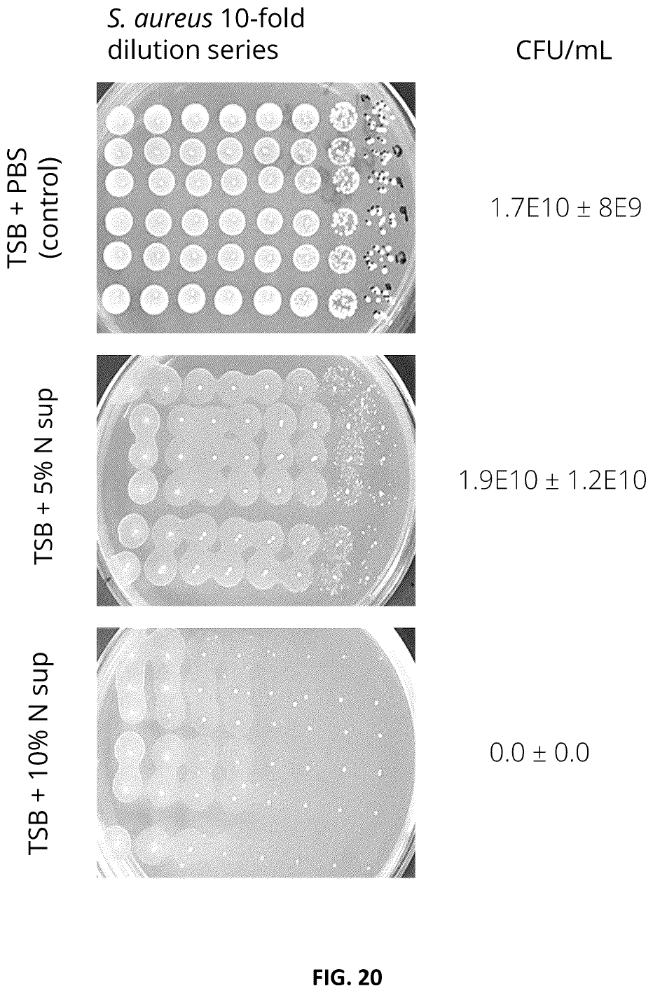

S. aureus containing an agr promoter fused GFP reporter was plated in 10-fold dilutions on TSB agar plates containing 0% (PBS control), 5% or 10% (v/v) supernatant from Strain jl.68. The plates were incubated for 18 hours at 37° C. and colonies were counted. FIG. 20 shows images of the plates containing the dilutions of S. aureus on the agar plates comprising the various percentages of supernatant from Strain jl.68. In the presence of unaltered TSB medium, individual colonies were countable around 6-7 10-fold dilutions of a saturated culture. They were bright and fluorescent, indicating quorum sensing was on. In the presence of 5% Strain jl.68 supernatant, colonies were visibly smaller and much less fluorescent, suggesting growth and quorum sensing were affected. Colony count did not change appreciably compared to the TSB-only control. At 18 hours, the colonies were hardly visible. In the presence of 10% Strain jl.68 supernatant, there were no countable colonies. A residue of the plated cultures was present and fluoresce was not detected. Occasionally, colonies emerged within the less-diluted cases that overcome the inhibitory effect of the supernatant. Together the data shows Strain jl.68 supernatant embedded into agar surface substantially eliminated S. aureus colony outgrowth and quorum sensing.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for reducing growth of Staphylococcus aureus on a skin of a subject in need thereof, comprising:
topically administering to the skin of the subject a composition, wherein the composition comprises:
bacterial strains that are purified and formulated in a form selected from a group consisting of an emulsion, a cream, a lotion, a gel, and a foam, wherein the bacterial strains comprise:
a first bacterial strain, which comprises a 16S rRNA sequence with at least 97% sequence identity to SEQ ID NO: 2;
a second bacterial strain, which comprises a 16S rRNA sequence with at least 97% sequence identity to SEQ ID NO: 4,
wherein the composition is in an amount sufficient for reduction of S. aureus on the skin of the subject in need thereof.

2. The method of claim 1, wherein the first bacterial strain comprises the 16S rRNA sequence with at least 97% sequence identity over at least 1000 bases to SEQ ID NO: 2.

3. The method of claim 1, wherein the second bacterial strain comprise the 16S rRNA sequence with at least 97% sequence identity over at least 1000 bases to SEQ ID NO: 4.

4. The method of claim 1, wherein the first bacterial strain further comprises a sequence with at least 97% sequence identity to SEQ ID NO: 7.

5. The method of claim 1, wherein the second bacterial strain further comprises a sequence with at least 97% sequence identity to SEQ ID NO: 11.

6. The method of claim 1, wherein the second bacterial strain further comprises a sequence with at least 97% sequence identity to SEQ ID NO: 13.

7. The method of claim 1, wherein the 16S rRNA sequence of the second bacterial strain comprises SEQ ID NO: 4 or SEQ ID NO: 5.

8. The method of claim 1, wherein the composition further comprises a third bacterial strain.

9. The method of claim 8, wherein the 16S rRNA sequence of the second bacterial strain comprises SEQ ID NO: 4, and the third bacterial strain comprises a 16s rRNA sequence that comprises SEQ ID NO: 5.

10. The method of claim 9, wherein the 16S rRNA sequence of the first bacterial strain comprises SEQ ID NO: 2, the 16S rRNA sequence of the second bacterial strain comprises SEQ ID NO: 4, and the 16S rRNA sequence of the third bacterial strain comprises SEQ ID NO: 5.

11. The method of claim 1, wherein the composition comprises at least 10\hat{ }3 colony forming units (CFU) of bacteria per gram of the composition.

12. The method of claim 1, wherein the composition comprises 10\hat{ }3 to 10\hat{ }12 colony forming units (CFU) of bacteria per gram of the composition.

13. The method of claim 1, wherein the bacterial strains are present in a ratio of CFUs of about 1:1 relative to each other.

14. The method of claim 1, wherein the composition further comprises an excipient.

15. The method of claim 1, wherein the composition further comprises thiamine or a salt thereof.

16. The method of claim 1, wherein the composition further comprises a sugar.

17. The method of claim 1, wherein the bacterial strains when contacted with S. aureus cause a reduction in expression of at least one of the following genes: gmk, agr, psma, saeR, ccpA, and SigB in S. aureus, wherein the reduction in expression is measured by a fluorescence reporter assay.

18. The method of claim 1, wherein the composition causes a reduction in expression of at least one of the following genes: gmk, agr, psma, saeR, ccpA, and SigB in S. aureus, as compared to the expression of the at least one of the following genes: gmk, agr, psma, saeR, ccpA, and SigB in S. aureus that is not contacted with the composition in the effective amount, wherein the reduction in expression is measured by a fluorescence reporter assay.

19. The method of claim 1, wherein the composition suppresses virulence of S. aureus as measured by a reduction in expression of at least one of the following genes: gmk, agr, psma, saeR, ccpA, and SigB in S. aureus, as compared to the expression of the at least one of the following genes: gmk, agr, psma, saeR, ccpA, and SigB in S. aureus that is not contacted with the composition in the effective amount wherein the reduction in expression is measured by a fluorescence reporter assay.

20. The method of claim 1, wherein the composition is located within a vial.

21. The method of claim 1, wherein the composition comprises the bacterial strains individually in an amount of at least 10\hat{ }4 to 10\hat{ }8 colony forming units (CFU) of bacteria per gram of the composition.
